(19) United States
(12) Patent Application Publication
(10) Pub. No.: US 2026/0078187 A1
(43) Pub. Date: Mar. 19, 2026

(54) METHODS OF TREATING CANCER WITH AN ANTI-PD-L1 ANTIBODY

(71) Applicant: Genentech, Inc., South San Francisco, CA (US)

(72) Inventors: Daniel ShinYu CHEN, Burlingame, CA (US); Cathleen AHEARN, South San Francisco, CA (US); Alan Bart SANDLER, South San Francisco, CA (US)

(21) Appl. No.: 19/319,392

(22) Filed: Sep. 4, 2025

Related U.S. Application Data
(62) Division of application No. 17/509,832, filed on Oct. 25, 2021, now abandoned.
Application No. 17/509,832 is a continuation of application No. PCT/US2020/030614, filed on Apr. 30, 2020.
(60) Provisional application No. 62/843,233, filed on May 3, 2019.

(51) Int. Cl. C07K 16/28; A61K 31/282; A61K 31/337; A61K 31/7048; A61K 39/00; A61K 39/395; A61P 35/00

(52) U.S. Cl. CPC C07K 16/2827; A61K 31/282; A61K 31/337; A61K 31/7048; A61K 39/3955; A61P 35/00; A61K 2039/505; A61K 2039/54; A61K 2039/545; C07K 2317/56

(57) ABSTRACT

The present disclosure relates to methods, uses, and kits related to treating cancers by administering an anti-PD-L1 antibody (e.g., atezolizumab) to a patient. In some embodiments, the anti-PD-L1 antibody is administered in 840 mg every 2 weeks or 1680 mg every 4 weeks for two or more cycles.

### CROSS-REFERENCE TO RELATED APPLICATIONS

This application is a divisional of U.S. application Ser. No. 17/509,832, filed on Oct. 25, 2021, which is a continuation of International Application No. PCT/US2020/030614, filed on Apr. 30, 2020, which claims priority to U.S. Provisional Application No. 62/843,233 filed on May 3, 2019, the contents of which are incorporated herein by reference in their entirety.

### SEQUENCE LISTING

The instant application contains a Sequence Listing which has been submitted electronically in XML format and is hereby incorporated by reference in its entirety. Said XML copy, created on Sep. 3, 2025, is named 50474-366004_Sequence_Listing_9_3_25 and is 22,306 bytes in size.

### TECHNICAL FIELD

The present disclosure relates to methods, uses, and kits related to treating cancers by administering an anti-PD-L1 antibody (e.g., atezolizumab).

### BACKGROUND

PDL1 is overexpressed in many cancers and is often associated with poor prognosis (Okazaki T et al., Intern. Immun. 2007 19(7):813) (Thompson RH et al., Cancer Res 2006, 66(7):3381). Interestingly, the majority of tumor infiltrating T lymphocytes predominantly express PD-1, in contrast to T lymphocytes in normal tissues and peripheral blood T lymphocytes indicating that up-regulation of PD-1 on tumor-reactive T cells can contribute to impaired antitumor immune responses (Blood 2009 114(8):1537). This may be due to exploitation of PDL1 signaling mediated by PDL1 expressing tumor cells interacting with PD-1 expressing T cells to result in attenuation of T cell activation and evasion of immune surveillance (Sharpe et al., Nat Rev 2002) (Keir ME et al., 2008 Annu. Rev. Immunol. 26:677). Therefore, inhibition of the PDL1/PD-1 interaction may enhance CD8+ T cell-mediated killing of tumors.

TECENTRIQ® (atezolizumab) is a humanized immunoglobulin G1 monoclonal antibody consisting of two heavy chains and two light chains. Atezolizumab targets human programmed death-ligand 1 (PD-L1) on tumor-infiltrating immune cells (ICs) and tumor cells, and inhibits its interaction with its receptors programmed death-1 (PD-1) and B7.1, both of which can provide inhibitory signals to T cells. Atezolizumab has been approved in over 71 countries as monotherapy for the treatment of 2L NSCLC, 2L metastatic UC, and/or 1L cisplatin-ineligible metastatic UC. For example, atezolizumab has been approved in the U.S. or Europe for the following indications: treatment of adult patients with locally advanced or metastatic urothelial carcinoma (UC) after prior platinum-containing chemotherapy, or who are considered cisplatin ineligible and whose tumors have a PD-L1 expression ≥5%, treatment of adult patients with locally advanced or metastatic non-small cell lung cancer (NSCLC) after prior chemotherapy; treatment of patients with locally advanced or metastatic UC who are not eligible for cisplatin-containing chemotherapy and whose tumors express PD-L1 (PD-L1 stained ICs covering 5% of the tumor area), or are not eligible for any platinum-containing chemotherapy regardless of level of tumor PD-L1 expression, or have disease progression during or after any platinum-containing chemotherapy or within 12 months of neoadjuvant or adjuvant chemotherapy; and treatment of patients with metastatic NSCLC who have disease progression during or after platinum-containing chemotherapy. Atezolizumab is also undergoing development as monotherapy and in combination with other targeted and cytotoxic agents for the treatment of patients with multiple solid and hematological tumors, including lung, renal, colorectal, and breast cancers.

All currently approved indications for atezolizumab are approved at a dose of 1200 mg as an intravenous (IV) infusion every 3 weeks (q3w) until disease progression or unacceptable toxicity occurs.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

Dosing schedules other than 1200 mg q3w would provide for greater flexibility for monotherapies and combination therapies that include atezolizumab. For example, an atezolizumab dosing schedule with administration every 4 weeks that provides a similar level of efficacy and safety as the approved q3w schedule would allow for greater patient convenience, particularly as part of a maintenance phase therapy.

In some aspects, provided herein are methods, kits, and uses for treating or delaying progression of cancer in a human patient, comprising administering to the human patient an anti-PD-L1 antibody in two or more 4-week or 28-day cycles at a dose of 1680 mg, wherein the anti-PD-L1 antibody is administered at a dose of 1680 mg per cycle in each of the two or more 4-week or 28-day cycles (e.g., the anti-PD-L1 antibody is administered once every 4 weeks or every 28 days to the human patient).

In some aspects, provided herein are methods, kits, and uses for treating or delaying progression of cancer in a human patient, comprising administering to the human patient an anti-PD-L1 antibody in two or more 2-week or 14-day cycles at a dose of 840 mg, wherein the anti-PD-L1 antibody is administered at a dose of 840 mg per cycle in each of the two or more 2-week or 14-day cycles (e.g., the anti-PD-L1 antibody is administered once every 2 weeks or every 14 days to the human patient).

In some aspects, the present disclosure provides methods for treating a human patient having cancer, comprising administering to the patient an anti-PD-L1 antibody at a dose of 840 mg every 2 weeks or 1680 mg every 4 weeks, wherein the anti-PD-L1 antibody comprises a heavy chain comprising HVR-H1 sequence of GFTFSDSWIH (SEQ ID NO:1), HVR-H2 sequence of AWISPYGGSTYYADSVKG (SEQ ID NO:2), and HVR-H3 sequence of RHWPGGFDY (SEQ ID NO:3), and a light chain comprising HVR-L1 sequence of RASQDVSTAVA (SEQ ID NO:4), HVR-L2 sequence of SASFLYS (SEQ ID NO:5), and HVR-L3 sequence of QQYLYHPAT (SEQ ID NO:6).

In some embodiments, the anti-PD-L1 antibody is administered on day 1 of each of the 2-week or 4-week cycles.

In some embodiments, the anti-PD-L1 antibody is administered to the patient in a maintenance phase of treatment. In some embodiments, the anti-PD-L1 antibody is administered to the patient in an induction phase of treatment.

In some embodiments, the methods described herein further comprise administering to the patient an additional therapeutic agent. In some embodiments, the additional therapeutic agent comprises a chemotherapeutic agent. In some embodiments, the chemotherapeutic agent is standard of care for the cancer. In some embodiments, the additional therapeutic agent comprises an antibody.

In some embodiments, the anti-PD-L1 antibody is administered to the patient by intravenous infusion. In some embodiments, the anti-PD-L1 antibody is administered to the patient by intravenous infusion over 60 minutes. In some embodiments, the anti-PD-L1 antibody is administered to the patient by intravenous infusion over 60 minutes in the initial infusion, and if the first infusion is tolerated, the anti-PD-L1 antibody is administered to the patient by intravenous infusion over 30 minutes in subsequence infusions. In some embodiments, the anti-PD-L1 antibody is administered to the patient by intravenous infusion over 30 minutes.

In some embodiments, the cancer is selected from the group consisting of breast cancer, colorectal cancer, lung cancer, renal cell carcinoma (RCC), ovarian cancer, melanoma, and bladder cancer. In some embodiments, the breast cancer is triple-negative breast cancer. In some embodiments, the lung cancer is non-small cell lung cancer or small cell lung cancer. In some embodiments, the bladder cancer is urothelial carcinoma. In some embodiments, the cancer is locally advanced or metastatic. In some embodiments, the cancer is locally advanced or metastatic urothelial carcinoma.

In some embodiments, the human patient has been treated with a platinum-containing chemotherapy prior to administration of the anti-PD-L1 antibody. In some embodiments, the human patient is ineligible for a platinum-containing chemotherapy. In some embodiments, the human patient has been treated with an adjuvant or neoadjuvant chemotherapy prior to administration of the anti-PD-L1 antibody.

In some embodiments, the cancer is locally advanced or metastatic non-small cell lung cancer, and wherein the patient has been treated with a chemotherapy prior to administration of the anti-PD-L1 antibody.

In some embodiments, the sample from the cancer of the patient comprises tumor-infiltrating immune cells that express PD-L1 and cover 1% or more of the tumor area, as assayed by immunohistochemistry (IHC).

In some embodiments of the methods described herein, the human patient is an adult human patient with locally advanced or metastatic urothelial carcinoma. In some embodiments of the methods described herein, the human patient is an adult human patient with locally advanced or metastatic urothelial carcinoma, wherein the anti-PD-L1 antibody is administered to the human patient after a prior platinum-containing chemotherapy. In some embodiments of the methods described herein, the human patient is an adult human patient with locally advanced or metastatic urothelial carcinoma, wherein the human patient is considered cisplatin ineligible, and whose tumours have a PD-L1 expression ≥5%.

In some embodiments of the methods described herein, the human patient has locally advanced or metastatic urothelial carcinoma, wherein the human patient is not eligible for cisplatin-containing chemotherapy and whose tumor(s) express PD-L1 (PD-L1 stained tumor-infiltrating immune cells [IC]covering ≥5% of the tumor area), as determined by a US FDA-approved test. In some embodiments of the methods described herein, the human patient has locally advanced or metastatic urothelial carcinoma, wherein the human patient is not eligible for any platinum-containing chemotherapy regardless of PD-L1 status. In some embodiments of the method described herein, the human patient has locally advanced or metastatic urothelial carcinoma, wherein the human patient has disease progression during or following any platinum-containing chemotherapy, or within 12 months of neoadjuvant or adjuvant chemotherapy.

In some embodiments of the methods described herein, the human patient has locally advance or metastatic urothelial carcinoma, wherein the human patient received a prior platinum-containing chemotherapy. In some embodiments of the methods described herein, the human patient has locally advance or metastatic urothelial carcinoma, wherein the human patient is considered cisplatin ineligible, and whose tumours have a PD-L1 expression ≥5%. In some embodiments, the human patient is an adult.

In some embodiments of the methods described herein, the human patient is an adult human patient with metastatic non-squamous non-small cell lung cancer (NSCLC), wherein the method comprises administration of an anti-PD-L1 antibody, bevacizumab, paclitaxel, and carboplatin, and wherein the method is a first-line treatment.

In some embodiments of the methods described herein, the human patient is an adult human patient with metastatic non-squamous non-small cell lung cancer (NSCLC), wherein the metastatic non-squamous NSCLC is an EGFR mutant or ALK-positive, wherein the method comprising administration of an anti-PD-L1 antibody, bevacizumab, paclitaxel, and carboplatin is indicated only after failure of appropriate targeted therapies, such as platinum-containing therapy, e.g., carboplatin, bevacizumab, vinflunine, docetaxel, or paclitaxel. In some embodiments, the metastatic non-squamous NSCLC is an EGFR mutant. In some embodiments, the metastatic non-squamous NSCLC is ALK-positive.

In some embodiments of the methods described herein, the human patient is an adult human patient with locally advanced or metastatic NSCLC after prior chemotherapy, wherein the method comprising administration of an anti-PD-L1 antibody is indicated for monotherapy.

In some embodiments of the methods described herein, the human patient is an adult human patient with locally advanced or metastatic NSCLC after prior chemotherapy, wherein the metastatic non-squamous NSCLC is an EGFR mutant or ALK-positive, wherein the human patient received targeted therapies, such as platinum-containing therapy, e.g., carboplatin, bevacizumab, vinflunine, docetaxel, or paclitaxel, before performing a method described herein.

In some embodiments of the methods described herein, the human patient has metastatic non-squamous non-small cell lung cancer (NSCLC) with no EGFR or ALK genomic tumor aberrations. In some embodiments of the methods described herein, the human patient has metastatic non-squamous non-small cell lung cancer (NSCLC) with no EGFR or ALK genomic, wherein the method comprises wherein the method comprises administration of an anti-PD-L1 antibody, bevacizumab, paclitaxel, and carboplatin, and wherein the method is a first-line treatment.

In some embodiments of the methods described herein, the human patient has metastatic NSCLC, wherein the human patient progressed during or following platinum-containing chemotherapy, wherein the indication is an anti-PD-L1 antibody as a single-agent.

In some embodiments of the methods described herein, the human patient has metastatic NSCLC having an EGFR or ALK genomic tumor aberration, wherein the human patient failed a targeted therapy for a non-small cell lung cancer, wherein the method comprises administering to the human patient an anti-PD-L1 antibody in combination with bevacizumab, paclitaxel, and carboplatin.

In some embodiments of the methods described herein, the human patient has metastatic non-small cell lung cancer, and wherein the human patient progressed during or following platinum-containing chemotherapy. In some embodiments, the method comprises administering to the human patient an anti-PD-L1 antibody as a single agent. In some embodiments, wherein the human patient has an EGFR or ALK genomic tumor aberrations, the patient has progressed on a targeted therapy. In some embodiments, wherein the human patient has an EGFR or ALK genomic tumor aberrations, the patient has progressed on an FDA-approved therapy.

In some embodiments of the methods described herein, the human patient has locally advanced or metastatic non-small cell lung cancer, wherein the human patient has received prior chemotherapy.

In some embodiments of the methods described herein, the human patient has locally advanced or metastatic triple-negative breast cancer. In some embodiments of the methods described herein, the human patient has locally advanced or metastatic triple-negative breast cancer that is unresectable locally advanced or metastatic triple-negative breast cancer. In some embodiments of the methods described herein, the human patient has a tumour that expresses PD-L1 (PD-L1 stained tumor-infiltrating immune cells [IC] of any intensity covering ≥1% of the tumor area), as determined by an FDA-approved test.

In another aspect, the present disclosure provides methods for treating a human patient having locally advanced or metastatic urothelial carcinoma, comprising administering to the patient an anti-PDL1 antibody at a dose of 840 mg every 2 weeks or 1680 mg every 4 weeks, wherein the anti-PD-L1 antibody comprises a heavy chain comprising HVR-H1 sequence of GFTFSDSWIH (SEQ ID NO:1), HVR-H2 sequence of AWISPYGGSTYYADSVKG (SEQ ID NO:2), and HVR-H3 sequence of RHWPGGFDY (SEQ ID NO:3), and a light chain comprising HVR-L1 sequence of RASQDVSTAVA (SEQ ID NO:4), HVR-L2 sequence of SASFLYS (SEQ ID NO:5), and HVR-L3 sequence of QQYLYHPAT (SEQ ID NO:6). In some embodiments, the patient (i) is not eligible for cisplatin-containing chemotherapy and whose tumors express PD-L1 (PD-L1 stained tumor-infiltrating immune cells [IC]covering ≥5% of the tumor area), (ii) is not eligible for any platinum-containing chemotherapy regardless of PD-L1 status, or (iii) has disease progression during or following any platinum-containing chemotherapy, or within 12 months of neoadjuvant or adjuvant chemotherapy.

In another aspect, the present disclosure provides methods for treating a human patient having non-small cell lung cancer (NSCLC), comprising administering to the patient an anti-PDL1 antibody as a single agent at a dose of 840 mg every 2 weeks or 1680 mg every 4 weeks, wherein the anti-PD-L1 antibody comprises a heavy chain comprising HVR-H1 sequence of GFTFSDSWIH (SEQ ID NO:1), HVR-H2 sequence of AWISPYGGSTYYADSVKG (SEQ ID NO:2), and HVR-H3 sequence of RHWPGGFDY (SEQ ID NO:3), and a light chain comprising HVR-L1 sequence of RASQDVSTAVA (SEQ ID NO:4), HVR-L2 sequence of SASFLYS (SEQ ID NO:5), and HVR-L3 sequence of QQYLYHPAT (SEQ ID NO:6). In some embodiments, the patient has (i) metastatic NSCLC and disease progression during or following platinum-containing chemotherapy, or (ii) has EGFR or ALK genomic tumor aberrations.

In another aspect, the present disclosure provides methods for treating a human patient having non-small cell lung cancer (NSCLC), comprising (a) administering to the patient an anti-PDL1 antibody at a dose of 1200 mg every 3 weeks in combination with bevacizumab, paclitaxel and carboplatin for 4-6 cycles of paclitaxel and carboplatin; and (b) if bevacizumab is discontinued, administering to the patient an anti-PDL1 antibody at a dose of 840 mg every 2 weeks or 1680 mg every 4 weeks; wherein the anti-PD-L1 antibody comprises a heavy chain comprising HVR-H1 sequence of GFTFSDSWIH (SEQ ID NO:1), HVR-H2 sequence of AWISPYGGSTYYADSVKG (SEQ ID NO:2), and HVR-H3 sequence of RHWPGGFDY (SEQ ID NO:3), and a light chain comprising HVR-L1 sequence of RASQDVSTAVA (SEQ ID NO:4), HVR-L2 sequence of SASFLYS (SEQ ID NO:5), and HVR-L3 sequence of QQYLYHPAT (SEQ ID NO:6). In some embodiments, the patient has metastatic non-squamous NSCLC with no EGFR or ALK genomic tumor aberrations. In some embodiments, the method is for first-line treatment for metastatic non-squamous NSCLC with no EGFR or ALK genomic tumor aberrations. In some embodiments, bevacizumab is administered at 15 mg/kg, paclitaxel is administered at 175 mg/m² or 200 mg/m², and carboplatin is administered at AUC 6 mg/mL/min, wherein the

In another aspect, the present disclosure provides methods for treating a human patient having small cell lung cancer (SCLC), comprising (a) administering to the patient an anti-PDL1 antibody at a dose of 1200 mg every 3 weeks in combination with carboplatin and etoposide for 4 cycles of carboplatin and etoposide; and (b) following completion of (a), administering to the patient an anti-PDL1 antibody at a dose of 840 mg every 2 weeks or 1680 mg every 4 weeks; wherein the anti-PD-L1 antibody comprises a heavy chain comprising HVR-H1 sequence of GFTFSDSWIH (SEQ ID NO:1), HVR-H2 sequence of AWISPYGGSTYYADSVKG (SEQ ID NO:2), and HVR-H3 sequence of RHWPGGFDY (SEQ ID NO:3), and a light chain comprising HVR-L1 sequence of RASQDVSTAVA (SEQ ID NO:4), HVR-L2 sequence of SASFLYS (SEQ ID NO:5), and HVR-L3 sequence of QQYLYHPAT (SEQ ID NO:6). In some embodiments, the patient has extensive-stage small cell lung cancer (ES-SCLC). In some embodiments, carboplatin is administered at AUC 5 mg/mL/min on day 1, and etoposide is administered at 100 mg/m² intravenously on day 1, 2, and 3 of each 21-day cycle. In some embodiments, the treatment is for the first-line treatment.

In another aspect, the present disclosure provides methods for treating a human patient having unresectable locally advanced or metastatic TNBC, comprising administering to the human patient an anti-PD-L1 antibody at a dose of 840 mg every 2 weeks, wherein the method further comprises administering to the human patient paclitaxel at a dose of 100 mg/m² on days every week, wherein the anti-PD-L1 antibody comprises a heavy chain comprising HVR-H1 sequence of GFTFSDSWIH (SEQ ID NO:1), HVR-H2 sequence of AWISPYGGSTYYADSVKG (SEQ ID NO:2), and HVR-H3 sequence of RHWPGGFDY (SEQ ID NO:3), and a light chain comprising HVR-L1 sequence of RASQDVSTAVA (SEQ ID NO:4), HVR-L2 sequence of SASFLYS (SEQ ID NO:5), and HVR-L3 sequence of QQYLYHPAT (SEQ ID NO:6). In some embodiments, the method comprises administering to the human patient an anti-PD-L1 antibody at a dose of 840 mg on days 1 and 15 of a 28-day cycle and administering to the human patient paclitaxel protein-bound on days 1, 8, and 15 of a 28-day cycle. In some embodiments, the human patient has a tumor that expresses PD-L1 (PD-L1 stained tumor-infiltrating immune cells [IC]covering ≥1% of the tumor area).

In some embodiments of the methods described herein, the cancer is breast cancer (e.g., unresectable locally advanced or metastatic TNBC), and the methods further comprise administering a taxane (e.g., paclitaxel or protein-bound paclitaxel) in combination with the anti-PD-L1 antibody (e.g., atezolizumab).

In some embodiments of the methods described herein, the anti-PD-L1 antibody is administered to the patient by intravenous infusion. In some embodiments of the methods described herein, the anti-PD-L1 antibody is administered to the patient by intravenous infusion over 60 minutes. In some embodiments of the methods described herein, the anti-PD-L1 antibody is administered to the patient by intravenous infusion over 60 minutes in the initial infusion, and if the first infusion is tolerated, the anti-PD-L1 antibody is administered to the patient by intravenous infusion over 30 minutes in subsequence infusions. In some embodiments of the methods described herein, the anti-PD-L1 antibody is administered to the patient by intravenous infusion over 30 minutes.

In some embodiments of the methods described herein, the patient is an adult patient.

In some embodiments of the methods described herein, the anti-PD-L1 antibody comprises a heavy chain comprising HVR-H1 sequence of GFTFSDSWIH (SEQ ID NO:1), HVR-H2 sequence of AWISPYGGSTYYADSVKG (SEQ ID NO:2), and HVR-H3 sequence of RHWPGGFDY (SEQ ID NO:3), and a light chain comprising HVR-L1 sequence of RASQDVSTAVA (SEQ ID NO:4), HVR-L2 sequence of SASFLYS (SEQ ID NO:5), and HVR-L3 sequence of QQYLYHPAT (SEQ ID NO:6).

In some embodiments of the methods described herein, the heavy chain of the anti-PD-L1 antibody comprises a heavy chain variable (VH) domain comprising the sequence of EVQLVESGGGLVQPGGSLRLSCAASGFTFSDSWIHWVRQAPGKGLEWVAWISPYGGST YYADSVKGRFTISADTSKNTAYLQMNSLRAEDTAVYYCARRHWPGGFDYWGQGTLVT VSS (SEQ ID NO:7), and wherein the light chain of the anti-PD-L1 antibody comprises a light chain variable (VL) domain comprising the sequence of

(SEQ ID NO: 8)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In some embodiments of the methods described herein, the anti-PD-L1 antibody is atezolizumab.

In another aspect, the present disclosure provides kits, the kits comprising a unit dose of an anti-PD-L1 antibody in a pharmaceutically acceptable carrier for use in any one of the methods described herein. In some embodiments, the unit dose of the anti-PD-L1 antibody is 840 mg. In some embodiments, the unit dose of the anti-PD-L1 antibody is provided in 14 mL of a solution comprising the pharmaceutically acceptable carrier

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE FIGURES

FIG. 1 shows the statistically significant parameter-covariate relationships identified for the popPK model for atezolizumab. BWT=body weight (kg); i denotes a specific patient; ALBU=albumin (g/L); tumor burden (mm); ATAG=post-baseline status of anti-therapeutic antibodies.

FIG. 2 provides sensitivity plots comparing the effect of covariates (BW, albumin, tumor burden, gender, Atag) on atezolizumab steady-state exposure parameters AUCₛₛ (left), C_{max,ss} (middle), and C_{min,ss} (right). No covariate effect induced more than 30% change in exposure from the typical patient except for BW. Atag=post-baseline status of anti-therapeutic antibodies; AUCₛₛ=area under the serum concentration time curve at steady-state; C_{max,ss}=maximum observed serum concentration at steady-state; C_{min,ss}=minimum observed serum concentration at steady-state. Final model estimate, as represented by the black vertical line and value, refers to the predicted steady-state exposure of atezolizumab 1200 mg q3w in a typical patient (male) with covariates equal to medians. Grey areas, dark and light represent 20% and 30% of change from the base, respectively. The top bar shows the 10^{th} and 90^{th} percentile ([p10-p90]) exposure range across the population receiving 1200 mg q3w. Each horizontal bar represents the influence of a single covariate on the exposure metric. The label at left end of the bar represents the covariate being evaluated with values of the 10^{th} and 90^{th} percentiles ([p10-p90]) of the covariate distribution. The length of each bar describes the potential impact of that particular covariate on atezolizumab exposure, with the percent change of exposure from the base (blue values).

FIGS. 3A-3B provide prediction-corrected visual predictive checks (pcVPCs) using the phase I population pharmacokinetics (popPK) model of atezolizumab data from the IMvigor210 (FIG. 3A) and IMvigor211 (FIG. 3B) clinical trials. The pcVPC's suggested that the Phase I popPK Model was adequate to predict atezolizumab PK data in all patients from IMvigor210 and IMvigor211. CI=confidence interval.

FIGS. 4A-4B provide prediction-corrected visual predictive checks (pcVPCs) using the phase I popPK model of pooled atezolizumab data from the BIRCH, FIR, and POPLAR (FIG. 4A), as well as OAK (FIG. 4B), clinical trials. The pcVPCs by study suggested that the Phase I popPK Model was adequate to predict atezolizumab PK data in BIRCH (all Cohorts) as well as in FIR (all Cohorts) and OAK. A trend to negative population-level predictions and residuals was observed for POPLAR, but this trend was resolved in individual predictions and residuals, indicating that the Phase I popPK model allowed reliable and robust Bayesian estimation of individual parameter in all studies. CI=confidence interval.

FIGS. 5A-5C provide the logistic regression of objective response rate versus atezolizumab exposure metrics cycle 1 AUC (FIG. 5A), cycle 1 Cₘᵢₙ (FIG. 5B), and AUCₛₛ(FIG. 5C) for patients with 1L cisplatin-ineligible urothelial carcinoma in IMvigor210 receiving atezolizumab 1200 mg q3w. There was no statistically significant ER relationship between probability of response and atezolizumab exposure with any of the exposure metrics considered. 1L=first-line; AUC=area under the curve; Cₘᵢₙ=minimum concentration of the cycle; AUCₛₛ=area under the curve at steady-state; CI=confidence interval; CR=complete response; N=number of patients; p=p value of Wald test in logistic regression of proportion of responders versus exposure; PR=partial response; q3w=every three weeks. The grey solid line and shaded area represent the logistic regression slope model and 95% prediction interval. The filled circles and error bar represent the proportion of responders in exposure quartiles and 95% CI. The vertical lines are the limits of the exposure quartiles. The crosses are the patient response events (0: No, 1: Yes). The triangle and two-headed arrow represent the mean exposure and exposure interval between the 10th and the 90^{th} percentile for patients receiving 1200 mg atezolizumab, respectively.

FIGS. 6A-6C provide the logistic regression of objective response rate versus atezolizumab exposure metrics cycle 1 AUC (FIG. 6A), cycle 1 Cₘᵢₙ (FIG. 6B), and AUCₛₛ (FIG. 6C) for patients with 2L urothelial carcinoma in IMvigor210 receiving atezolizumab 1200 mg q3w. There was no statistically significant ER relationship between probability of response and atezolizumab exposure with any of the exposure metrics considered. 2L=second-line; AUC=area under the curve; Cₘᵢₙ=minimum concentration of the cycle; AUCₛₛ=area under the curve at steady-state; CI=confidence interval; CR=complete response; N=number of patients; p=p value of Wald test in logistic regression of proportion of responders versus exposure; PR=partial response; q3w=every three weeks. The grey solid line and shaded area represent the logistic regression slope model and 95% prediction interval. The filled circles and error bar represent the proportion of responders in exposure quartiles and 95% CI. The vertical lines are the limits of the exposure quartiles. The crosses are the patient response events (0: No, 1: Yes). The triangle and two-headed arrow represent the mean exposure and exposure interval between the 10th and the 90^{th} percentile for patients receiving 1200 mg atezolizumab, respectively.

FIG. 7 provides the logistic regression of objective response rate versus atezolizumab exposure metric cycle 1 AUC for patients with 2L urothelial carcinoma in IMvigor211 receiving 1200 mg atezolizumab. No statistically significant ER relationships (cycle 1 AUC) were identified with ORR following atezolizumab 1200 mg q3w. 2L=second-line; AUC=area under the curve; CI=confidence interval; CR=complete response; N=number of patients; p=p value of Wald test in logistic regression of proportion of responders versus exposure; PR=partial response; q3w=every three weeks. The grey solid line and shaded area represent the logistic regression slope model and 95% prediction interval. The filled circles and error bar represent the proportion of responders in exposure quartiles and 95% CI. The vertical lines are the limits of the exposure quartiles. The crosses are the patient response events (0: No, 1: Yes). The triangle and two-headed arrow represent the mean exposure and exposure interval between the 10th and the 90^{th} percentile for patients receiving 1200 mg atezolizumab, respectively.

FIGS. 8A-8D provide the logistic regression of objective response rate versus atezolizumab exposure metrics cycle 1 Cₘᵢₙ (FIG. 8A), cycle 1 AUC (FIG. 8B), AUCₛₛ (FIG. 8C), and versus patient body weight (FIG. 8D) for patients with NSCLC in BIRCH receiving 1200 mg atezolizumab q3w. For BIRCH, of the exposure metrics associated with a trend toward increased probability of response with atezolizumab exposure, the p-value associated with AUCₛₛ was the lowest (p=0.0005343). AUC=area under the curve; Cₘᵢₙ=minimum concentration of the cycle; AUCₛₛ=area under the curve at steady-state; CI=confidence interval; Cₘᵢₙ=minimum concentration of the cycle; CR=complete response; IC=immune cell; PR=partial response; N=number of patients; p=p value of Wald test in logistic regression of proportion of responders versus exposure; q3w=every 3 weeks. The grey solid line and shaded area represent the logistic regression slope model and 95% prediction interval. The filled circles and error bar represent the proportion of responders in exposure quartiles and 95% CI. The vertical lines are the limits of the exposure quartiles. The crosses are the patient response events (0: No, 1: Yes). The triangle and two-headed arrow represent the mean exposure and exposure interval between the 10^{th} and the 90^{th} percentile for patients receiving 1200 mg atezolizumab, respectively.

FIGS. 9A-9D provide the logistic regression of objective response rate versus atezolizumab exposure metrics cycle 1 Cₘᵢₙ (FIG. 9A), cycle 1 AUC (FIG. 9B), AUCₛₛ (FIG. 9C), and versus patient body weight (FIG. 9D) for patients with NSCLC in OAK receiving 1200 mg atezolizumab q3w. For OAK, of the exposure metrics associated with a trend toward increased probability of response with atezolizumab exposure, the p-value associated with AUCₛₛ was the lowest. AUC=area under the curve; Cₘᵢₙ=minimum concentration of the cycle; AUCₛₛ=area under the curve at steady-state; CI=confidence interval; Cₘᵢₙ=minimum concentration of the cycle; CR=complete response; IC=immune cell; PR=partial response; N=number of patients; p=p-value of Wald test in logistic regression of proportion of responders versus exposure; q3w=every 3 weeks. The grey solid line and shaded area represent the logistic regression slope model and 95% prediction interval. The filled circles and error bar represent the proportion of responders in exposure quartiles and 95% CI. The vertical lines are the limits of the exposure quartiles. The crosses are the patient response events (0: No, 1: Yes). The triangle and two-headed arrow represent the mean exposure and exposure interval between the 10^{th} and the 90^{th} percentile for patients receiving 1200 mg atezolizumab, respectively.

FIGS. 10A-10C provide the logistic regression of objective response rate versus atezolizumab exposure metrics cycle 1 Cₘᵢₙ (FIG. 10A), cycle 1 AUC (FIG. 10B), and AUCₛₛ(FIG. 10C) for patients with NSCLC in POPLAR receiving atezolizumab 1200 mg q3w. There was no statistically significant ER relationship between probability of response and atezolizumab exposure with any of the exposure metrics considered. AUC=area under the curve; Cₘᵢₙ=minimum concentration of the cycle; AUCₛₛ=area under the curve at steady-state; CI=confidence interval; CR=complete response; N=number of patients; p=p value of Wald test in logistic regression of proportion of responders versus exposure; PR=partial response; q3w=every three weeks. The grey solid line and shaded area represent the logistic regression slope model and 95% prediction interval. The filled circles and error bar represent the proportion of responders in exposure quartiles and 95% CI. The vertical lines are the limits of the exposure quartiles. The crosses are the patient response events (0: No, 1: Yes). The triangle and two-headed arrow represent the mean exposure and exposure interval between the 10th and the 90^{th} percentile for patients receiving 1200 mg atezolizumab, respectively.

FIGS. 11A-11B provide a simulation of the overall survival (OS) model after correcting for the imbalance of prognostic factors. The simulation of the OS model for NSCLC patients in POPLAR (FIG. 11A) after correcting for the imbalance of prognostic factors (number of metastatic sites and albumin level) across AUCₛₛ tertiles and docetaxel groups suggested that all patients would benefit from atezolizumab treatment. The simulation of the OS model for NSCLC patients in OAK (FIG. 11B) after correction of the imbalance of prognostic factors (baseline BSLD, albumin, ECOG performance status, and LDH level) across AUCₛₛ tertiles and docetaxel groups suggested that all patients would benefit from treatment with atezolizumab. AUCₛₛ=median and range of area under the curve at steady-state in μg·day/mL; HR=hazard ratio, CI=confidence interval; NSCLC=non-small cell lung cancer; q3w=every 3 weeks.

FIG. 12 provides Kaplan-Meier plots of OS by quartiles of BW for patients with NSCLC in OAK receiving 1200 mg atezolizumab q3w. The Kaplan-Meier plots suggest that heavier weight patients have similar OS to lighter weight patients. N=number of patients; NSCLC=non-small cell lung cancer; OS=overall survival; Q1=first quartile; Q2=second quartile; Q3=third quartile; Q4=fourth quartile; q3w=every 3 weeks; for the interval notations, a is included and b is excluded. The plain and dotted lines are Kaplan-Meier estimations. The crosses are censored observed values.

FIGS. 13A-13B provide the logistic regression of the proportion of response (CR+PR) versus atezolizumab exposure metrics cycle 1 AUC (FIG. 13A) and cycle 1 Cₘᵢₙ (FIG. 13B) for pooled patients with locally advanced or metastatic NSCLC or UC. For FIG. 13A, for legibility, 1 extreme AUC value (>15,000 μg·day/mL) is not displayed on the plot. Wald P values from logistic regression of proportion of responders vs. exposure are displayed. Grey solid lines and shaded areas represent the logistic regression slope model and 95% PI. Filled circles and error bars represent the proportions of responders in exposure quartiles and 95% CI; vertical lines are the limits of the exposure quartiles. Cross markings (x) represent response events (0: no, 1: yes). Triangle and 2-headed arrows represent the mean exposure and exposure interval between the 10th and 90th percentiles, respectively, for patients receiving atezolizumab 1200 mg. Cycle 1 AUC corresponds to the AUC during the first 3 weeks after treatment start and with PK parameters estimated based on cycle 1 data only. AUC=area under the concentration-time curve; Cₘᵢₙ=minimum (trough) serum atezolizumab concentration; CR=complete response; N=number of patients; NSCLC=non-small cell lung cancer; PI=prediction interval; PK=pharmacokinetics; PR=partial response; UC=urothelial carcinoma.

FIGS. 14A-14B provide validation of the TGI-OS model in simulating OS distributions by AUC (cycle 1, μg·day/mL) quartiles. Observed Kaplan-Meier OS distributions with censored data (+symbol) from OAK (NSCLC) (FIG. 14A) and IMvigor211 (UC) (FIG. 14B) are plotted. Shaded areas represent 95% PIs for OS distributions. For interval notation format [a, b), a is included and b is excluded, such that a≤x<b. AUC area under the concentration-time curve (0 to 21 days), NSCLC=non-small cell lung cancer; OS=overall survival; PI=prediction interval; TGI=tumor growth inhibition; UC=urothelial carcinoma.

FIGS. 15A-15B provide validation of the TGI-OS model in simulating HRs (atezolizumab vs comparator) by cycle 1 AUC quartiles for patients with original covariates. Forest plots for OS HRs from OAK (NSCLC) (FIG. 15A) and IMvigor211 (UC) (FIG. 15B) are shown. Observed HRs are shown as squares, and model-predicted HRs are shown as diamonds, with bars indicating 95% PIs (1000 replicates). Atezo=atezolizumab; AUC=area under the concentration-time curve; Chemo=chemotherapy; Cₘᵢₙ=minimum (trough) serum atezolizumab concentration; Doce=docetaxel; HR=hazard ratio; NSCLC=non-small cell lung cancer; OS=overall survival; PI=prediction interval; TGI=tumor growth inhibition; UC=urothelial carcinoma.

FIGS. 16A-16B provide predicted OS HRs (atezolizumab vs comparator) by cycle 1 AUC quartiles for patients with median covariates. Forest plots for OS HRs from OAK (NSCLC) (FIG. 16A) and IMvigor211 (UC) (FIG. 16B) are shown. Model-predicted HRs are shown as diamonds, with bars indicating 95% PIs (1000 replicates). Atezo=atezolizumab; AUC=area under the concentration-time curve; Chemo=chemotherapy; Doce=docetaxel; HR=hazard ratio; NSCLC=non-small cell lung cancer; OS=overall survival; PI=prediction interval; UC=urothelial carcinoma.

FIGS. 17A-17C provide the logistic regression of the proportion of patients experiencing AE of Grade ≥3 versus atezolizumab exposure metrics cycle 1 AUC (FIG. 17A), cycle 1 Cₘₐₓ (FIG. 17B), and AUCₛₛ (FIG. 17C) for patients in studies PCD4989g (Urothelial Carcinoma Cohort) and IMvigor210 (Cohorts 1 and 2) for atezolizumab Doses 15 mg/kg and 1200 mg q3w. The analysis of the incidence of AEG35 (AE of Grade≥3) did not show any statistically significant ER relationship with any exposure metric investigated. AUC=area under the concentration-time curve; Cₘₐₓ=maximum concentration in serum; AUCₛₛ=AUC at steady state; AE=adverse events; CI=confidence interval; N=number of patients; p=p value of Wald test in logistic regression of incidence versus exposure; q3w=every three weeks. The thick solid line and shaded area represent the logistic regression slope model and 95% prediction interval. The filled circles and error bar represent the incidence in exposure quartiles and 95% CI. The vertical lines are the limits of the exposure quartiles. The cross is AE (0: No, 1: Yes). The triangle and two-headed arrow represent the mean exposure and exposure interval between the 10^{th} and the 90^{th} percentile for patients receiving 1200 mg atezolizumab, respectively.

FIGS. 18A-18B provide the logistic regression of the proportion of patients experiencing AE of Grade 3 versus atezolizumab exposure metrics cycle 1 AUC (FIG. 18A), and cycle 1 Cₘₐₓ (FIG. 18B) for patients in study IMvigor211 receiving atezolizumab 1200 mg q3w. The analysis of the incidence of AEG35 did not show any statistically significant ER relationship with any exposure metric investigated. AUC=area under the concentration-time curve; Cₘₐₓ=maximum concentration in serum; AE=adverse events; CI=confidence interval; N=number of patients; p=p value of Wald test in logistic regression of incidence versus exposure; q3w=every three weeks. The thick solid line and shaded area represent the logistic regression slope model and 95% prediction interval. The filled circles and error bar represent the incidence in exposure quartiles and 95% CI. The vertical lines are the limits of the exposure quartiles. The cross is AE (0: No, 1: Yes). The triangle and two-headed arrow represent the mean exposure and exposure interval between the 10^{th} and the 90^{th} percentile for patients receiving 1200 mg atezolizumab, respectively.

FIGS. 19A-19C provide the logistic regression of the proportion of patients experiencing AESI versus atezolizumab exposure metrics cycle 1 AUC (FIG. 19A), cycle 1 Cₘₐₓ (FIG. 19B), and AUCₛₛ (FIG. 19C) for patients in studies PCD4989g (Urothelial Carcinoma Cohort) and IMvigor210 (Cohorts 1 and 2) for atezolizumab Doses 15 mg/kg and 1200 mg q3w. The incidence of AESIs did not show any statistically significant ER relationship with any exposure metric investigated. AUC=area under the concentration-time curve; Cₘₐₓ=maximum concentration in serum; AUCₛₛ=AUC at steady state; AESI=adverse events of special interest; N=number of patients; p=p value of Wald test in logistic regression of incidence versus exposure; q3w=every three weeks. The thick solid line and shaded area represent the logistic regression slope model and 95% prediction interval. The filled circles and error bar represent the incidence in exposure quartiles and 95% CI. The vertical lines are the limits of the exposure quartiles. The cross is AE events (0: No, 1: Yes). The triangle and two-headed arrow represent the mean exposure and exposure interval between the 10^{th} and the 90^{th} percentile for patients receiving 1200 mg atezolizumab, respectively.

FIGS. 20A-20B provide the logistic regression of the proportion of patients experiencing AESI versus atezolizumab exposure metrics cycle 1 AUC (FIG. 20A), and cycle 1 Cₘₐₓ (FIG. 20B) for patients in study IMvigor211 receiving atezolizumab 1200 mg q3w. The analysis of the incidence of AESIs did not show any statistically significant ER relationship with any exposure metric investigated. AUC=area under the concentration-time curve; Cₘₐₓ=maximum concentration in serum; AESI=adverse events of special interest; N=number of patients; p=p value of Wald test in logistic regression of incidence versus exposure; q3w=every three weeks. The thick solid line and shaded area represent the logistic regression slope model and 95% prediction interval. The filled circles and error bar represent the incidence in exposure quartiles and 95% CI. The vertical lines are the limits of the exposure quartiles. The cross is AE events (0: No, 1: Yes). The triangle and two-headed arrow represent the mean exposure and exposure interval between the 10^{th} and the 90^{th} percentile for patients receiving 1200 mg atezolizumab, respectively.

FIGS. 21A-21C provide the logistic regression of the proportion of patients experiencing AE of Grade ≥3 versus atezolizumab exposure metrics cycle 1 AUC (FIG. 21A), cycle 1 Cₘ (FIG. 21B), and AUCₛₛ (FIG. 21C) for patients with NSCLC in studies PCD4989 (NSCLC cohort), BIRCH, POPLAR, and FIR for atezolizumab doses 1 mg/kg to 20 mg/kg, including the 1200 mg Flat Dose. The analysis of the incidence of AEG35 did not show any statistically significant positive ER relationship with any exposure metric investigated. AUC=area under the concentration-time curve; Cₘ=maximum concentration in serum; AUCₛₛ=AUC at steady state; AE=adverse event; AEG35=adverse events of grade 3 to 5; CI=confidence interval; N=number of patients; NSCLC=non-small cell lung cancer; p=p value of Wald test in logistic regression of incidence versus exposure. The thick solid line and shaded area represent the logistic regression slope model and 95% prediction interval. The filled circles and error bar represent the incidence in exposure quartiles and 95% CI. The vertical lines are the limits of the exposure quartiles. The cross is AE events (0: No, 1: Yes). The triangle and two-headed arrow represent the mean exposure and exposure interval between the 10^{th} and the 90^{th} percentile for patients receiving 1200 mg atezolizumab, respectively.

FIGS. 22A-22C provide the logistic regression of the proportion of patients experiencing AE of Grade ≥3 versus atezolizumab exposure metrics cycle 1 AUC (FIG. 22A), cycle 1 Cₘₐₓ (FIG. 22B), or AUCₛₛ (FIG. 22C) for patients with NSCLC in study OAK receiving atezolizumab 1200 mg q3w. The analysis of the incidence of AEG35 did not show any statistically significant positive ER relationship with any exposure metric investigated. AUC=area under the concentration-time curve; Cₘₐₓ=maximum concentration in serum; AUCₛₛ=AUC at steady state; AE=adverse event; AEG35=adverse events of grade 3 to 5; CI=confidence interval; N=number of patients; NSCLC=non-small cell lung cancer; p=p value of Wald test in logistic regression of incidence versus exposure. The thick solid line and shaded area represent the logistic regression slope model and 95% prediction interval. The filled circles and error bar represent the incidence in exposure quartiles and 95% CI. The vertical lines are the limits of the exposure quartiles. The cross is AE events (0: No, 1: Yes). The triangle and two-headed arrow represent the mean exposure and exposure interval between the 10^{th} and the 90^{th} percentile for patients receiving 1200 mg atezolizumab, respectively.

FIGS. 23A-23C provide the logistic regression of the proportion of patients experiencing AESI versus atezolizumab exposure metrics cycle 1 AUC (FIG. 23A), cycle 1 Cₘₐₓ (FIG. 23B), and AUCₛₛ (FIG. 23C) for patients with NSCLC in studies PCD4989 (NSCLC cohort), BIRCH, POPLAR, and FIR for atezolizumab doses 1 mg/kg to 20 mg/kg, including the 1200 mg Flat Dose. The analysis of the incidence of AESIs of the pooled analysis of NSCLC patients in PCD4989g, BIRCH, POPLAR, and FIR did not show any statistically significant ER relationship with Cycle 1 AUC (FIG. 23A), or Cₘₐₓ (FIG. 23B), but did have a statistically significant relationship with AUCₛₛ (FIG. 23C). AUC=area under the concentration-time curve; AUCₛₛ=area under the concentration-time curve at steady-state; Cₘₐₓ=maximum concentration in serum; AESI=adverse events of special interest of any grade; CI=confidence interval; N=number of patients; NSCLC=non-small cell lung cancer; p=p value of Wald test in logistic regression of incidence versus exposure. The thick solid line and shaded area represent the logistic regression slope model and 95% prediction interval. The filled circles and error bar represent the incidence in exposure quartiles and 95% CI. The vertical lines are the limits of the exposure quartiles. The cross is AE events (0: No, 1: Yes). The triangle and two-headed arrow represent the mean exposure and exposure interval between the 10^{th} and the 90^{th} percentile for patients receiving 1200 mg atezolizumab, respectively.

FIGS. 24A-24C provide the logistic regression of the proportion of patients experiencing AESI versus atezolizumab exposure metrics cycle 1 AUC (FIG. 24A), cycle 1 Cₘₐₓ(FIG. 24B), and AUCₛₛ (FIG. 24C) for patients with NSCLC in study OAK receiving atezolizumab 1200 mg q3w. The analysis of the incidence of AESIs did not show any statistically significant ER relationship with any exposure metric investigated. AUC=area under the concentration-time curve; Cₘₐₓ=maximum concentration in serum; AUCₛₛ=area under the concentration-time curve at steady-state; AESI=adverse events of special interest of any grade; CI=confidence interval; N=number of patients; NSCLC=non-small cell lung cancer; p=p value of Wald test in logistic regression of incidence versus exposure. The thick solid line and shaded area represent the logistic regression slope model and 95% prediction interval. The filled circles and error bar represent the incidence in exposure quartiles and 95% CI. The vertical lines are the limits of the exposure quartiles. The cross is AE events (0: No, 1: Yes). The triangle and two-headed arrow represent the mean exposure and exposure interval between the 10^{th} and the 90^{th} percentile for patients receiving 1200 mg atezolizumab, respectively.

FIGS. 25A-25B provide pooled exposure-response analyses of safety in patients with locally advanced or metastatic NSCLC or UC. Indicated AE frequencies ([a, c]grade ≥3 AEs (FIG. 25A); [b, d]AESIs (FIG. 25B)) are plotted vs AUC cycle 1. For legibility, 2 extreme AUC values (>15,000 g·day/mL) are not displayed on the plots. Wald P values from logistic regression of AE incidence vs exposure are displayed. Grey solid lines and shaded areas represent the logistic regression slope model and 95% PI. Filled circles and error bars represent AE proportion in exposure quartiles and 95% CI; vertical lines are the limits of the exposure quartiles. Cross markings (x) represent AE events (0: no, 1: yes). Triangle and 2-headed arrows represent the mean exposure and exposure interval between the 10th and 90th percentiles, respectively, for patients receiving atezolizumab 1200 mg. Cycle 1 AUC corresponds to the AUC during the first 3 weeks after treatment start and with PK parameters estimated based on cycle 1 data only. AE=adverse event; AESI=adverse event of special interest; AUC=area under the concentration-time curve; Cₘₐₓ=maximum serum atezolizumab concentration; N=number of patients; NSCLC=non-small cell lung cancer; PI=prediction interval; PK=pharmacokinetics; UC=urothelial carcinoma.

FIGS. 26A-26B provide pooled exposure-response analyses of safety in patients with locally advanced or metastatic NSCLC or UC. Indicated AE frequencies ([a, c]grade ≥3 AEs (FIG. 26A); [b, d]AESIs (FIG. 26B)) are plotted vs Cₘₐₓ at cycle 1. For legibility, 2 extreme Cₘₐₓ values (>1500 g/mL) are not displayed on the plots. Wald P values from logistic regression of AE incidence vs exposure are displayed. Grey solid lines and shaded areas represent the logistic regression slope model and 95% PI. Filled circles and error bars represent AE proportion in exposure quartiles and 95% CI; vertical lines are the limits of the exposure quartiles. Cross markings (x) represent AE events (0: no, 1: yes). Triangle and 2-headed arrows represent the mean exposure and exposure interval between the 10th and 90th percentiles, respectively, for patients receiving atezolizumab 1200 mg. Cycle 1 AUC corresponds to the AUC during the first 3 weeks after treatment start and with PK parameters estimated based on cycle 1 data only. AE=adverse event; AESI=adverse event of special interest; AUC=area under the concentration-time curve; Cₘₐₓ=maximum serum atezolizumab concentration; N=number of patients; NSCLC=non-small cell lung cancer; PI=prediction interval; PK=pharmacokinetics; UC=urothelial carcinoma.

FIG. 27 illustrates simulated atezolizumab exposure profiles for the indicated dosing regimens (840-mg q2w, 1200-mg q3w, 1680-mg q4w, and 20-mg/kg q3w). Geometric means are plotted. Shaded areas represent 90% PIs. Line: geometric mean; area: 90% prediction interval (500 patients). The PK profiles are displayed over a 28-day period showing 2 doses for 1200-mg q3w, 20-mg/kg q3w and 840-mg q2w; and 1 dose for 1680-mg q4w. The corresponding predicted Cₘₐₓ and Cₘᵢₙ values at Cycle 1 and at steady state are presented in Table 7. PI=prediction interval; q2w=every 2 weeks; q3w=every 3 weeks; q4w=every 4 weeks.

FIG. 28 shows a histogram of the maximum observed Cₘₐₓ concentration for individual patients receiving 20 mg/kg atezolizumab q3w in study PCD4989g.

FIG. 29 provides prediction-corrected VPC of atezolizumab data in TNBC (IMpassion130) using the phase 1 popPK model. Data are plotted on a semi-log scale. Two population-predicted concentrations <1 μg/mL are not displayed on this plot. n=number of samples; Obs=observed; PI=prediction interval; popPK=population pharmacokinetics; Pred=prediction; sim=simulated; TNBC=triple-negative breast cancer; VPC=visual performance check.

FIG. 30 provides an overall summary of adverse events in patients receiving atezolizumab 1200 mg q3w IV or 20 mg/kg IV q3w (atezolizumab-treated safety evaluable patients). The overall safety profile of atezolizumab given as a 20 mg/kg q3w dose was similar to that observed when given as a fixed 1200 mg q3w dose.

FIG. 31 provides the safety margins based on a repeat-dose toxicity study in cynomolgus monkey. AUC=area under the concentration-time curve; Cₘₐₓ=maximum concentration observed; q2w=every 2 weeks; q3w=every 3 weeks; q4w=every 4 weeks; SS=steady state.

### DETAILED DESCRIPTION

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

As used herein, “in conjunction with” refers to administration of one treatment modality in addition to another treatment modality. As such, “in conjunction with” refers to administration of one treatment modality before, during, or after administration of the other treatment modality to the individual.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

As used herein, “cancer” and “cancerous” refer to or describe the physiological condition in mammals that is typically characterized by unregulated cell growth. Included in this definition are benign and malignant cancers as well as dormant tumors or micrometastases. Examples of cancer include but are not limited to, carcinoma, lymphoma, blastoma, sarcoma, and leukemia. More particular examples of such cancers include but are not limited to squamous cell cancer, lung cancer (including small-cell lung cancer, non-small cell lung cancer, adenocarcinoma of the lung, and squamous carcinoma of the lung), melanoma, renal cell carcinoma, cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Examples of cancer may include primary tumors of any of the above types of cancer or metastatic tumors at a second site derived from any of the above types of cancer.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signalling inhibitors, HDAC inhibitors, proteasome inhibitors, and inhibitors of cancer metabolism. In one embodiment the cytotoxic agent is a taxane. In one embodiment the taxane is paclitaxel or docetaxel. In one embodiment the cytotoxic agent is a platinum agent. In one embodiment the cytotoxic agent is an antagonist of EGFR. In one embodiment the antagonist of EGFR is N-(3-ethynylphenyl)-6,7-bis(2-methoxyethoxy)quinazolin-4-amine (e.g., erlotinib). In one embodiment the cytotoxic agent is a RAF inhibitor. In one embodiment, the RAF inhibitor is a BRAF and/or CRAF inhibitor. In one embodiment the RAF inhibitor is vemurafenib. In one embodiment the cytotoxic agent is a PI3K inhibitor.

“Chemotherapeutic agent” includes compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin 71I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG₁λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Wamer-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-111 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

A “growth inhibitory agent” when used herein refers to a compound or composition which inhibits growth of a cell either in vitro or in vivo. In one embodiment, growth inhibitory agent is growth inhibitory antibody that prevents or reduces proliferation of a cell expressing an antigen to which the antibody binds. In another embodiment, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

A “subject” or an “individual” for purposes of treatment refers to any animal classified as a mammal, including humans, domestic and farm animals, and zoo, sports, or pet animals, such as dogs, horses, cats, cows, etc. Preferably, the mammal is human.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any mammalian species can be assigned to one of two clearly distinct types, called kappa (“x”) and lambda (“k”), based on the amino acid sequences of their constant domains.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “species-dependent antibody” is one which has a stronger binding affinity for an antigen from a first mammalian species than it has for a homologue of that antigen from a second mammalian species. Normally, the species-dependent antibody “binds specifically” to a human antigen (e.g., has a binding affinity (Kd) value of no more than about 1×10-7 M, preferably no more than about 1×10-8 M and preferably no more than about 1×10-9 M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, but preferably is a humanized or human antibody.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat Numbering) |
| H1 | H31-H35 | H26-H35 | H26-H32 | H30-H35 (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

As used herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 M, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A patient who “does not have an effective response” to treatment refers to a patient who does not have any one of extending survival (including overall survival and progression free survival); resulting in an objective response (including a complete response or a partial response); or improving signs or symptoms of cancer.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some embodiments, the sample is a sample obtained from the cancer of an individual (e.g., a tumor sample) that comprises tumor cells and, optionally, tumor-infiltrating immune cells. For example, the sample can be a tumor specimen that is embedded in a paraffin block, or that includes freshly cut, serial unstained sections. In some embodiments, the sample is from a biopsy and includes 50 or more viable tumor cells (e.g., from a core-needle biopsy and optionally embedded in a paraffin block; excisional, incisional, punch, or forceps biopsy; or a tumor tissue resection).

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A cancer or biological sample which “has human effector cells” is one which, in a diagnostic test, has human effector cells present in the sample (e.g., infiltrating human effector cells).

A cancer or biological sample which “has FcR-expressing cells” is one which, in a diagnostic test, has FcR-expressing present in the sample (e.g., infiltrating FcR-expressing cells). In some embodiments, FcR is FcγR. In some embodiments, FcR is an activating FcγR.

### II. Methods of Treatment

Provided herein are methods for treating or delaying progression of cancer in an individual, comprising administering to the individual an anti-PD-L1 antibody of the present disclosure in two or more 4-week or 28-day cycles. In some embodiments, the anti-PD-L1 antibody is administered at a dose of 1680 mg per cycle (e.g., the anti-PD-L1 antibody is administered at a dose of 1680 mg every 4 weeks or every 28 days). In some embodiments, the anti-PD-L1 antibody is atezolizumab.

Provided herein are methods for treating or delaying progression of cancer in an individual, comprising administering to the individual an anti-PD-L1 antibody of the present disclosure in two or more 2-week or 14-day cycles. In some embodiments, the anti-PD-L1 antibody is administered at a dose of 840 mg per cycle (e.g., the anti-PD-L1 antibody is administered at a dose of 840 mg every 2 weeks or every 14 days). In some embodiments, the anti-PD-L1 antibody is atezolizumab.

In some embodiments, the anti-PD-L1 antibody is administered at about day 1 of each of the two or more cycles. In some embodiments, the anti-PD-L1 antibody is administered at day 1 of each of the two or more cycles.

In some embodiments, the anti-PD-L1 antibody is administered at a dose of 1680 mg or 840 mg in each of the two or more cycles.

In some embodiments, a treatment of the present disclosure comprises an induction phase and a maintenance phase (or “maintenance therapy”). As is known in the art, a maintenance phase or maintenance therapy may refer to one or more treatments provided after an induction phase or initial therapy, e.g., to prevent recurrence of a cancer. In some embodiments, a maintenance phase or maintenance therapy may be given over a longer period of time than an induction phase or initial therapy. In some embodiments, a maintenance phase or maintenance therapy may be characterized by fewer side effects or toxicities (e.g., associated with short- and/or long-term use) than an induction phase or initial therapy, allowing for a longer duration of use. In some embodiments, an anti-PD-L1 antibody of the present disclosure may be administered to an individual as part of an induction phase or initial therapy, a maintenance phase or maintenance therapy, or both. In some embodiments, a maintenance phase or maintenance therapy is administering to the individual until disease progression or unacceptable toxicity.

In some embodiment, the method for treating a human patient having a cancer comprises administering to the human patient an induction phase followed by administering to the human patient a maintenance phase. In some embodiments, the method for treating a human patient having a cancer comprises administering to the human patient an induction phase followed by administering one or more additional therapeutic agents, such as one or more of bevacizumab, paclitaxel, and carboplatin.

In some embodiments, an anti-PD-L1 antibody of the present disclosure is administered to an individual in a maintenance phase of treatment. For example, in some embodiments, the methods of the present disclosure comprise administering one or more chemotherapies of the present disclosure (e.g., paclitaxel and carboplatin, or carboplatin and etoposide) to an individual for 4-6 cycles (e.g., 4, 5, or 6 cycles) during an induction phase of treatment, then administering the anti-PD-L1 antibody to the individual during a maintenance phase of treatment, e.g., as described herein. In some embodiments, prior to the maintenance phase of treatment, an anti-PD-L1 antibody of the present disclosure is administered to an individual in an induction phase of treatment.

In some embodiments, an anti-PD-L1 antibody of the present disclosure is administered to an individual in one or more 2-week or 14-day cycles during an induction phase of treatment. In some embodiments, an anti-PD-L1 antibody of the present disclosure is administered to an individual at a dose of 840 mg in one or more 2-week or 14-day cycles during an induction phase of treatment. In some embodiments, an anti-PD-L1 antibody of the present disclosure is administered to an individual at a dose of 840 mg on days 1 and 15 of one or more 4-week or 28-day cycles.

In some embodiments, an anti-PD-L1 antibody of the present disclosure is administered to an individual in one or more 3-week or 21-day cycles during an induction phase of treatment. In some embodiments, an anti-PD-L1 antibody of the present disclosure is administered to an individual at about day 1 in one or more 3-week or 21-day cycles during an induction phase of treatment. In some embodiments, an anti-PD-L1 antibody of the present disclosure is administered to an individual at day 1 in one or more 3-week or 21-day cycles during an induction phase of treatment.

In some embodiments, an anti-PD-L1 antibody of the present disclosure is administered to an individual at a dose of 1200 mg in one or more 3-week or 21-day cycles during an induction phase of treatment. In some embodiments, an anti-PD-L1 antibody of the present disclosure is administered to an individual at a dose of 1200 mg on day 1 in one or more 3-week or 21-day cycles during an induction phase of treatment. In some embodiments, an anti-PD-L1 antibody of the present disclosure is administered to an individual at a dose of 1200 mg during each of one or more 3-week or 21-day cycles in an induction phase of treatment.

In some embodiments according to any of the embodiments described herein, the methods further comprise administering to the individual an anti-PD-L1 antibody of the present disclosure (e.g., atezolizumab) at a dose of 1200 mg in one or more 3-week or 21-day cycles prior to treatment with one or more chemotherapies or other anti-neoplastic drug(s) (e.g., carboplatin and etoposide, or carboplatin, paclitaxel, and bevacizumab).

In some embodiments, an anti-PD-L1 antibody of the present disclosure is administered to an individual in one or more 4-week or 28-day cycles during an induction phase of treatment. In some embodiments, an anti-PD-L1 antibody of the present disclosure is administered to an individual at about day 1 in one or more 4-week or 28-day cycles during an induction phase of treatment. In some embodiments, an anti-PD-L1 antibody of the present disclosure is administered to an individual at day 1 in one or more 4-week or 28-day cycles during an induction phase of treatment.

In some embodiments, an anti-PD-L1 antibody of the present disclosure is administered to an individual at a dose of 1680 mg in one or more 4-week or 28-day cycles during an induction phase of treatment. In some embodiments, an anti-PD-L1 antibody of the present disclosure is administered to an individual at a dose of 1680 mg on day 1 in one or more 4-week or 28-day cycles during an induction phase of treatment. In some embodiments, an anti-PD-L1 antibody of the present disclosure is administered to an individual at a dose of 1680 mg during each of one or more 4-week or 28-day cycles in an induction phase of treatment.

In some embodiments, the anti-PD-L1 antibody (e.g., atezolizumab) is administered to an individual intravenously over 30 (±15 minutes) at a dose of 1680 mg in one or more 4-week or 28-day cycles. In some embodiments, the anti-PD-L1 antibody (e.g., atezolizumab) is administered to an individual intravenously over 30 (±15 minutes) at a dose of 1680 mg on day 1 of one or more 4-week or 28-day cycles. In some embodiments, the anti-PD-L1 antibody (e.g., atezolizumab) is administered to an individual intravenously over 60 (±15 minutes) at a dose of 1680 mg in one or more 4-week or 28-day cycles. In some embodiments, the anti-PD-L1 antibody (e.g., atezolizumab) is administered to an individual intravenously over 60 (±15 minutes) at a dose of 1680 mg on day 1 of one or more 4-week or 28-day cycles. In some embodiments, the anti-PD-L1 antibody (e.g., atezolizumab) is administered to an individual intravenously over 60 (±15 minutes) at a dose of 1680 mg on day 1 of one or more 4-week or 28-day cycles during an induction phase of treatment. In some embodiments, the anti-PD-L1 antibody (e.g., atezolizumab) is administered to an individual intravenously over 60 (±15 minutes) at a dose of 1680 mg on day 1 of one or more 4-week or 28-day cycles during a maintenance phase of treatment.

In some embodiments, the methods may further comprise an additional therapy. In some embodiments, the methods may further comprise administering to the individual an additional therapeutic agent. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional agent comprises a chemotherapeutic agent. In some embodiments, the chemotherapeutic agent is standard of care for the cancer to be treated. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation.

In some embodiments, the additional therapy comprises a taxane. In some embodiments, the additional therapy is administered during an induction phase of treatment. Taxanes (e.g., paclitaxel and docetaxel) are widely prescribed anticancer drugs initially derived from the yew tree. Taxanes promote the assembly of microtubules from tubulin dimers and stabilize microtubules by preventing depolymerization, which results in the inhibition of mitosis and cellular death. Docetaxel is a semisynthetic analog of paclitaxel.

Paclitaxel is an exemplary taxane used in the methods described herein. The drug substance, TAXOL®, has the chemical name 5β,20-Epoxy-1,2α,4,7β,10β,13α-hexahydroxytax-11-en-9-one 4,10-diacetate 2-benzoate 13-ester with (2R,3S)—N-benzoyl-3-phenylisoserine with a molecular formula of C₄₇H₅₁NO₁₄ and a molecular weight of 853.9. References to taxanes such as paclitaxel herein also include conjugates thereof, such as nab-paclitaxel, an albumin-bound form of paclitaxel marketed as ABRAXANE®.

Paclitaxel has the following chemical structure:

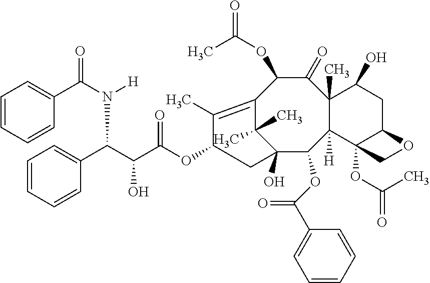

Paclitaxel is commercially available as TAXOL®, ABRAXANE®, XYTOTAX®, OPAXIO®, GENEXOL-PM®, TAXOPREXIN®, and others. Docetaxel is commercially available as TAXOTERE®, JEVTANA®, and others.

In some embodiments, the additional therapy comprises a topoisomerase II inhibitor. In some embodiments, the additional therapy is administered during an induction phase of treatment. Inhibitors of topoisomerase II (e.g., etoposide (VP-16), teniposide, doxorubicin, daunorubicin, mitoxantrone, amsacrine, ellipticines, aurintricarboxylic acid, and HU-331) are also widely used antitumor drugs that stabilize topoisomerase II:DNA covalent complexes (i.e., “cleavage complexes”) following the formation of enzyme-mediated DNA breaks. The accumulation of such cleavage complexes induces cell death pathways.

Etoposide is an exemplary topoisomerase II inhibitor used in the methods described herein. Etoposide is typically administered as the prodrug etoposide phosphate, the chemical name for which is: 4′-Demethylepipodophyllotoxin 9-[4,6-O—(R)-ethylidene-β-Dglucopyranoside], 4′ (dihydrogen phosphate).

Etoposide phosphate has the following structure:

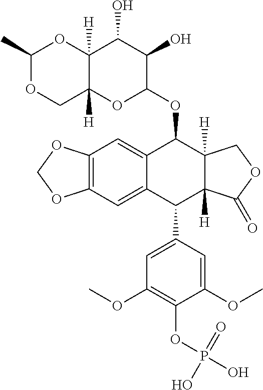

Etoposide phosphate, a phosphate ester of etoposide, is a semi-synthetic derivative of podophyllotoxin and is converted to etoposide by dephosphorylation. Etoposide causes the induction of DNA strand breaks by an interaction with DNA-topoisomerase II or the formation of free radicals, leading to cell cycle arrest (primarily at the G2 stage of the cell cycle) and cell death. Etoposide is commercially available as ETOPOPHOS®, TOPOSAR™, VP-16, VEPESID®, ACTITOP, ASIDE, BIOPOSIDE, CTOP, CYTOP, EPOSED, ESIDE, ETHOPUL, ETOLON, ETONIS, ETOPLAST, ETOSID, ETOVEL, FYTOP, FYTOSID, LASTET, NZYTOP, ONCOSIDE, PLACID, POSID, RETOPSON, TEVASIDE, TOPOK, TOPOSIDE, and others.

In some embodiments, the additional therapy comprises an antimetabolite. In some embodiments, the additional therapy is administered during an induction phase of treatment. Antimetabolites (e.g., pemetrexed, 5-fluorouracil, 6-mercaptopurine, capecitabine, cytarabine, floxuridine, fludarabine, hydroxycarbamide, methotrexade, and others) are widely used antitumor drugs that interfere with one or more enzymes necessary for DNA synthesis. Antimetabolites typically act by a variety of mechanisms including, e.g., incorporation into nucleic acids, thereby triggering apoptosis, or, e.g., competition for binding sites of enzymes involved in nucleotide synthesis, thereby depleting the supply required for DNA and/or RNA replication and cell proliferation.

Pemetrexed is an exemplary antimetabolite used in the methods described herein. Pemetrexed is a folic acid analogue. The drug substance, pemetrexed disodium heptahydrate, has the chemical name L-glutamic acid, N-[4-[2-(2-amino-4,7-dihydro-4-oxo-1H-pyrrolo[2,3-d]pyrimidin-5yl)ethyl]benzoyl]-, disodium salt, heptahydrate with a molecular formula of C₂₀H₁₉N₅Na₂O₆·7H₂O and a molecular weight of 597.49.

Pemetrexed disodium heptahydrate has the following structure:

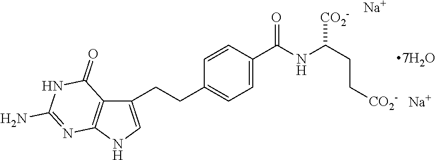

Pemetrexed inhibits multiple folate-dependent enzymes used in thymine and purine synthesis, namely, thymidylate synthase (TS), dihydrofolate reductase (DHFR), and glycinamide ribonucleotide formyltransferase (GARFT) (see Shih et al. (1997) Cancer Res. 57:1116-23). By inhibiting the formation of precursor purine and pyrimidine nucleotides, pemetrexed prevents the formation of DNA and RNA, which are required for the growth and survival of both normal cells and cancer cells. Pemetrexed is commercially available as ALIMTA®, GIOPEM, PEXATE, PEMANAT, PEMEX, PEMMET, PEXATE, RELITREXED, TEMERAN, CIAMBRA, and others.

In some embodiments, the additional therapy comprises a VEGF antagonist, e.g., an anti-VEGF antibody. In some embodiments, the additional therapy is administered during an induction phase of treatment and/or during a maintenance phase of treatment. In some embodiments, the anti-VEGF antibody may be a human or humanized antibody. In some embodiments, the anti-VEGF antibody may be a monoclonal antibody. Other examples of VEGF antagonists include, without limitation, a soluble VEGF receptor or a soluble VEGF receptor fragment that specifically binds to VEGF, a VEGF receptor molecule or VEGF binding fragment thereof (e.g., a soluble form of a VEGF receptor), and a chimeric VEGF receptor protein.

The VEGF antigen to be used for production of VEGF antibodies may be, e.g., the VEGF₁₆₅ molecule as well as other isoforms of VEGF or a fragment thereof containing the desired epitope. In one embodiment, the desired epitope is the one recognized by bevacizumab, which binds to the same epitope as the monoclonal anti-VEGF antibody A4.6.1 produced by hybridoma ATCC HB 10709 (known as “epitope A.4.6.1” defined herein). Other forms of VEGF useful for generating anti-VEGF antibodies of the invention will be apparent to those skilled in the art.

Anti-VEGF antibodies that are useful in the methods of the invention include any antibody, or antigen binding fragment thereof, that bind with sufficient affinity and specificity to VEGF and can reduce or inhibit the biological activity of VEGF. An anti-VEGF antibody will usually not bind to other VEGF homologues such as VEGF-B or VEGF-C, nor other growth factors such as P1GF, PDGF, or bFGF.

In certain embodiments, the anti-VEGF antibodies include, but are not limited to, a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. (1997) Cancer Res. 57:4593-4599. In one embodiment, the anti-VEGF antibody is “bevacizumab (BV)”, also known as “rhuMAb VEGF” or “AVASTIN®”. It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1.

In some embodiments, the anti-VEGF antibody is bevacizumab. Bevacizumab (AVASTIN®) was the first anti-angiogenesis therapy approved by the FDA and is approved for the treatment metastatic colorectal cancer (first- and second-line treatment in combination with intravenous 5-FU-based chemotherapy), advanced non-squamous, non-small cell lung cancer (NSCLC) (first-line treatment of unresectable, locally advanced, recurrent or metastatic NSCLC in combination with carboplatin and paclitaxel) and metastatic HER2-negative breast cancer (previously untreated, metastatic HER2-negative breast cancer in combination with paclitaxel.

Bevacizumab and other humanized anti-VEGF antibodies are further described in U.S. Pat. No. 6,884,879 issued Feb. 26, 2005. Additional antibodies include the G6 or B20 series antibodies (e.g., G6-31, B20-4.1), as described in PCT Publication No. WO2005/012359, PCT Publication No. WO2005/044853, and U.S. Patent Application 60/991,302, the content of these patent applications are expressly incorporated herein by reference. For additional antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., Journal of Immunological Methods 288:149-164 (2004). Other antibodies include those that bind to a functional epitope on human VEGF comprising of residues F17, M18, D19, Y21, Y25, Q89, 1191, K101, E103, and C104 or, alternatively, comprising residues F17, Y21, Q22, Y25, D63, 183 and Q89.

In one embodiment of the invention, the anti-VEGF antibody has a light chain variable region comprising the following amino acid sequence: DIQMTQSPSS LSASVGDRVT ITCSASQDIS NYLNWYQQKP GKAPKVLIYF TSSLHSGVPS RFSGSGSGTD FTLTISSLQP EDFATYYCQQ YSTVPWTFGQ GTKVEIKR. (SEQ ID NO:11); and/or a heavy chain variable region comprising the following amino acid sequence: EVQLVESGGG LVQPGGSLRL SCAASGYTFT NYGMNWVRQA PGKGLEWVGW INTYTGEPTY AADFKRRFTF SLDTSKSTAY LQMNSLRAED TAVYYCAKYP HYYGSSHWYF DVWGQGTLVT VSS (SEQ ID NO:12).

In some embodiments, the anti-VEGF antibody comprises one, two, three, four, five, or six hypervariable region (HVR) sequences of bevacizumab. In some embodiments, the anti-VEGF antibody comprises one, two, three, four, five, or six hypervariable region (HVR) sequences of selected from (a) HVR-H1 comprising the amino acid sequence of GYTFTNYGMN (SEQ ID NO:13); (b) HVR-H2 comprising the amino acid sequence of WINTYTGEPTYAADFKR (SEQ ID NO:14); (c) HVR-H3 comprising the amino acid sequence of YPHYYGSSHWYFDV (SEQ ID NO:19); (d) HVR-L1 comprising the amino acid sequence of SASQDISNYLN (SEQ ID NO:20); (e) HVR-L2 comprising the amino acid sequence of FTSSLHS (SEQ ID NO:21); and (f) HVR-L3 comprising the amino acid sequence of QQYSTVPWT (SEQ ID NO:22). In some embodiments, the anti-VEGF antibody comprises one, two, three, four, five, or six hypervariable region (HVR) sequences of an antibody described in U.S. Pat. No. 6,884,879. In some embodiments, the anti-VEGF antibody comprises one, two, or three hypervariable region (HVR) sequences of a light chain variable region comprising the following amino acid sequence: DIQMTQSPSS LSASVGDRVT ITCSASQDIS NYLNWYQQKP GKAPKVLIYF TSSLHSGVPS RFSGSGSGTD FTLTISSLQP EDFATYYCQQ YSTVPWTFGQ GTKVEIKR. (SEQ ID NO:11) and/or one, two, or three hypervariable region (HVR) sequences of a heavy chain variable region comprising the following amino acid sequence: EVQLVESGGG LVQPGGSLRL SCAASGYTFT NYGMNWVRQA PGKGLEWVGW INTYTGEPTY AADFKRRFTF SLDTSKSTAY LQMNSLRAED TAVYYCAKYP HYYGSSHWYF DVWGQGTLVT VSS (SEQ ID NO:12).

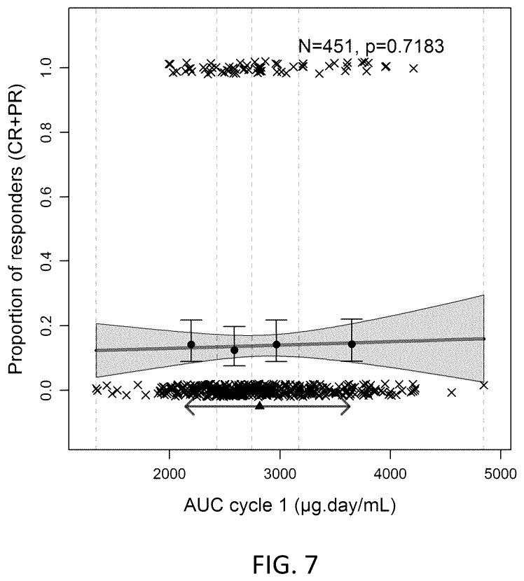

A “G6 series antibody” is an anti-VEGF antibody that is derived from a sequence of a G6 antibody or G6-derived antibody according to any one of FIGS. 7, 24-26, and 34-35 of PCT Publication No. WO2005/012359, the entire disclosure of which is expressly incorporated herein by reference. See also PCT Publication No. WO2005/044853, the entire disclosure of which is expressly incorporated herein by reference. In one embodiment, the G6 series antibody binds to a functional epitope on human VEGF comprising residues F17, Y21, Q22, Y25, D63, 183 and Q89.

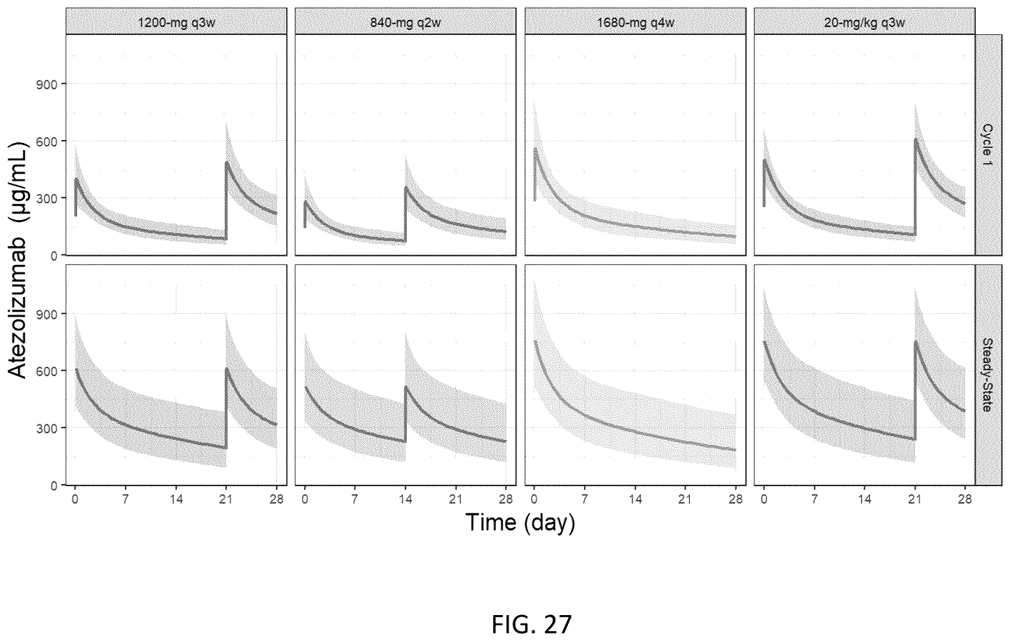

A “B20 series antibody” is an anti-VEGF antibody that is derived from a sequence of the B20 antibody or a B20-derived antibody according to any one of FIGS. 27-29 of PCT Publication No. WO2005/012359, the entire disclosure of which is expressly incorporated herein by reference. See also PCT Publication No. WO2005/044853, and U.S. Patent Application 60/991,302, the content of these patent applications are expressly incorporated herein by reference. In one embodiment, the B20 series antibody binds to a functional epitope on human VEGF comprising residues F17, M18, D19, Y21, Y25, Q89, 191, K101, E103, and C104.

A “functional epitope” (when used in reference to a VEGF epitope) refers to amino acid residues of an antigen that contribute energetically to the binding of an antibody. Mutation of any one of the energetically contributing residues of the antigen (for example, mutation of wild-type VEGF by alanine or homolog mutation) will disrupt the binding of the antibody such that the relative affinity ratio (IC50mutant VEGF/IC50wild-type VEGF) of the antibody will be greater than 5 (see Example 2 of WO2005/012359). In one embodiment, the relative affinity ratio is determined by a solution binding phage displaying ELISA. Briefly, 96-well Maxisorp immunoplates (NUNC) are coated overnight at 4° C. with an Fab form of the antibody to be tested at a concentration of 2 μg/ml in PBS, and blocked with PBS, 0.5% BSA, and 0.05% Tween20 (PBT) for 2 h at room temperature. Serial dilutions of phage displaying hVEGF alanine point mutants (residues 8-109 form) or wild type hVEGF (8-109) in PBT are first incubated on the Fab-coated plates for 15 min at room temperature, and the plates are washed with PBS, 0.05% Tween20 (PBST). The bound phage is detected with an anti-M13 monoclonal antibody horseradish peroxidase (Amersham Pharmacia) conjugate diluted 1:5000 in PBT, developed with 3,3′,5,5′-tetramethylbenzidine (TMB, Kirkegaard & Perry Labs, Gaithersburg, Md.) substrate for approximately 5 min, quenched with 1.0 M H3PO4, and read spectrophotometrically at 450 nm. The ratio of IC50 values (IC50,ala/IC50,wt) represents the fold of reduction in binding affinity (the relative binding affinity).

In some embodiments, the additional therapy comprises a platinum agent or platinum-containing chemotherapy. In some embodiments, the additional therapy is administered during an induction phase of treatment. Platinum agents/platinum-containing chemotherapies (such as, e.g., cisplatin, carboplatin, oxaliplatin, and staraplatin) are widely used antitumor drugs that cause crosslinking of DNA as monoadduct, interstrand crosslinks, intrastrand crosslinks or DNA protein crosslinks. Platinum agents typically act on the adjacent N-7 position of guanine, forming a 1, 2 intrastrand crosslink (Poklar et al. (1996). Proc. Natl. Acad. Sci. U.S.A. 93 (15): 7606-11; Rudd et al. (1995). Cancer Chemother. Pharmacol. 35 (4): 323-6). The resultant crosslinking inhibits DNA repair and/or DNA synthesis in cancer cells.

Carboplatin is an exemplary platinum coordination compound used in the methods described herein. The chemical name for carboplatin is platinum, diammine[1,1-cyclobutanedicarboxylato(2-)-O,O′]—, (SP-4-2), and carboplatin has the following structural formula:

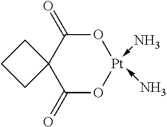

Carboplatin is a crystalline powder with the molecular formula of C₆H₁₂N₂O₄Pt and a molecular weight of 371.25. It is soluble in water at a rate of approximately 14 mg/mL, and the pH of a 1% solution is 5 to 7. It is virtually insoluble in ethanol, acetone, and dimethylacetamide. Carboplatin produces predominantly interstrand DNA cross-links, and this effect is cell-cycle nonspecific. Carboplatin is commercially available as PARAPLATIN®, BIOCARN, BLASTOCARB, BLASTOPLATIN, CARBOKEM, CARBOMAX, CARBOPA, CARBOPLAN, CARBOTEEN, CARBOTINAL, CYTOCARB, DUCARB, KARPLAT, KEMOCARB, NAPROPLAT, NEOPLATIN, NISCARBO, ONCOCARBIN, TEVACARB, WOMASTIN, and others.

Cisplatin is another exemplary platinum coordination compound used in the methods described herein. The chemical name for cisplatin is dichloroplatinum diammoniate, and cisplatin has the following structural formula:

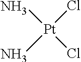

Cisplatin is an inorganic and water-soluble platinum complex with the molecular formula of Pt(NH₃)₂Cl₂ and a molecular weight of 300.046. After undergoing hydrolysis, it reacts with DNA to produce both intra and interstrand crosslinks. These crosslinks appear to impair replication and transcription of DNA. The cytotoxicity of cisplatin correlates with cellular arrest in the G2 phase of the cell cycle. Cisplatin is commercially available as PLATINOL®, PLATINOL®-AQ, CDDP, CISPLAN, CISPLAT, PLATIKEM, PLATIONCO, PRACTICIS, PLATICIS, BLASTOLEM, CISMAX, CISPLAN, CISPLATINUM, CISTEEN, DUPLAT, KEMOPLAT, ONCOPLATIN-AQ, PLATINEX, PLATIN, TEVAPLATIN, and others.

In some embodiments, an additional therapy or agent is administered to the individual during an induction phase of treatment. In some embodiments, an additional therapy or agent is administered to the individual during a maintenance phase of treatment. For example, in some embodiments, an antibody is administered to the individual during a maintenance phase of treatment.

In some embodiments, prior to treatment using a method described herein, the individual has been treated with a platinum-containing chemotherapy, e.g., as described supra. In some embodiments, the individual is ineligible for a platinum-containing chemotherapy, e.g., as described supra.

In some embodiments, prior to treatment using a method described herein, the individual has been treated with an adjuvant or neoadjuvant chemotherapy. In some embodiments, the cancer is locally advanced or metastatic non-small cell lung cancer, and the individual has been treated with a chemotherapy prior to treatment using a method described herein.

In some embodiments, a sample from the cancer of the individual comprises tumor-infiltrating immune cells that express PD-L1. In some embodiments, a sample from the cancer of the individual comprises tumor-infiltrating immune cells that express PD-L1 and cover 1% or more of the tumor area. In some embodiments, tumor-infiltrating immune cells that express PD-L1 are assayed via immunohistochemical assay, e.g., the VENTANA SP142 assay.

In some embodiments, the individual is “PD-L1 high.” In some embodiments, a patient is “PD-L1 high” if tumor cells expressing PD-L1 in a pre-treatment sample from the patient total ≥50% of the total tumor cells in the sample. In some embodiments, PD-L1 expression on ≥50% of the tumor cells in a pretreatment sample is defined/scored as “TC3.” In some embodiments a patient is “PD-L1 high” if tumor-infiltrating immune cells expressing PD-L1 in a pre-treatment sample from the patient total ≥10% of the total tumor-filtrating immune cells in the sample. In some embodiments, PD-L1 expression on ≥10% of the tumor-infiltrating immune cells in a pretreatment sample is defined/scored as “IC3.” In some embodiments, the pre-treatment sample is a fresh tumor sample. In some embodiments, the pre-treatment sample is a formalin-fixed paraffin-embedded (FFPE) tumor sample. In some embodiments, PD-L1 expression level on the tumor cells and/or the tumor-infiltrating immune cells in the pre-treatment sample is determined via immunohistochemical assay. In some embodiments, the immunohistochemical assay is the VENTANA SP142 assay.

In some embodiments, a patient is “PD-L1 low” if tumor cells expressing PD-L1 in a pre-treatment sample from the patient total 1% to <5% of the total tumor cells in the sample. In some embodiments, PD-L1 expression on 1% to <5% of the tumor cells in a pretreatment sample is defined/scored as “TC1.” In some embodiments, a patient is “PD-L1 low” if tumor cells expressing PD-L1 in a pre-treatment sample from the patient total 5% to <50% of the total tumor cells in the sample. In some embodiments, PD-L1 expression on 5% to <50% of the tumor cells in a pretreatment sample is defined/scored as “TC2.” In some embodiments a patient is “PD-L1 low” if tumor-infiltrating immune cells expressing PD-L1 in a pre-treatment sample from the patient total 1% to <5% of of the total tumor-filtrating immune cells in the sample. In some embodiments, PD-L1 expression on 1% to <5% of the tumor-infiltrating immune cells in a pretreatment sample is defined/scored as “IC1.” In some embodiments a patient is “PD-L1 low” if tumor-infiltrating immune cells expressing PD-L1 in a pre-treatment sample from the patient total 5% to <10% of of the total tumor-filtrating immune cells in the sample. In some embodiments, PD-L1 expression on 5% to <10% of the tumor-infiltrating immune cells in a pretreatment sample is defined/scored as “IC2.” In some embodiments, the pre-treatment sample is a fresh tumor sample. In some embodiments, the pre-treatment sample is a formalin-fixed paraffin-embedded (FFPE) tumor sample. In some embodiments, PD-L1 expression level on the tumor cells and/or the tumor-infiltrating immune cells in the pre-treatment sample is determined via immunohistochemical assay. In some embodiments, the immunohistochemical assay is the VENTANA SP142 assay.

In some embodiments, the individual is “PD-L1 negative.” In some embodiments, a patient is “PD-L1 negative” if tumor cells expressing PD-L1 in a pre-treatment sample from the patient total <1% of the total tumor cells in the sample. In some embodiments, PD-L1 expression on <1% of the tumor cells in a pretreatment sample is defined as “TC0.” In some embodiments a patient is “PD-L1 negative” if tumor-infiltrating immune cells expressing PD-L1 in a pre-treatment sample from the patient total <1% of the total tumor-filtrating immune cells in the sample. In some embodiments, PD-L1 expression on <1% of the tumor-infiltrating immune cells in a pretreatment sample is defined as “IC0.” In some embodiments, the pre-treatment sample is a fresh tumor sample. In some embodiments, the pre-treatment sample is a formalin-fixed paraffin-embedded (FFPE) tumor sample. In some embodiments, PD-L1 expression level in the tumor cells and/or the tumor-infiltrating immune cells in the pre-treatment sample is determined via immunohistochemical assay. In some embodiments, the immunohistochemical assay is the VENTANA SP142 assay.

In some embodiments, TC0, TC1, TC2, TC3, IC0, IC1, IC2, and IC3 are defined/scored as summarized in the tables below:

Exemplary tumor cell (TC) and tumor-infiltrating
immune cell (IC) scoring definitions

| Score | Percentage of PD-L1-expressing cells |
|---|---|
| TC3 or IC3 | ≥50% of TC or ≥10% of IC |
| TC2/3 or IC2/3 | ≥5% of TC or IC |
| TC1/2/3 or IC1/2/3 | ≥1% of TC or IC |
| TC1/2 or IC1/2 | ≥1% of TC or IC and <50% of TC or <10% of IC |
| TC0/1/2 and IC0/1/2 | <50% of TC and <10% of IC |
| TC0 and IC0 | <1% of TC and IC |

IC, tumour-infiltrating immune cell;
PD-L1, programmed death-ligand 1;
TC, tumour cell.
From Socinski M, et al. the N Engl filled. Atezolizumab for first-line treatment of metastatic nonsquamous NSCLC. 2018; 378: 2288-301.

In another aspect, the individual has cancer that expresses (has been shown to express, e.g., in a diagnostic test) a PD-L1 biomarker. In some embodiments, the patient's cancer expresses low PD-L1 biomarker. In some embodiments, the patient's cancer expresses high PD-L1 biomarker. In some embodiments of any of the methods, assays and/or kits, the PD-L1 biomarker is absent from the sample when it comprises 0% of the sample.

In some embodiments, provided herein are methods for treating a human patient having locally advanced or metastatic urothelial carcinoma, wherein the human patient is not eligible for cisplatin-containing chemotherapy and whose tumor(s) express PD-L1 (PD-L1 stained tumor-infiltrating immune cells [IC]covering >5% of the tumor area), as determined by an FDA-approved test. In some embodiments, provided herein are methods for treating a human patient having locally advanced or metastatic urothelial carcinoma, wherein the human patient is is not eligible for any platinum-containing chemotherapy regardless of PD-L1 status. In some embodiments, provided herein are methods for treating a human patient having locally advanced or metastatic urothelial carcinoma, wherein the human patient has disease progression during or following any platinum-containing chemotherapy, or within 12 months of neoadjuvant or adjuvant chemotherapy.

In some embodiments, provided herein are methods for treating a human patient having locally advance or metastatic urothelial carcinoma, wherein the method comprises administering an anti-PD-L1 antibody to the human patient after a prior platinum-containing chemotherapy. In some embodiments, provided herein are methods for treating a human patient having locally advance or metastatic urothelial carcinoma, wherein the method comprises administering an anti-PD-L1 antibody to the human patient, and wherein the human patient is considered cisplatin ineligible, and whose tumours have a PD-L1 expression >5%. In some embodiments, the human patient is an adult.

In some embodiments, provided herein are methods for treating a human patient having metastatic non-small cell lung cancer with no EGFR or ALK genomic tumor aberrations. In some embodiments, the method comprises administering to the human patient an anti-PD-L1 antibody in combination with bevacizumab, paclitaxel, and carboplatin.

In some embodiments, provided herein are methods for treating a human patient having metastatic non-small cell lung cancer having a EGFR and/or ALK genomic tumor aberration, wherein the method comprises administering to the human patient an anti-PD-L1 antibody in combination with bevacizumab, paclitaxel, and carboplatin, wherein the human patient failed a targeted therapy for a non-small cell lung cancer.

In some embodiments, provided herein are methods for treating a human patient having metastatic non-small cell lung cancer, and wherein the human patient progressed during or following platinum-containing chemotherapy. In some embodiments, the method comprises administering to the human patient an anti-PD-L1 antibody as a single agent. In some embodiments, wherein the human patient has an EGFR or ALK genomic tumor aberrations, the patient has progressed on a targeted therapy. In some embodiments, wherein the human patient has an EGFR or ALK genomic tumor aberrations, the patient has progressed on an FDA-approved therapy.

In some embodiments, provided herein are methods for treating a human patient having locally advanced or metastatic non-small cell lung cancer, wherein the method comprises administering to the human patient an anti-PD-L1 antibody after prior chemotherapy.

In some embodiments, provided herein are methods for treating a human patient having locally advanced or metastatic triple-negative breast cancer. In some embodiments, the cancer is unresectable locally advanced or metastatic triple-negative breast cancer. In some embodiments, the tumor expresses PD-L1 (PD-L1 stained tumor-infiltrating immune cells [IC] of any intensity covering >1% of the tumor area), as determined by an FDA-approved test. In some embodiments, the method comprises administering to the human patient an anti-PD-L1 antibody in combination with paclitaxel protein-bound.

In some embodiments of any of the methods, assays and/or kits, the PD-L1 biomarker is present in the sample when it comprises more than 0% of the sample. In some embodiments, the PD-L1 biomarker is present in at least 1% of the sample. In some embodiments, the PD-L1 biomarker is present in at least 5% of the sample. In some embodiments, the PD-L1 biomarker is present in at least 10% of the sample.

In some embodiments of any of the methods, assays and/or kits, the PD-L1 biomarker is detected in the sample using a method selected from the group consisting of FACS, Western blot, ELISA, immunoprecipitation, immunohistochemistry, immunofluorescence, radioimmunoassay, dot blotting, immunodetection methods, HPLC, surface plasmon resonance, optical spectroscopy, mass spectrometry, HPLC, qPCR, RT-qPCR, multiplex qPCR or RT-qPCR, RNA-seq, microarray analysis, SAGE, MassARRAY technique, and FISH, and combinations thereof.

In some embodiments of any of the methods, assays and/or kits, the PD-L1 biomarker is detected in the sample by protein expression. In some embodiments, protein expression is determined by immunohistochemistry (IHC). In some embodiments, the PD-L1 biomarker is detected using an anti-PD-L1 antibody. In some embodiments, the PD-L1 biomarker is detected as a weak staining intensity by IHC. In some embodiments, the PD-L1 biomarker is detected as a moderate staining intensity by IHC. In some embodiments, the PD-L1 biomarker is detected as a strong staining intensity by IHC. In some embodiments, the PD-L1 biomarker is detected on tumor cells, tumor infiltrating immune cells, stromal cells and any combinations thereof. In some embodiments, the staining is membrane staining, cytoplasmic staining or combinations thereof. In some embodiments, the immunohistochemical assay is the VENTANA SP142 assay.

In some embodiments of any of the methods, assays and/or kits, the absence of the PD-L1 biomarker is detected as absent or no staining in the sample. In some embodiments of any of the methods, assays and/or kits, the presence of the PD-L1 biomarker is detected as any staining in the sample.

In some embodiments according to any of the embodiments described herein, the individual is human.

In some embodiments, the anti-PD-L1 antibody is administered intravenously, intramuscularly, subcutaneously, topically, orally, transdermally, intraperitoneally, intraorbitally, by implantation, by inhalation, intrathecally, intraventricularly, or intranasally. In some embodiments, the anti-PD-L1 antibody is administered by intravenous infusion. In some embodiments, the anti-PD-L1 antibody is administered by intravenous infusion over 30 minutes or over 60 minutes. In some embodiments, a first dose of the anti-PD-L1 antibody is administered by intravenous infusion over 60 minutes, and subsequent dose(s) of the anti-PD-L1 antibody are administered by intravenous infusion over 30 minutes (e.g., if the first dose is tolerated).

In some embodiments according to any of the embodiments described herein, a cancer to be treated by the methods of the present disclosure includes, but is not limited to, colorectal cancer, renal cell cancer (e.g., renal cell carcinoma), melanoma, bladder cancer, ovarian cancer, breast cancer (e.g., triple-negative breast cancer, HER2-positive breast cancer, or hormone receptor-positive cancer), and non-small-cell lung cancer (e.g., squamous non-small-cell lung cancer or non-squamous non-small-cell lung cancer). In some embodiments, a cancer to be treated by the methods of the present disclosure includes, but is not limited to, a carcinoma, lymphoma, blastoma, sarcoma, and leukemia. In some embodiments, a cancer to be treated by the methods of the present disclosure includes, but is not limited to, squamous cell cancer, lung cancer (including small-cell lung cancer, non-small cell lung cancer, adenocarcinoma of the lung, and squamous carcinoma of the lung), melanoma, renal cell carcinoma, cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. In some embodiments, the cancer may be an early stage cancer or a late stage cancer. In some embodiments, the cancer may be a primary tumor. In some embodiments, the cancer may be a metastatic tumor at a second site derived from any of the above types of cancer.

In some embodiments, a cancer to be treated by the methods of the present disclosure is selected from the group consisting of breast cancer, colorectal cancer, lung cancer, renal cell carcinoma (RCC), ovarian cancer, melanoma, and bladder cancer. In some embodiments, the breast cancer is triple-negative breast cancer, e.g., the cancer is estrogen receptor-negative (ER-negative), progesterone receptor-negative (PR-negative), and HER2-negative. In some embodiments, the lung cancer is non-small cell lung cancer (NSCLC). In some embodiments, the lung cancer is small cell lung cancer (SCLC). In some embodiments, the bladder cancer is urothelial carcinoma.

In some embodiments, the cancer is locally advanced or metastatic.

In some embodiments, the cancer is locally advanced or metastatic urothelial carcinoma. In some embodiments, the cancer is locally advanced or metastatic urothelial carcinoma, and prior to treatment using a method described herein, the individual has been treated with a platinum-containing chemotherapy. In some embodiments, the cancer is locally advanced or metastatic urothelial carcinoma, and the individual is ineligible for a platinum-containing chemotherapy. In some embodiments, the cancer is locally advanced or metastatic urothelial carcinoma, the individual is ineligible for a platinum-containing chemotherapy (e.g., containing cisplatin), and the cancer expresses PD-L1 (e.g., a sample obtained from the cancer shows PD-L1-expressing tumor-infiltrating immune cells covering 5% or more of the tumor area, which can be determined, e.g., using an immunohistochemical assay). In some embodiments, the cancer is locally advanced or metastatic urothelial carcinoma, and, prior to treatment using a method described herein, the individual has had disease progression during or following treatment with a platinum-containing chemotherapy. In some embodiments, the cancer is locally advanced or metastatic urothelial carcinoma, and, prior to treatment using a method described herein, the individual has had disease progression within 12 months of treatment with a neoadjuvant or adjuvant chemotherapy.

In some embodiments, the cancer is NSCLC. In some embodiments, the cancer is metastatic non-squamous NSCLC. In some embodiments, the cancer is NSCLC without an EGFR or ALK genomic tumor aberration or mutation. In some embodiments, the cancer is NSCLC (e.g., metastatic non-squamous NSCLC) without an EGFR or ALK genomic tumor aberration or mutation, and the methods further comprise administering an anti-VEGF antibody (e.g., bevacizumab), taxane (e.g., paclitaxel or protein-bound paclitaxel), and platinum-containing chemotherapy (e.g., carboplatin) in combination with the anti-PD-L1 antibody (e.g., atezolizumab).

In some embodiments, the cancer is locally advanced or metastatic NSCLC. In some embodiments, the cancer is locally advanced or metastatic NSCLC, and prior to treatment using a method described herein, the individual has been treated with a chemotherapy. In some embodiments, the cancer is locally advanced or metastatic NSCLC, the cancer has an EGFR activating or ALK-positive mutation, and prior to treatment using a method described herein, the individual has been treated with a targeted therapy. In some embodiments, the cancer is locally advanced or metastatic NSCLC, the cancer has an EGFR activating or ALK-positive mutation, and prior to treatment using a method described herein, the individual has had disease progression on treatment with a targeted therapy. In some embodiments, the cancer is locally advanced or metastatic NSCLC, and, prior to treatment using a method described herein, the individual has had disease progression during or following treatment with a platinum-containing chemotherapy.

Various activating EGFR mutations are known in the art. The EGFR gene encodes the epidermal growth factor receptor, also known as v-ERB-B, ERBB, ERBB1, HER1, and SA7. In some embodiments, the EGFR mutation results in overexpression of EGFR (e.g., gene amplification or an increase in EGFR gene copy number). In some embodiments, the EGFR mutation comprises a point mutation or deletion in exon 18, 19, 20, or 21 of the EGFR gene. Known EGFR mutations include, without limitation, an exon 19 deletion, exon 20 insertion, L858R, T790M, S768I, G719A, G719C, G719S, L861Q, C797S, exon 19 insertion, A763_Y764insFQEA, and duplication of the kinase domain. Additional EGFR mutations are described in, e.g., the Atlas of Genetics and Cytogenetics in Oncology and Haematology (see atlasgeneticsoncology.org/Genes/GC_EGFR.html) and OMIM gene ID:131550. Exemplary assays for detecting EGFR mutations include, for example, direct sequencing, denaturing high-performance liquid chromatography (dHPLC), high-resolution melting analysis (HRMA), pyrosequencing, polymerase chain reaction (PCR) to detect specific mutations of interest or to target specific regions of interest, fragment length analysis, cationic conjugated polymer (CCP)-based fluorescence resonance energy transfer (FRET), SmartAMP, peptide nucleic acid (PNA)-mediated PCR clamping, IHC, ARMS, real-time PCR, and next-generation sequencing. See, e.g., Ellison, G. et al. (2013) J. Clin. Pathol. 66:79-89.

Various ALK mutations are known in the art. The ALK gene encodes the anaplastic lymphoma kinase (ALK) receptor tyrosine kinase, also known as CD246 and NBLST3. In some embodiments, the ALK mutation comprises a rearrangement or translocation in the ALK gene, e.g., resulting in a fusion gene such as EML4-ALK, KIF5B-ALK, KLC1-ALK, or TFG-ALK. ALK mutations include, but are not limited to, E13; A20 (V10), E20; A20 (V2), E6a/b; A20 (V3a/b), E14; A20 (V4), E2a/b; A20 (V6), E14; A20 (V7), E15; A20 (V4), E18; A20 (V5), KIF5B-ALK, KLC1-ALK, and TFG-ALK. Additional ALK mutations are described in Shackelford, R. E. et al. (2014) Genes Cancer 5:1-14. Exemplary assays for detecting ALK mutations include, for example, PCR, reverse-transcriptase PCR (RT-PCR), microarray or exon array profiling, fluorescence in situ hybridization (FISH) (e.g., using an ALK break-apart or split-signal probe; see Kwak, E. L. et al. (2010) N. Engl. J. Med. 363:1693-1703), IHC, 5′ rapid amplification of cDNA ends (RACE) analysis, and next-generation sequencing. See, e.g., Shackelford, R. E. et al. (2014) Genes Cancer 5:1-14.

In some embodiments, the cancer is breast cancer. In some embodiments, the cancer is triple-negative breast cancer (TNBC). In some embodiments, the cancer is TNBC (e.g., unresectable locally advanced or metastatic TNBC), and the methods further comprise administering a taxane (e.g., paclitaxel or protein-bound paclitaxel) in combination with the anti-PD-L1 antibody (e.g., atezolizumab). In some embodiments, the cancer is TNBC, and the cancer expresses PD-L1 (e.g., a sample obtained from the cancer shows PD-L1-expressing tumor-infiltrating immune cells covering 1% or more of the tumor area, which can be determined, e.g., using an immunohistochemical assay). In some embodiments, the cancer is TNBC, the cancer expresses PD-L1 (e.g., a sample obtained from the cancer shows PD-L1-expressing tumor-infiltrating immune cells covering 1% or more of the tumor area, which can be determined, e.g., using an immunohistochemical assay), and the methods further comprise administering a taxane (e.g., paclitaxel or protein-bound paclitaxel) in combination with the anti-PD-L1 antibody (e.g., atezolizumab).

In some embodiments, the cancer is small cell lung cancer (SCLC). In some embodiments, the cancer is extensive-stage SCLC (ES-SCLC). In some embodiments, the cancer is extensive-stage SCLC (ES-SCLC), and the methods further comprise administering a platinum-containing chemotherapy (e.g., carboplatin) and a topoisomerase II inhibitor (e.g., etoposide) in combination with the anti-PD-L1 antibody (e.g., atezolizumab).

In some embodiments, including but not limited to treatment of NSCLC, the methods comprise administering to the individual a taxane (e.g., paclitaxel or protein-bound paclitaxel), a platinum-containing chemotherapy (e.g., carboplatin), and optionally an anti-VEGF antibody (e.g., bevacizumab) for 4-6 cycles, then administering to the individual the anti-PD-L1 antibody (e.g., atezolizumab) in two or more 4-week cycles at a dose of 1680 mg.

In some embodiments, including but not limited to treatment of SCLC, the methods comprise administering to the individual a platinum-containing chemotherapy (e.g., carboplatin) and a topoisomerase II inhibitor (e.g., etoposide) for 4 cycles, then administering to the individual the anti-PD-L1 antibody (e.g., atezolizumab) in two or more 4-week cycles at a dose of 1680 mg.

In some embodiments, provided herein are methods for treating a human patient having cancer, wherein the cancer is extensive-stage small cell lung cancer. In some embodiments, the method comprises administering an anti-PD-L1 antibody in combination with carboplatin and etoposide. In some embodiments, the method is a first-line treatment.

In some embodiments, the human patient has been previously untreated, e.g., previously untreated with a chemotherapeutic agent. In some embodiments, the human patient has urothelial carcinoma and has been previously untreated for urothelial carcinoma, e.g., previously untreated with a chemotherapeutic agent. In some embodiments, the cancer is a previously untreated cancer, e.g., previously untreated with a chemotherapeutic agent. In some embodiments, the cancer is a treatment-naïve locally advance or metastatic urothelial carcinoma. In some embodiments, the human patient is cisplatin-ineligible. In some embodiments, the human patient is cisplatin-ineligible, and the cancer is a treatment-naïve locally advance or metastatic urothelial carcinoma.

### Exemplary Methods of Treatment

In some embodiments, the method comprises administering to the human patient an anti-PD-L1 antibody in two or more 4-week or 28-day cycles at a dose of 1680 mg, wherein the anti-PD-L1 antibody is administered to the human patient at a dose of 1680 mg per cycle in each of the two or more 4-week or 28-day cycles (e.g., the anti-PD-L1 antibody is administered once every 4 weeks or every 28 days to the human patient).

In some embodiments, the method comprises administering to the human patient an anti-PD-L1 antibody in two or more 2-week or 14-day cycles at a dose of 840 mg, wherein the anti-PD-L1 antibody is administered to the human patient at a dose of 840 mg per cycle in each of the two or more 2-week or 14-day cycles (e.g., the anti-PD-L1 antibody is administered once every 2 weeks or every 14 days to the human patient).

In some embodiments of the methods described herein, the human patient has an urothelial carcinoma. In some embodiments of the methods described herein, the human patient is an adult human patient with locally advanced or metastatic urothelial carcinoma, wherein the adult human patient is not eligible for cisplatin-containing chemotherapy and whose tumors express PD-L1 (PD-L1 stained tumor-infiltrating immune cells [IC]covering >5% of the tumor area), as determined by a US FDA-approved test. In some embodiments of the methods described herein, the human patient is an adult human patient with locally advanced or metastatic urothelial carcinoma, wherein the adult human patient is not eligible for any platinum-containing chemotherapy regardless of PD-L1 status. In some embodiments of the methods described herein, the human patient is an adult human patient with locally advanced or metastatic urothelial carcinoma, wherein the adult human patient has disease progression during or following any platinum-containing chemotherapy, or within 12 months of neoadjuvant or adjuvant chemotherapy.

In some embodiments of the methods described herein, the human patient has an urothelial carcinoma, wherein the method comprises administering to the human patient an anti-PD-L1 antibody at a dose of 840 mg every 2 weeks. In some embodiments of the methods described herein, the human patient has an urothelial carcinoma, wherein the method comprises administering to the human patient an anti-PD-L1 antibody at a dose of 840 mg every 2 weeks, and wherein the anti-PD-L1 antibody is administered intravenously over 60 minutes until disease progression or unacceptable toxicity. In some embodiments of the methods described herein, the human patient has an urothelial carcinoma, wherein the method comprises administering to the human patient an anti-PD-L1 antibody at a dose of 840 mg every 2 weeks, wherein the anti-PD-L1 antibody is administered intravenously over 60 minutes until disease progression or unacceptable toxicity, and wherein, if the first infusion of the anti-PD-L1 antibody is tolerated, all subsequent infusions may be delivered over 30 minutes.

In some embodiments of the methods described herein, the human patient has an urothelial carcinoma, wherein the method comprises administering to the human patient an anti-PD-L1 antibody at a dose of 1680 mg every 4 weeks. In some embodiments of the methods described herein, the human patient has an urothelial carcinoma, wherein the method comprises administering to the human patient an anti-PD-L1 antibody at a dose of 1680 mg every 4 weeks, and wherein the anti-PD-L1 antibody is administered intravenously over 60 minutes until disease progression or unacceptable toxicity. In some embodiments of the methods described herein, the human patient has an urothelial carcinoma, wherein the method comprises administering to the human patient an anti-PD-L1 antibody at a dose of 1680 mg every 4 weeks, wherein the anti-PD-L1 antibody is administered intravenously over 60 minutes until disease progression or unacceptable toxicity, and wherein, if the first infusion of the anti-PD-L1 antibody is tolerated, all subsequent infusions may be delivered over 30 minutes.

In some embodiments of the methods described herein, the human patient has non-small cell lung cancer (NSCLC). In some embodiments of the methods described herein, the human patient is an adult human patient, wherein the adult human patient has metastatic non-squamous NSCLC. In some embodiments of the methods described herein, the adult human patient has metastatic non-squamous NSCLC, wherein the method comprises administering to the adult human patient an anti-PD-L1 antibody in combination with bevacizumab, paclitaxel, and carboplatin. In some embodiments of the methods described herein, the method is a first-line treatment of an adult human patient with metastatic non-squamous NSCLC with no EGFR or ALK genomic tumor aberrations.

In some embodiments of the methods described herein, the human patient is an adult human patient, wherein the adult human patient has metastatic NSCLC, wherein the adult human patient has disease progression during or following a platinum-containing chemotherapy. In some embodiments of the methods described herein, the human patient has NSCLC, wherein the human patient has an EGFR or ALK genomic tumor aberration, and wherein the human patient had disease progression on FDA-approved therapy for NSCLC harboring these aberrations prior to being administered an anti-PD-L1 antibody according to a method described herein. In some embodiments of the methods described herein, the method comprising administering an anti-PD-L1 antibody is single-agent treatment.

In some embodiments of the methods described herein, the human patient is an adult human patient, wherein the adult human patient has metastatic non-squamous NSCLC with no EGFR or ALK genomic tumor aberrations, and wherein the method comprises administering an anti-PD-L1 antibody in combination with bevacizumab, paclitaxel, and carboplatin. In some embodiments of the methods described herein, the method is indicated for the first-line treatment of adult patients with metastatic non-squamous NSCLC with no EGFR or ALK genomic tumor aberrations.

In some embodiments of the methods described herein, the human patient has a NSCLC, wherein an anti-PD-L1 antibody is administered until disease progression or unacceptable toxicity.

In some embodiments of the methods described herein, the human patient has a NSCLC, wherein an anti-PD-L1 antibody is administered prior to chemotherapy or other antineoplastic drugs when administered to the human patient on the same day.

In some embodiments of the methods described herein, the human patient has NSCLC, wherein the method comprises administering an anti-PD-L1 antibody as a single agent at a dose of 840 mg every 2 weeks, 1200 mg every 3 weeks, or 1680 mg every 4 weeks.

In some embodiments of the methods described herein, the human patient has a NSCLC, wherein the method comprises administering to the human patient an anti-PD-L1 antibody at a dose of 840 mg every 2 weeks. In some embodiments of the methods described herein, the human patient has a NSCLC, wherein the method comprises administering to the human patient an anti-PD-L1 antibody at a dose of 840 mg every 2 weeks, and wherein the anti-PD-L1 antibody is administered intravenously over 60 minutes until disease progression or unacceptable toxicity. In some embodiments of the methods described herein, the human patient has a NSCLC, wherein the method comprises administering to the human patient an anti-PD-L1 antibody at a dose of 840 mg every 2 weeks, wherein the anti-PD-L1 antibody is administered intravenously over 60 minutes until disease progression or unacceptable toxicity, and wherein, if the first infusion of the anti-PD-L1 antibody is tolerated, all subsequent infusions may be delivered over 30 minutes. In some embodiments of the methods described herein, the anti-PD-L1 antibody is administered in combination with bevacizumab at a dose of the standard of care, paclitaxel at a dose of the standard of care, and carboplatin at a dose of the standard of care, until disease progression or unacceptable toxicity. In some embodiments of the methods described herein, the anti-PD-L1 antibody is administered in combination with bevacizumab at a dose of 15 mg/kg, paclitaxel at a dose of 175 mg/m² or 200 mg/m², and carboplatin at a dose of AUC 6 mg/mL/min, until disease progression or unacceptable toxicity. In some embodiments of the methods described herein, wherein the anti-PD-L1 antibody is administered in combination with bevacizumab, paclitaxel, and carboplatin, the anti-PD-L1 antibody is administered prior to other antineoplastic drugs when given on the same day. In some embodiments of the methods described herein, following completion of 4-6 cycles of a method comprising administering to a human patient an anti-PD-L1 antibody in combination with bevacizumab, paclitaxel, and carboplatin, if bevacizumab is discontinued, the method comprises further administering the anti-PD-L1 antibody at a dose of 840 mg every 2 weeks, administered intravenously until disease progression or unacceptable toxicity. In some embodiments of the methods described herein, following completion of 4-6 cycles of a method comprising administering to a human patient an anti-PD-L1 antibody in combination with bevacizumab, paclitaxel, and carboplatin, if bevacizumab is discontinued, the method comprises further administering the anti-PD-L1 antibody at a dose of 1680 mg every 4 weeks, administered intravenously until disease progression or unacceptable toxicity. In some embodiments of the methods described herein, the initial infusion of an anti-PD-L1 antibody over 60 minutes. In some embodiments of the methods described herein, if the initial infusion of an anti-PD-L1 antibody is tolerated, all subsequent infusions are delivered over 30 minutes.

In some embodiments of the methods described herein, the human patient has a NSCLC, wherein the method comprises administering to the human patient an anti-PD-L1 antibody at a dose of 1680 mg every 4 weeks. In some embodiments of the methods described herein, the human patient has a NSCLC, wherein the method comprises administering to the human patient an anti-PD-L1 antibody at a dose of 1680 mg every 4 weeks, and wherein the anti-PD-L1 antibody is administered intravenously over 60 minutes until disease progression or unacceptable toxicity. In some embodiments of the methods described herein, the human patient has a NSCLC, wherein the method comprises administering to the human patient an anti-PD-L1 antibody at a dose of 1680 mg every 4 weeks, wherein the anti-PD-L1 antibody is administered intravenously over 60 minutes until disease progression or unacceptable toxicity, and wherein, if the first infusion of the anti-PD-L1 antibody is tolerated, all subsequent infusions may be delivered over 30 minutes. In some embodiments of the methods described herein, the anti-PD-L1 antibody is administered in combination with bevacizumab at a dose of the standard of care, paclitaxel at a dose of the standard of care, and carboplatin at a dose of the standard of care, until disease progression or unacceptable toxicity. In some embodiments of the methods described herein, the anti-PD-L1 antibody is administered in combination with bevacizumab at a dose of 15 mg/kg, paclitaxel at a dose of 175 mg/m² or 200 mg/m², and carboplatin at a dose of AUC 6 mg/mL/min, until disease progression or unacceptable toxicity. In some embodiments of the methods described herein, wherein the anti-PD-L1 antibody is administered in combination with bevacizumab, paclitaxel, and carboplatin, the anti-PD-L1 antibody is administered prior to other antineoplastic drugs when given on the same day. In some embodiments of the methods described herein, following completion of 4-6 cycles of a method comprising administering to a human patient an anti-PD-L1 antibody in combination with bevacizumab, paclitaxel, and carboplatin, if bevacizumab is discontinued, the method comprises further administering the anti-PD-L1 antibody at a dose of 840 mg every 2 weeks, administered intravenously until disease progression or unacceptable toxicity. In some embodiments of the methods described herein, following completion of 4-6 cycles of a method comprising administering to a human patient an anti-PD-L1 antibody in combination with bevacizumab, paclitaxel, and carboplatin, if bevacizumab is discontinued, the method comprises further administering the anti-PD-L1 antibody at a dose of 1680 mg every 4 weeks, administered intravenously until disease progression or unacceptable toxicity. In some embodiments of the methods described herein, the initial infusion of an anti-PD-L1 antibody over 60 minutes. In some embodiments of the methods described herein, if the initial infusion of an anti-PD-L1 antibody is tolerated, all subsequent infusions are delivered over 30 minutes.

In some embodiments of the methods described herein, the human patient has a NSCLC, wherein an anti-PD-L1 antibody is administered in combination with bevacizumab, paclitaxel, and carboplatin, the anti-PD-L1 antibody is administered at a dose of 1200 mg every 3 weeks prior to chemotherapy or other antineoplastic drugs.

In some embodiments of the methods described herein, the human patient has a NSCLC, wherein following completion of 4-6 cycles of paclitaxel and carboplatin, and if bevacizumab is discontinued, an anti-PD-L1 antibody is administered at a dose of 840 mg every 2 weeks, 1200 mg every 3 weeks, or 1680 mg every 4 weeks.

In some embodiments of the methods described herein, the human patient is an adult human patient, wherein the adult human patient has triple-negative breast cancer (TNBC). In some embodiments of the methods described herein, the human patient is an adult human patient, wherein the adult human patient has unresectable locally advanced or metastatic TNBC, wherein a tumour of the unresectable locally advanced or metastatic TNBC expresses PD-L1 (PD-L1 stained tumor-infiltrating immune cells [IC] of any intensity covering >1% of the tumor area), as determined by a US FDA-approved test.

In some embodiments of the methods described herein, the adult human patient has metastatic TNBC, wherein the method comprises administering an anti-PD-L1 antibody at a dose of 840 mg followed by paclitaxel protein-bound at a dose of 100 mg/m², wherein for each 28-day cycle, the anti-PD-L1 antibody is administered on days 1 and 15, and paclitaxel protein-bound is administered on days 1, 8, and 15, until disease progression or unacceptable toxicity. In some embodiments of the methods described herein, the adult human patient has locally advanced or metastatic TNBC, wherein the method comprises administering an anti-PD-L1 antibody at a dose of 840 mg and paclitaxel protein-bound at a dose of 100 mg/m², wherein the anti-PD-L1 antibody is administered as an intravenous infusion, over 60 minutes, followed by administration of 100 mg/m² paclitaxel protein-bound, wherein for each 28-day cycle, the anti-PD-L1 antibody is administered on days 1 and 15, and paclitaxel protein-bound is administered on days 1, 8, and 15, until disease progression or unacceptable toxicity. In some embodiments of the methods described herein, the initial infusion of an anti-PD-L1 antibody is infused over 60 minutes. In some embodiments of the methods described herein, if the initial infusion of an anti-PD-L1 antibody over 60 minutes is tolerated, all subsequent infusions may be delivered over 30 minutes.

In some embodiments of the methods described herein, the human patient is an adult human patient, wherein the adult human patient has extensive-stage small cell lung cancer (ES-SCLC). In some embodiments of the methods described herein, the adult human patient has ES-SCLC, and wherein the adult human patient is indicated for the first-line treatment using a method described herein comprising an anti-PD-L1 antibody in combination with carboplatin and etoposide.

In some embodiments of the methods described herein, the human patient has SCLC, wherein following completion of 4 cycles of carboplatin and etoposide, the method comprises administering to the human patient a treatment comprising an anti-PD-L1 antibody administered at a dose of 840 mg every 2 weeks, 1200 mg every 3 weeks, or 1680 mg every 4 weeks. In some embodiments of the methods described herein, the human patient has SCLC, wherein the human patient has received 4 cycles of an initial treatment comprising carboplatin and etoposide, wherein following completion of 4 cycles of the initial treatment, the method comprises administering to the human patient a treatment comprising an anti-PD-L1 antibody administered at a dose of 840 mg every 2 weeks administered intravenously until disease progression or unacceptable toxicity. In some embodiments of the methods described herein, the human patient has SCLC, wherein the human patient has received 4 cycles of an initial treatment comprising carboplatin and etoposide, wherein following completion of 4 cycles of the initial treatment, the method comprises administering to the human patient a treatment comprising an anti-PD-L1 antibody administered at a dose of 1680 mg every 4 weeks administered intravenously until disease progression or unacceptable toxicity. In some embodiments, the initial treatment further comprises administering an anti-PD-L1 antibody at a dose of 1200 mg every 3 weeks. In some embodiments of the methods described herein, the initial infusion of an anti-PD-L1 antibody is infused over 60 minutes. In some embodiments of the methods described herein, if the initial infusion of an anti-PD-L1 antibody over 60 minutes is tolerated, all subsequent infusions may be delivered over 30 minutes.

In some embodiments of the methods described herein, the human patient has SCLC, wherein when administering an anti-PD-L1 antibody with carboplatin and etoposide, the anti-PD-L1 antibody is administered at a dose of 1200 mg every 3 weeks prior to chemotherapy.

In some embodiments of the methods described herein, the human patient has a SCLC, wherein an anti-PD-L1 antibody is administered prior to chemotherapy when administered to the human patient on the same day.

### III. Anti-PD-L1 Antibodies

A variety of anti-PDL1 antibodies are contemplated for use in the methods of the present disclosure and described herein. In any of the embodiments herein, the isolated anti-PDL1 antibody can bind to a human PDL1, for example a human PDL1 as shown in UniProtKB/Swiss-Prot Accession No. Q9NZQ7.1, or a variant thereof. Alternative names for “PDL1” include B7-H1, B7-4, CD274, and B7-H.

In some embodiments, the anti-PDL1 antibody is capable of inhibiting binding between PDL1 and PD-1 and/or between PDL1 and B7-1. In some embodiments, the anti-PDL1 antibody is a monoclonal antibody. In some embodiments, the anti-PDL1 antibody is an antibody fragment selected from the group consisting of Fab, Fab′-SH, Fv, scFv, and (Fab′)₂ fragments. In some embodiments, the anti-PDL1 antibody is a humanized antibody. In some embodiments, the anti-PDL1 antibody is a human antibody. Examples of anti-PDL1 antibodies useful for the methods of this invention, and methods for making thereof are described in PCT patent application WO 2010/077634 A1 and U.S. Pat. No. 8,217,149, which are incorporated herein by reference.

In some embodiments, the anti-PDL1 antibody comprises a heavy chain variable region and a light chain variable region, wherein:
(a) the heavy chain variable region comprises an HVR-H1, HVR-H2, and HVR-H3 sequence of GFTFSDSWIH (SEQ ID NO:1), AWISPYGGSTYYADSVKG (SEQ ID NO:2) and RHWPGGFDY (SEQ ID NO:3), respectively, and
(b) the light chain variable region comprises an HVR-L1, HVR-L2, and HVR-L3 sequence of RASQDVSTAVA (SEQ ID NO:4), SASFLYS (SEQ ID NO:5) and QQYLYHPAT (SEQ ID NO:6), respectively.

In some embodiments, the anti-PDL1 antibody is MPDL3280A, also known as atezolizumab and TECENTRIQ® (CAS Registry Number: 1422185-06-5). In some embodiments, the anti-PDL1 antibody comprises a heavy chain and a light chain sequence, wherein:
(a) the heavy chain variable region sequence comprises the amino acid sequence:

(SEQ ID NO: 7)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSS,

and
(b) the light chain variable region sequence comprises the amino acid sequence:

(SEQ ID NO: 8)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In some embodiments, the anti-PDL1 antibody comprises a heavy chain and a light chain sequence, wherein:
(a) the heavy chain comprises the amino acid sequence:

(SEQ ID NO: 9)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPG,

and
(b) the light chain comprises the amino acid sequence:

(SEQ ID NO: 10)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some embodiments, the anti-PDL1 antibody is avelumab (CAS Registry Number: 1537032-82-8). Avelumab, also known as MSB0010718C, is a human monoclonal IgG1 anti-PDL1 antibody (Merck KGaA, Pfizer). In some embodiments, the anti-PDL1 antibody comprises a heavy chain and a light chain sequence, wherein:
(a) the heavy chain comprises the amino acid sequence:

(SEQ ID NO: 15)
EVQLLESGGGLVQPGGSLRLSCAASGFTFSSYIMMWVRQAPGKGLEWVS
SIYPSGGITFYADTVKGRFTISRDNSKNTLYLQMNSLRAEDTAVYYCAR
IKLGTVTTVDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPG,

and
(b) the light chain comprises the amino acid sequence:

(SEQ ID NO: 16)
QSALTQPASVSGSPGQSITISCTGTSSDVGGYNYVSWYQQHPGKAPKLM
IYDVSNRPSGVSNRFSGSKSGNTASLTISGLQAEDEADYYCSSYTSSST
RVFGTGTKVTVLGQPKANPTVTLFPPSSEELQANKATLVCLISDFYPGA
VTVAWKADGSPVKAGVETTKPSKQSNNKYAASSYLSLTPEQWKSHRSYS
CQVTHEGSTVEKTVAPTECS.

In some embodiments, the anti-PDL1 antibody comprises the six HVR sequences from SEQ ID NO:15 and SEQ ID NO:16 (e.g., the three heavy chain HVRs from SEQ ID NO:15 and the three light chain HVRs from SEQ ID NO:16). In some embodiments, the anti-PDL1 antibody comprises the heavy chain variable domain from SEQ ID NO:15 and the light chain variable domain from SEQ ID NO:16.

In some embodiments, the anti-PDL1 antibody is durvalumab (CAS Registry Number: 1428935-60-7). Durvalumab, also known as MEDI4736, is an Fc optimized human monoclonal IgG1 kappa anti-PDL1 antibody (MedImmune, AstraZeneca) described in WO2011/066389 and US2013/034559. In some embodiments, the anti-PDL1 antibody comprises a heavy chain and a light chain sequence, wherein:
(a) the heavy chain comprises the amino acid sequence:

(SEQ ID NO: 17)
EVQLVESGGGLVQPGGSLRLSCAASGFTFSRYWMSWVRQAPGKGLEWVA
NIKQDGSEKYYVDSVKGRFTISRDNAKNSLYLQMNSLRAEDTAVYYCAR
EGGWFGELAFDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKRVEPKSCDKTHTCPPCPAPEFEGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPASIEKTISKA
KGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYT
QKSLSLSPG,

and
(b) the light chain comprises the amino acid sequence:

(SEQ ID NO: 18)
EIVLTQSPGTLSLSPGERATLSCRASQRVSSSYLAWYQQKPGQAPRLLI
YDASSRATGIPDRFSGSGSGTDFTLTISRLEPEDFAVYYCQQYGSLPWT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

In some embodiments, the anti-PDL1 antibody comprises the six HVR sequences from SEQ ID NO:17 and SEQ ID NO:18 (e.g., the three heavy chain HVRs from SEQ ID NO:17 and the three light chain HVRs from SEQ ID NO:18). In some embodiments, the anti-PDL1 antibody comprises the heavy chain variable domain from SEQ ID NO:17 and the light chain variable domain from SEQ ID NO:18.

In some embodiments, the anti-PDL1 antibody is MDX-1105 (Bristol Myers Squibb). MDX-1105, also known as BMS-936559, is an anti-PDL1 antibody described in WO2007/005874.

In some embodiments, the anti-PDL1 antibody is LY3300054 (Eli Lilly).

In some embodiments, the anti-PDL1 antibody is STI-A1014 (Sorrento). STI-A1014 is a human anti-PDL1 antibody.

In some embodiments, the anti-PDL1 antibody is KN035 (Suzhou Alphamab). KN035 is single-domain antibody (dAB) generated from a camel phage display library.

In some embodiments, the anti-PDL1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some embodiments, the anti-PDL1 antibody is CX-072 (CytomX Therapeutics).

In some embodiments, the PDL1 antibody comprises the six HVR sequences (e.g., the three heavy chain HVRs and the three light chain HVRs) and/or the heavy chain variable domain and light chain variable domain from a PDL1 antibody described in US20160108123 (Assigned to Novartis), WO2016/000619 (Applicant: Beigene), WO2012/145493 (Applicant: Amplimmune), U.S. Pat. No. 9,205,148 (Assigned to MedImmune), WO2013/181634 (Applicant: Sorrento), and WO2016/061142 (Applicant: Novartis).

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A.

In a still further specific aspect, the antibody has reduced or minimal effector function. In a still further specific aspect the minimal effector function results from an “effector-less Fc mutation” or aglycosylation mutation. In still a further embodiment, the effector-less Fc mutation is an N297A or D265A/N297A substitution in the constant region. In some embodiments, the isolated anti-PDL1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In a still further embodiment, the present disclosure provides for compositions comprising any of the above described anti-PDL1 antibodies in combination with at least one pharmaceutically-acceptable carrier. Any of the pharmaceutically acceptable carriers described herein or known in the art may be used.

### IV. Antibody Preparation

The antibodies described herein are prepared using techniques available in the art for generating antibodies, exemplary methods of which are described in more detail in the following sections.

The antibody is directed against an antigen of interest (e.g., PD-L1, such as a human PD-L1). Preferably, the antigen is a biologically important polypeptide and administration of the antibody to a mammal suffering from a disorder can result in a therapeutic benefit in that mammal.

In certain embodiments, an antibody provided herein has a dissociation constant (Kd) of ≤1 M, ≤150 nM, ≤100 nM, ≤50 nM, ≤10 nM, ≤1 nM, ≤0.1 nM, ≤0.01 nM, or ≤0.001 nM (e.g. 10-8 M or less, e.g. from 10-8 M to 10-13 M, e.g., from 10-9 M to 10-13 M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [125I]-antigen are mixed with serial dilutions of a Fab of interest. The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, NJ) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 g/ml (˜0.2 M) before injection at a flow rate of 5/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 l/min. Association rates (kon) and dissociation rates (koff) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### Chimeric, Humanized and Human Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### Antibody Fragments

Antibody fragments may be generated by traditional means, such as enzymatic digestion, or by recombinant techniques. In certain circumstances there are advantages of using antibody fragments, rather than whole antibodies. The smaller size of the fragments allows for rapid clearance, and may lead to improved access to solid tumors. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-167 (1992)). According to another approach, F(ab′) 2 fragments can be isolated directly from recombinant host cell culture. Fab and F(ab′) 2 fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific.

### Single-Domain Antibodies

In some embodiments, an antibody of the present disclosure is a single-domain antibody. A single-domain antibody is a single polypeptide chain comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1). In one embodiment, a single-domain antibody consists of all or a portion of the heavy chain variable domain of an antibody.

### Antibody Variants

In some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

### Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table A. More substantial changes are further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE A
Conservative Substitutions.

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
a. hydrophobic: Norleucine, Met, Ala, Val, Len, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the present disclosure may be made in order to create antibody variants with certain improved properties.

In one embodiment, antibody variants are provided comprising an Fc region wherein a carbohydrate structure attached to the Fc region has reduced fucose or lacks fucose, which may improve ADCC function. Specifically, antibodies are contemplated herein that have reduced fucose relative to the amount of fucose on the same antibody produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In certain embodiments, the antibody is one wherein less than about 50%, 40%, 30%, 20%, 10%, or 5% of the N-linked glycans thereon comprise fucose. For example, the amount of fucose in such an antibody may be from 1% to 80%, from 1% to 65%, from 5% to 65% or from 20% to 40%. In certain embodiments, the antibody is one wherein none of the N-linked glycans thereon comprise fucose, i.e., wherein the antibody is completely without fucose, or has no fucose or is afucosylated. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, the antibody variants comprising an Fc region described herein are capable of binding to an FcγRIII. In certain embodiments, the antibody variants comprising an Fc region described herein have ADCC activity in the presence of human effector cells or have increased ADCC activity in the presence of human effector cells compared to the otherwise same antibody comprising a human wild-type IgG1Fc region.

### Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (Cell Technology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues). In an exemplary embodiment, the antibody comprising the following amino acid substitutions in its Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.)). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

### V. Pharmaceutical Compositions and Formulations

Also provided herein are pharmaceutical compositions and formulations, e.g., for the treatment of cancer, comprising an anti-PD-L1 antibody (e.g., atezolizumab). In some embodiments, the pharmaceutical compositions and formulations further comprise a pharmaceutically acceptable carrier.

In some embodiments, an anti-PDL1 antibody described herein (such as atezolizumab) is in a formulation comprising the antibody at an amount of about 60 mg/mL, histidine acetate in a concentration of about 20 mM, sucrose in a concentration of about 120 mM, and polysorbate (e.g., polysorbate 20) in a concentration of 0.04% (w/v), and the formulation has a pH of about 5.8. In some embodiments, the anti-PDL1 antibody described herein (such as atezolizumab) is in a formulation comprising the antibody in an amount of about 125 mg/mL, histidine acetate in a concentration of about 20 mM, sucrose is in a concentration of about 240 mM, and polysorbate (e.g., polysorbate 20) in a concentration of 0.02% (w/v), and the formulation has a pH of about 5.5.

After preparation of the antibody of interest (e.g., techniques for producing antibodies which can be formulated as disclosed herein are elaborated herein and are known in the art), the pharmaceutical formulation comprising it is prepared. In certain embodiments, the antibody to be formulated has not been subjected to prior lyophilization and the formulation of interest herein is an aqueous formulation. In certain embodiments, the antibody is a full length antibody. In one embodiment, the antibody in the formulation is an antibody fragment, such as an F(ab′)2, in which case problems that may not occur for the full length antibody (such as clipping of the antibody to Fab) may need to be addressed. The therapeutically effective amount of antibody present in the formulation is determined by taking into account the desired dose volumes and mode(s) of administration, for example. From about 25 mg/mL to about 150 mg/mL, or from about 30 mg/mL to about 140 mg/mL, or from about 35 mg/mL to about 130 mg/mL, or from about 40 mg/mL to about 120 mg/mL, or from about 50 mg/mL to about 130 mg/mL, or from about 50 mg/mL to about 125 mg/mL, or from about 50 mg/mL to about 120 mg/mL, or from about 50 mg/mL to about 110 mg/mL, or from about 50 mg/mL to about 100 mg/mL, or from about 50 mg/mL to about 90 mg/mL, or from about 50 mg/mL to about 80 mg/mL, or from about 54 mg/mL to about 66 mg/mL is an exemplary antibody concentration in the formulation. In some embodiments, an anti-PDL1 antibody described herein (such as atezolizumab) is administered at a dose of about 1200 mg.

An aqueous formulation is prepared comprising the antibody in a pH-buffered solution. In some embodiments, the buffer of the present disclosure has a pH in the range from about 5.0 to about 7.0. In certain embodiments the pH is in the range from about 5.0 to about 6.5, the pH is in the range from about 5.0 to about 6.4, in the range from about 5.0 to about 6.3, the pH is in the range from about 5.0 to about 6.2, the pH is in the range from about 5.0 to about 6.1, the pH is in the range from about 5.5 to about 6.1, the pH is in the range from about 5.0 to about 6.0, the pH is in the range from about 5.0 to about 5.9, the pH is in the range from about 5.0 to about 5.8, the pH is in the range from about 5.1 to about 6.0, the pH is in the range from about 5.2 to about 6.0, the pH is in the range from about 5.3 to about 6.0, the pH is in the range from about 5.4 to about 6.0, the pH is in the range from about 5.5 to about 6.0, the pH is in the range from about 5.6 to about 6.0, the pH is in the range from about 5.7 to about 6.0, or the pH is in the range from about 5.8 to about 6.0. In some embodiments, the formulation has a pH of 6.0 or about 6.0. In some embodiments, the formulation has a pH of 5.9 or about 5.9. In some embodiments, the formulation has a pH of 5.8 or about 5.8. In some embodiments, the formulation has a pH of 5.7 or about 5.7. In some embodiments, the formulation has a pH of 5.6 or about 5.6. In some embodiments, the formulation has a pH of 5.5 or about 5.5. In some embodiments, the formulation has a pH of 5.4 or about 5.4. In some embodiments, the formulation has a pH of 5.3 or about 5.3. In some embodiments, the formulation has a pH of 5.2 or about 5.2. Examples of buffers that will control the pH within this range include histidine (such as L-histidine) or sodium acetate. In certain embodiments, the buffer contains histidine acetate or sodium acetate in the concentration of about 15 mM to about 25 mM. In some embodiments, the buffer contains histidine acetate or sodium acetate in the concentration of about 15 mM to about 25 mM, about 16 mM to about 25 mM, about 17 mM to about 25 mM, about 18 mM to about 25 mM, about 19 mM to about 25 mM, about 20 mM to about 25 mM, about 21 mM to about 25 mM, about 22 mM to about 25 mM, about 15 mM, about 16 mM, about 17 mM, about 18 mM, about 19 mM, about 20 mM, about 21 mM, about 22 mM, about 23 mM, about 24 mM, or about 25 mM. In one embodiment, the buffer is histidine acetate or sodium acetate in an amount of about 20 mM, pH 5.0. In one embodiment, the buffer is histidine acetate or sodium acetate in an amount of about 20 mM, pH 5.1. In one embodiment, the buffer is histidine acetate or sodium acetate in an amount of about 20 mM, pH 5.2. In one embodiment, the buffer is histidine acetate or sodium acetate in an amount of about 20 mM, pH 5.3. In one embodiment, the buffer is histidine acetate or sodium acetate in an amount of about 20 mM, pH 5.4. In one embodiment, the buffer is histidine acetate or sodium acetate in an amount of about 20 mM, pH 5.5. In one embodiment, the buffer is histidine acetate or sodium acetate in an amount of about 20 mM, pH 5.6. In one embodiment, the buffer is histidine acetate or sodium acetate in an amount of about 20 mM, pH 5.7. In one embodiment, the buffer is histidine acetate or sodium acetate in an amount of about 20 mM, pH 5.8. In one embodiment, the buffer is histidine acetate or sodium acetate in an amount of about 20 mM, pH 5.9. In one embodiment, the buffer is histidine acetate or sodium acetate in an amount of about 20 mM, pH 6.0. In one embodiment, the buffer is histidine acetate or sodium acetate in an amount of about 20 mM, pH 6.1. In one embodiment, the buffer is histidine acetate or sodium acetate in an amount of about 20 mM, pH 6.2. In one embodiment, the buffer is histidine acetate or sodium acetate in an amount of about 20 mM, pH 6.3. In one embodiment, the buffer is histidine acetate or sodium acetate in an amount of about 25 mM, pH 5.2. In one embodiment, the buffer is histidine acetate or sodium acetate in an amount of about 25 mM, pH 5.3. In one embodiment, the buffer is histidine acetate or sodium acetate in an amount of about 25 mM, pH 5.4. In one embodiment, the buffer is histidine acetate or sodium acetate in an amount of about 25 mM, pH 5.5. In one embodiment, the buffer is histidine acetate or sodium acetate in an amount of about 25 mM, pH 5.6. In one embodiment, the buffer is histidine acetate or sodium acetate in an amount of about 25 mM, pH 5.7. In one embodiment, the buffer is histidine acetate or sodium acetate in an amount of about 25 mM, pH 5.8. In one embodiment, the buffer is histidine acetate or sodium acetate in an amount of about 25 mM, pH 5.9. In one embodiment, the buffer is histidine acetate or sodium acetate in an amount of about 25 mM, pH 6.0. In one embodiment, the buffer is histidine acetate or sodium acetate in an amount of about 25 mM, pH 6.1. In one embodiment, the buffer is histidine acetate or sodium acetate in an amount of about 25 mM, pH 6.2. In one embodiment, the buffer is histidine acetate or sodium acetate in an amount of about 25 mM, pH 6.3.

In some embodiments, the formulation further comprises sucrose in an amount of about 60 mM to about 240 mM. In some embodiments, sucrose in the formulation is about 60 mM to about 230 mM, about 60 mM to about 220 mM, about 60 mM to about 210 mM, about 60 mM to about 200 mM, about 60 mM to about 190 mM, about 60 mM to about 180 mM, about 60 mM to about 170 mM, about 60 mM to about 160 mM, about 60 mM to about 150 mM, about 60 mM to about 140 mM, about 80 mM to about 240 mM, about 90 mM to about 240 mM, about 100 mM to about 240 mM, about 110 mM to about 240 mM, about 120 mM to about 240 mM, about 130 mM to about 240 mM, about 140 mM to about 240 mM, about 150 mM to about 240 mM, about 160 mM to about 240 mM, about 170 mM to about 240 mM, about 180 mM to about 240 mM, about 190 mM to about 240 mM, about 200 mM to about 240 mM, about 80 mM to about 160 mM, about 100 mM to about 140 mM, or about 110 mM to about 130 mM. In some embodiments, sucrose in the formulation is about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, or about 240 mM.

In some embodiments, the antibody concentration in the formulation is about 40 mg/ml to about 125 mg/ml. In some embodiments, the antibody concentration in the formulation is about 40 mg/ml to about 120 mg/ml, about 40 mg/ml to about 110 mg/ml, about 40 mg/ml to about 100 mg/ml, about 40 mg/ml to about 90 mg/ml, about 40 mg/ml to about 80 mg/ml, about 40 mg/ml to about 70 mg/ml, about 50 mg/ml to about 120 mg/ml, about 60 mg/ml to about 120 mg/ml, about 70 mg/ml to about 120 mg/ml, about 80 mg/ml to about 120 mg/ml, about 90 mg/ml to about 120 mg/ml, or about 100 mg/ml to about 120 mg/ml. In some embodiments, the antibody concentration in the formulation is about 60 mg/ml. In some embodiments, the antibody concentration in the formulation is about 65 mg/ml. In some embodiments, the antibody concentration in the formulation is about 70 mg/ml. In some embodiments, the antibody concentration in the formulation is about 75 mg/ml. In some embodiments, the antibody concentration in the formulation is about 80 mg/ml. In some embodiments, the antibody concentration in the formulation is about 85 mg/ml. In some embodiments, the antibody concentration in the formulation is about 90 mg/ml. In some embodiments, the antibody concentration in the formulation is about 95 mg/ml. In some embodiments, the antibody concentration in the formulation is about 100 mg/ml. In some embodiments, the antibody concentration in the formulation is about 110 mg/ml. In some embodiments, the antibody concentration in the formulation is about 125 mg/ml. In some embodiments, an anti-PDL1 antibody described herein (such as atezolizumab) is administered at a concentration of about 60 mg/mL.

In some embodiments, a surfactant is added to the antibody formulation. Exemplary surfactants include nonionic surfactants such as polysorbates (e.g. polysorbates 20, 80 etc) or poloxamers (e.g. poloxamer 188, etc.). The amount of surfactant added is such that it reduces aggregation of the formulated antibody and/or minimizes the formation of particulates in the formulation and/or reduces adsorption. For example, the surfactant may be present in the formulation in an amount from about 0.001% to about 0.5% (w/v). In some embodiments, the surfactant (e.g., polysorbate 20) is from about 0.005% to about 0.2%, from about 0.005% to about 0.1%, from about 0.005% to about 0.09%, from about 0.005% to about 0.08%, from about 0.005% to about 0.07%, from about 0.005% to about 0.06%, from about 0.005% to about 0.05%, from about 0.005% to about 0.04%, from about 0.008% to about 0.06%, from about 0.01% to about 0.06%, from about 0.02% to about 0.06%, from about 0.01% to about 0.05%, or from about 0.02% to about 0.04%. In certain embodiments, the surfactant (e.g., polysorbate 20) is present in the formulation in an amount of 0.005% or about 0.005%. In certain embodiments, the surfactant (e.g., polysorbate 20) is present in the formulation in an amount of 0.006% or about 0.006%. In certain embodiments, the surfactant (e.g., polysorbate 20) is present in the formulation in an amount of 0.007% or about 0.007%. In certain embodiments, the surfactant (e.g., polysorbate 20) is present in the formulation in an amount of 0.008% or about 0.008%. In certain embodiments, the surfactant (e.g., polysorbate 20) is present in the formulation in an amount of 0.009% or about 0.009%. In certain embodiments, the surfactant (e.g., polysorbate 20) is present in the formulation in an amount of 0.01% or about 0.01%. In certain embodiments, the surfactant (e.g., polysorbate 20) is present in the formulation in an amount of 0.02% or about 0.02%. In certain embodiments, the surfactant (e.g., polysorbate 20) is present in the formulation in an amount of 0.03% or about 0.03%. In certain embodiments, the surfactant (e.g., polysorbate 20) is present in the formulation in an amount of 0.04% or about 0.04%. In certain embodiments, the surfactant (e.g., polysorbate 20) is present in the formulation in an amount of 0.05% or about 0.05%. In certain embodiments, the surfactant (e.g., polysorbate 20) is present in the formulation in an amount of 0.06% or about 0.06%. In certain embodiments, the surfactant (e.g., polysorbate 20) is present in the formulation in an amount of 0.07% or about 0.07%. In certain embodiments, the surfactant (e.g., polysorbate 20) is present in the formulation in an amount of 0.08% or about 0.08%. In certain embodiments, the surfactant (e.g., polysorbate 20) is present in the formulation in an amount of 0.1% or about 0.1%. In certain embodiments, the surfactant (e.g., polysorbate 20) is present in the formulation in an amount of 0.2% or about 0.2%. In certain embodiments, the surfactant (e.g., polysorbate 20) is present in the formulation in an amount of 0.3% or about 0.3%. In certain embodiments, the surfactant (e.g., polysorbate 20) is present in the formulation in an amount of 0.4% or about 0.4%. In certain embodiments, the surfactant (e.g., polysorbate 20) is present in the formulation in an amount of 0.5% or about 0.5%.

In one embodiment, the formulation contains the above-identified agents (e.g., antibody, buffer, sucrose, and/or surfactant) and is essentially free of one or more preservatives, such as benzyl alcohol, phenol, m-cresol, chlorobutanol and benzethonium Cl. In another embodiment, a preservative may be included in the formulation, particularly where the formulation is a multidose formulation. The concentration of preservative may be in the range from about 0.1% to about 2%, preferably from about 0.5% to about 1%. One or more other pharmaceutically acceptable carriers, excipients or stabilizers such as those described in Remington's Pharmaceutical Sciences 16th edition, Osol, A. Ed. (1980) may be included in the formulation provided that they do not adversely affect the desired characteristics of the formulation. Acceptable carriers, excipients or stabilizers are nontoxic to recipients at the dosages and concentrations employed and include; additional buffering agents; co-solvents; anti-oxidants including ascorbic acid and methionine; chelating agents such as EDTA; metal complexes (e.g. Zn-protein complexes); biodegradable polymers such as polyesters; and/or salt-forming counterions. Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The formulation herein may also contain more than one protein as necessary for the particular indication being treated, preferably those with complementary activities that do not adversely affect the other protein. For example, where the antibody is anti-PDL1 (such as atezolizumab), it may be combined with another agent (e.g., a chemotherapeutic agent, and anti-neoplastic agent).

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The composition and formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. The formulations to be used for in vivo administration are generally sterile. Sterility may be readily accomplished, e.g., by filtration through sterile filtration membranes.

### VI. Articles of Manufacture or Kits

Further provided herein is an article of manufacture or a kit comprising an anti-PD-L1 antibody of the present disclosure (e.g., atezolizumab) and a package insert with instructions for using the anti-PD-L1 antibody according to any of the methods described herein.

In some embodiments, the anti-PD-L1 antibody is present in a pharmaceutically acceptable carrier. In some embodiments, the anti-PD-L1 antibody is provided in a unit dose. In some embodiments, the unit dose is 840 mg. In some embodiments, the unit dose is 840 mg, and the unit dose is provided in 14 mL of a solution (e.g., comprising the pharmaceutically acceptable carrier).

In some embodiments, the anti-PD-L1 antibody is present in a container. Suitable containers include, for example, bottles, vials, bags and syringes. The container may be formed from a variety of materials such as glass, plastic (such as polyvinyl chloride, polyethylene, or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### EXAMPLES

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### Overview

Immune checkpoint inhibition targeting programmed death-ligand 1 (PD-L1) or programmed death-1 (PD-1) has become an important approach in the treatment of multiple human cancers, as PD-L1 expression on tumor cells and tumor-infiltrating immune cells can inhibit anticancer immune responses (Chen et al., (2013) Immunicty doi:10.1016/j.immuni.2013.07.012). Atezolizumab, a humanized, engineered monoclonal immunoglobulin (Ig) G1 antibody, selectively targets PD-L1 to block interactions with its receptors to promote T-cell activation and reinvigorate and enhance anticancer activity, while leaving the interaction between PD-L2 and PD-1 intact (Chen et al., (2013) Immunicty doi:10.1016/j.immuni.2013.07.012; Chen et al., (2012) Clin Cancer Res doi:10.1158/1078-0432.CCR-12-1362; Herbst et al., (2014) Nature doi: 10.1038/nature14011). Atezolizumab is approved to treat certain types of locally advanced or metastatic non-small cell lung cancer (NSCLC) and urothelial carcinoma (UC) in the United States, Europe, and elsewhere, as well as locally advanced or metastatic triple-negative breast cancer (TNBC) and extensive-stage small-cell lung cancer (SCLC) in the United States (Tecentriq (atezolizumab) [package insert]. South San Francisco, CA: Genentech, Inc.; 2019. South San Francisco, CA, USA: Genentech, Inc; Tecentriq (atezolizumab) [summary of product characteristics]Welwyn Garden City, UK:&nbsp; Roche Registration Limited; 2018). The UC and NSCLC atezolizumab monotherapy indications as well as the NSCLC and SCLC atezolizumab combination therapy indications were first approved for IV infusions of 1200 mg q3w.

Identification of alternative dosing regimens that can be used interchangeably would offer patients greater convenience in their cancer treatment, particularly for combination regimens with diverse dosing requirements.

The following Examples describe studies to determine the exposure-response (ER) relationships between atezolizumab exposure and efficacy or safety in patients with advanced non-small cell lung cancer (NSCLC) or urothelial carcinoma (UC) and to identify alternative dosing regimens. In particular, the following Examples provide pharmacokinetic (PK) modeling and simulation predictions of atezolizumab monotherapy, based on integrated clinical pharmacology information available for atezolizumab in second-line (2L) non-small cell lung cancer (NSCLC) and first-line (1L) cisplatin-ineligible and 2L metastatic urothelial carcinoma (UC) from nine clinical studies (Table 1A and Table 1B).

The goals of these studies were to determine the atezolizumab ER relationship for efficacy and safety and to apply this knowledge, along with population PK (popPK) simulations and the known safety profile of atezolizumab, to identify alternative dosing regimens.

The results described herein suggest that atezolizumab exposure and thus exposure-response (ER) relationships of the approved 1200-mg q3w dosing regimen (administered as an intravenous infusion over 60 minutes for the first administration, and then, if tolerated by the patient, subsequent infusions administered over 30 minutes) are comparable to the 1680-mg q4w and 840 q2w dosing regimens (administered as an intravenous infusion over 60 minutes for the first administration, and then, if tolerated by the patient, subsequent infusions administered over 30 minutes) disclosed herein. Safety analyses and immunogenicity data based on data from Study PCD4989g, Study G028915 (OAK), and Study G029294 (IMvigor211) are also in support of the new 840-mg q2w and 1680-mg q4w dosing regimens.

TABLE 1A
Summary of Atezolizumab Studies Conducted in Monotherapy Settings.

| | | | N | |
|---|---|---|---|---|
| | | | Enrolled/PK | |
| | | | evaluable | |
| | | | (Total | Design/Dose/Primary |
| | Phase | Indication | 2938/2900)^{b} | Clinical Endpoint |
| PCD4989g (GO27831) | 1 | Multiple | 481/473 | Dose-escalation/ |
| “A Phase I, Open-Label, Dose- | | solid | | up to 20 mg/kg q3w/ |
| Escalation Study of the Safety | | tumor | | PK and safety |
| and Pharmacokinetics of | | | | |
| MPDL3280A Administered | | | | |
| Intravenously as a Single Agent | | | | |
| to Patients with Locally | | | | |
| Advanced or Metastatic Solid | | | | |
| Tumors or Hematologic | | | | |
| Malignancies”. 2015. Report | | | | |
| No. 1064914. | | | | |
| JO28944 | 1 | Multiple | 6/6 | Dose-escalation/ |
| “Phase I Clinical Study of | | solid | | 10 mg & 20 mg q3w/ |
| MPDL3280A in Patients with | | tumor | | PK and safety |
| Advanced Solid Tumors”. | | | | |
| 2015. Report No. 1067192. | | | | |
| IMvigor210 (GO29293) | 2 | IL^{a} & 2L | 438/427 | 1L, 2L cohorts/ |
| “A Phase II, Multicenter, | | mUC | | 1200 mg q3w/ |
| Single-Arm Study of | | | | ORR |
| MPDL3280A in Patients with | | | | |
| Locally Advanced or Metastatic | | | | |
| Urothelial Bladder Cancer”. | | | | |
| 2015. Report No. 1065272. | | | | |
| IMvigor211 (G029294) | 3 | 2L mUC | 467/455 | 2-arm study/ |
| “A Phase III, Open-Label, | | | | 1200 mg q3w vs. |
| Multicenter, Randomized Study | | | | vinflunine, |
| to Investigate the Efficacy and | | | | paclitaxel, or |
| Safety of Atezolizumab (Anti- | | | | docetaxel/OS |
| PD-L1 Antibody) Compared | | | | |
| with Chemotherapy in Patients | | | | |
| with Locally Advanced or | | | | |
| Metastatic Urothelial Bladder | | | | |
| Cancer After Failure with | | | | |
| Platinum-Containing | | | | |
| Chemotherapy”. 2017. Report | | | | |
| No. 1074426. | | | | |
| FIR (GO28625) | 2 | 1L, 2L+ | 138/137 | Single-arm study/ |
| “A Phase II, Single-Arm Study | | NSCLC | | 1200 mg q3w/ |
| of MPDL3280A in Patients | | | | ORR |
| with PD-L1-Positive Locally | | | | |
| Advanced or Metastatic Non- | | | | |
| Small Cell Lung Cancer”. 2015. | | | | |
| Report No. 1064438. | | | | |
| BIRCH (GO28754) | 2 | 1L, 2L+ | 667/654 | Single-arm study/ |
| “A Phase II, Multicenter, | | NSCLC | | 1200 mg q3w/ |
| Single-Arm Study of | | | | ORR |
| MPDL3280A in Patients with | | | | |
| PD-L1-Positive Locally | | | | |
| Advanced or Metastatic Non- | | | | |
| Small Cell Lung Cancer”. 2015. | | | | |
| Report No. 1066811. | | | | |
| POPLAR (GO28753) | 2 | 2L | 144/142 | 2-arm study/ |
| “A Phase II, Open-Label, | | NSCLC | | 1200 mg q3w vs |
| Multicenter, Randomized Study | | | | docetaxel/ |
| to Investigate the Efficacy and | | | | OS |
| Safety of MPDL3280A (Anti- | | | | |
| PD-L1 Antibody) Compared | | | | |
| with Docetaxel in Patients with | | | | |
| Non-Small Cell Lung Cancer | | | | |
| After Platinum Failure”. 2015. | | | | |
| Report No. 1065672. | | | | |
| OAK (GO28915) | 3 | 2L | 613/606 | 2-arm study/ |
| “A Phase III, Open-Label, | | NSCLC | | 1200 mg q3w vs. |
| Multicenter, Randomized Study | | | | docetaxel/ |
| to Investigate the Efficacy and | | | | OS |
| Safety of Atezolizumab (Anti- | | | | |
| PD-L1 Antibody) Compared | | | | |
| with Docetaxel in Patients with | | | | |
| Non-Small Cell Lung Cancer | | | | |
| After Failure with Platinum- | | | | |
| Containing Chemotherapy”. | | | | |
| 2016. Report No. 1070445. | | | | |

1L = first line;
2L = second-line;
2L+ = second line and beyond;
mUC = metastatic urothelial carcinoma;
NSCLC = non-small call lung cancer;
ORR = overall response rate;
q3w = every 3 weeks;
OS = overall survival;
PK = pharmacokinetic.
^{a}Cisplatin-ineligible patients
^{b}For randomized studies (i.e., IMvigor211, POPLAR, OAK), number enrolled includes patients enrolled into the atezolizumab arm

TABLE 1B
Summary of Atezolizumab Studies

| Study | PCD4989g | PCD4989g | OAK | IMvigor211 | Impassion130 |
|---|---|---|---|---|---|
| Population^{a} | NSCLC cohort | UC cohort | NSCLC | UC | Previously |
| | | | | | untreated |
| | | | | | locally |
| | | | | | advanced or |
| | | | | | metastatic |
| | | | | | TNBC |
| Clinical phase | 1 | 1 | 3 | 3 | 3 |
| Patients, n^{b} | 88 | 92 | Atezolizumab | 467 | 451 |
| | | | arm | (atezolizumab | (atezolizumab + |
| | | | 422^{c,d} | arm) | nab- |
| | | | 613^{e} | 464 | paclitaxel arm) |
| | | | Chemotherapy | (chemotherapy | 451 (placebo + |
| | | | arm | arm) | nab-paclitaxel |
| | | | 401^{d} | | arm) |
| | | | 612^{e} | | |
| Atezolizumab- | 87 | 90 | 414^{d} | 455 | 443 |
| exposed | | | 596^{e} | | (atezolizumab + |
| patients, n^{f} | | | | | nab-1 |
| | | | | | paclitaxel arm) |
| Atezolizumab | IV q3w | IV q3w | IV q3w | IV q3w | IV q2w |
| dose | | | 1200 mg | 1200 mg | 840 mg (in |
| | 1 mg/kg (n = 1) | 15 mg/kg (n = 84) | | | combination |
| | 10 mg/kg (n = 10) | 20 mg/kg (n = 1)^{g} | | | with nab- |
| | 15 mg/kg (n = 27) | 1200 mg (n = 5) | | | paclitaxel) |
| | 20 mg/kg (n = 49) | | | | |
| Patients for | | | | | |
| exposure- | | | | | |
| response | | | | | |
| analyses, n | | | | | |
| Exposure- | 87 | 90 | 414 | 451 | — |
| efficacy | | | | | |
| (ORR) | | | | | |
| TGI-OS | — | — | 388 | 382 | — |
| modeling | | | | | |
| Exposure- | 87 | 90 | 596 | 455 | — |
| safety | | | | | |

ITT intention to treat,
IV intravenous,
n number of patients,
NSCLC non-small cell lung cancer,
ORR, objective response rate,
PK pharmacokinetics,
q2w every 2 weeks,
q3w every 3 weeks,
TGI-OS tumor growth inhibition-overall survival,
TNBC triple-negative breast cancer,
UC urothelial carcinoma
^{a}Cohorts from PCD4989g and patients in OAK and IMvigor211 had locally advanced or metastatic disease. Patients in OAK and IMvigor211 had progression during or following platinum-containing chemotherapy
^{b}Refers to enrolled or ITT populations
^{c}Twenty-seven of the first 850 patients did not receive treatment
^{d}First 850 patients enrolled
^{e}All 1225 patients enrolled
^{f}Refers to patients who received ≥1 dose and for whom ≥1 evaluable PK sample was obtained
^{g}Patient's dose was incorrectly recorded as 20 mg/kg but was actually 15 mg/kg, which was used for deriving exposure

### Example 1

### Pharmacokinetic Properties of Atezolizumab Monotherapy

In this Example, the pharmacokinetic (PK) characteristics of atezolizumab are compared across eight atezolizumab studies conducted in monotherapy settings (see Table 1). Key PK characteristics such as Cₘᵢₙ, Cₘₐₓ, and AUC were calculated based on clinical studies using the fixed 1200-mg q3w dose and estimated for the fixed 1680-mg q4w and 840-mg q2w doses. Important patient characteristics were also analyzed as potential covariates.

Atezolizumab PK was linear over a dose range of 1 to 20 mg/kg of atezolizumab, including the fixed 1200 mg dose of atezolizumab. Atezolizumab PK appears comparable across studies as shown by similar observed Cₘₐₓ and Cₘᵢₙ for the same dose levels in Cycle 1 (Table 2).

TABLE 2
Summary Statistics for Atezolizumab Serum PK Parameters in Cycle 1 for PCD4989g,
JO28944, IMvigor210, IMvigor211, BIRCH, POPLAR, FIR, and OAK

| | PCD4989g | JO28944 | IMvigor210 | IMvigor211 | BIRCH | POPLAR | FIR | OAK |
|---|---|---|---|---|---|---|---|---|
| | GM | GM | GM | GM | GM | GM | GM | GM |
| | (% CV) | (% CV) | (% CV) | (% CV) | (% CV) | (% CV) | (% CV) | (% CV) |
| Study | n = 473 | n = 6 | n = 427 | n = 457 | n = 654 | n = 142 | n = 137 | n = 606 |
| | Cₘₐₓ (μg/mL) | | | | | | | |
| 10 mg/kg | 265 | 219 | — | — | — | — | — | |
| | (16) | (10.3) | | | | | | |
| | n = 36 | n = 3 | | | | | | |
| 15 mg/kg | 332 | — | — | — | — | — | — | |
| | (53) | | | | | | | |
| | n = 232 | | | | | | | |
| 1200 mg ^{a} | 405 | — | 360 | 334 | 397 | 326 | 405 | 345 |
| | (50) | | (23.2) | (34.2) | (67.2) | (25.1) | (31.7) | (153.5) |
| | n = 40 | | n = 406 | n = 408 | n = 624 | n = 139 | n = 135 | n = 561 |
| 20 mg/kg | 472 | 534 | — | — | — | — | — | |
| | (35) | (9.14) | | | | | | |
| | n = 145 | n =3 | | | | | | |
| | Cₘᵢₙ (μg/mL) | | | | | | | |
| 10 mg/kg | 54.1 | 36.8 | — | — | — | — | — | |
| | (25) | (3.63) | | | | | | |
| | n = 34 | n = 3 | | | | | | |
| 15 mg/kg | 67.1 | — | — | — | — | — | — | |
| | (73) | | | | | | | |
| | n = 214 | | | | | | | |
| 1200 mg ^{a} | 95.5 | — | 68.0 | 67.5 | 78.9 | 58.8 | 68.8 | 74.9 |
| | (51) | | (53.6) | (39.4) | (55.8) | (67.1) | (55.3) | (66.9) |
| | n = 30 | | n = 366 | n = 399 | n = 596 | n = 128 | n = 125 | n = 534 |
| 20 mg/kg | 91.1 | 113 | — | — | — | — | — | |
| | (36) | (10.1) | | | | | | |
| | n = 132 | n =3 | | | | | | |

Cₘₐₓ = maximum observed serum concentration;
Cₘᵢₙ = trough or minimum serum concentration;
CV = coefficient of variation;
GM = geometric mean;
PK = pharmacokinetics.
^{a} 1200 mg equivalent to 15 mg/kg (80 kg patient).

### Methodology

### Software

In some embodiments, in this Example and all other Examples provided herein, the following software tools and methods were used. Data set preparation, exploration, visualization, and analysis, including descriptive statistics, were performed using R version 3.4.3 and Comprehensive R Archive Network packages. Nonlinear mixed-effect modeling using the first-order conditional estimation algorithm with interaction (Non-Linear Mixed-Effect Modeling tool [NONMEM]version 7.3; ICON Development Solutions, Ellicott City, MD, USA) (Beal et al., (2011) NONMEM User's Guides. (1989-2011)) was used for Bayesian estimation of individual PK parameters. Logistic regression used the generalized linear model function in R with family “binomial” (variance=binomial; link=logit). Monte Carlo PK simulations were implemented using NONMEM version 7.3, and simulation data sets to assess were created using R.

popPK Model

The population PK (popPK) of atezolizumab was first assessed based on Phase I data from two clinical studies (the “Phase I popPK Model”): Study PCD4989g and Study JO28944. The Phase I popPK Model was subsequently subjected to an external validation for UC and NSCLC, separately, using PK data collected in IMvigor210 and IMvigor211 for UC and data collected in BIRCH, POPLAR, FIR, and OAK for NSCLC.

### Data Used in the Analysis

For the Phase I popPK Model, the pharmacokinetics of atezolizumab in serum were evaluated in 472 patients with 4563 samples from Studies PCD4989g and JO28944.

The popPK model was externally validated with atezolizumab serum PK samples from 423 patients (out of 429 treated, 98.6%) with 1251 samples from IMvigor210, 920 patients (out of 938 treated, 98.1%) with 3891 samples from BIRCH, POPLAR, and FIR, 596 patients (out of 608 treated, 98%) with 2754 samples from OAK and 455 patients (out of 467 treated, 97%) with 1939 samples from IMvigor211.

### Base Population PK Model

For the Phase I popPK Model, a nonlinear mixed-effects approach with the first-order conditional estimation method with interaction in NONMEM 7, version 7.3 (ICON, Maryland) was used to develop a base popPK model. Several candidate models were fit to the PK data. Various residual OMEGA matrix models were evaluated (block: accounting for correlation between IIVs; diagonal: IIVs independent from each other). Nonlinearity of pharmacokinetics was assessed using Michaelis-Menten models.

### Selection of Covariates

For the Phase I popPK Model, once the base model was finalized, an assessment of potential impact of covariates on primary PK parameters was performed.

In a first step, random effects of PK parameters generated by the population base PK model were plotted against covariates included in the analysis to qualitatively assess the extent of correlations. Scatterplots were used to examine the effect of continuous variables and boxplots were used to examine the effect of categorical variables.

In a second step, the formal covariate analysis involved a stepwise approach with forward additive inclusion and backward elimination, where the structural model was used as a baseline and the covariate model was made increasingly complex. After each model estimation, the covariates were evaluated to see which one resulted in the largest improvement in the objective function value (OFV) greater than the threshold (AOFV>−6.64 for one degree of freedom and a significance level of p<0.01). That covariate was added to the regression model for the structural parameter and the model was estimated. This process was repeated until all significant effects were accounted for. Then, the process was repeated in the opposite direction of backward deletion to eliminate covariates on parameters whose removal produced the smallest reduction in goodness-of-fit less than the threshold (ΔOFV≥+10.83 for one degree of freedom and 13.8 for two degrees of freedom at a significance level of p<0.001).

The following covariates were explored: gender, age, body weight (BW), Eastern Cooperative Oncology Group (ECOG) performance status, tumor burden, presence of liver metastasis, brain metastasis, visceral metastasis, and number of metastatic sites, liver function (AST, ALT, albumin, bilirubin), kidney function (creatinine clearance, estimated glomerular filtration rate (eGFR)), treatment emergent anti-drug antibodies (ADA).

Additional covariates were assessed after selection of statistically significant demographics or pathophysiological covariates by a forward selection approach and a backward elimination approach: Formulation (F01 versus F03), PD-L1 status (IC score and TC score), race, region, tumor type (urothelial carcinoma versus others and NSCLC versus others).

### External Validation: Urothelial Carcinoma

The Phase I popPK Model was used to derive the individual PK estimates based on atezolizumab observed concentration-time profiles in IMvigor210 and IMvigor211. A nonlinear mixed effects modeling approach was used with the Bayesian post-hoc estimation (MAXEVAL=0) in NONMEM 7, version 7.3 (ICON, Maryland).

A prediction-corrected visual predictive check (pcVPC) was performed based on the Phase I popPK Model, and observed peak (Cₘₐₓ) and trough (Cₘᵢₙ) in IMvigor210 and IMvigor211 were compared to corresponding predictive distributions. Individual estimates of IMvigor210 and IMvigor211 patient-level random effects were obtained and plotted versus baseline covariates to assess whether the Phase I popPK Model adequately captured covariate effects in IMvigor210 and IMvigor211.

### External Validation: Non-Small Cell Lung Cancer

The Phase I popPK Model was used to derive the individual PK estimates based on atezolizumab observed concentration-time profiles in BIRCH, POPLAR, FIR, and OAK. A nonlinear mixed effects modeling approach was used with the Bayesian post-hoc estimation (MAXEVAL=0) in NONMEM 7, version 7.3 (ICON, Maryland).

A pcVPC was performed based on the Phase I popPK Model, and observed peak (Cₘₐₓ) and trough (Cₘᵢₙ) in BIRCH, POPLAR, FIR, and OAK were compared to corresponding predictive distributions. Individual estimates of BIRCH, POPLAR, FIR, and OAK patient-level random effects were obtained and plotted versus baseline covariates to assess whether the Phase I popPK Model adequately captured covariate effects in BIRCH, POPLAR, FIR, and OAK patients.

### Results

Phase I popPK Model Overview

A noncompartmental analysis (NCA) indicated that doses ≥1 mg/kg display dose-proportional pharmacokinetics.

For the Phase I popPK Model, serum pharmacokinetics of atezolizumab across the two studies PCD4989g and JO28944 (dose range: 1-20 mg/kg q3w, including the fixed 1200 mg q3w dose of atezolizumab) was described by a linear two-compartment disposition model with first-order elimination. The estimated typical population total clearance of drug (CL) was 0.200 L/day and typical volume of distribution for the central compartment (V₁) was 3.28 L for a male patient with 40 g/L of albumin.

The typical volume of distribution under steady-state conditions (Vₛₛ) and terminal t_{1/2} estimates were 6.9 L and 27 days, respectively. Based on simulations in the current population, 90% of steady-state is attained after the following median (range) number of q3w cycles: 3 cycles (1-6), 2 cycles (1-4), and 3 cycles (1-5) for Cₘᵢₙ, Cₘₐₓ, and AUC, respectively. Inter-individual variability (IIV) was estimated to be 29%, 18%, and 34%, for CL, V₁, and volume of distribution in the peripheral compartment (V₂), respectively.

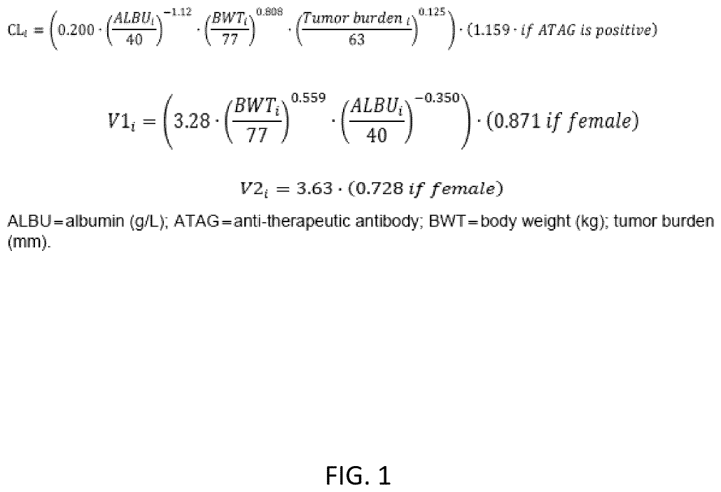

Statistically significant parameter-covariate relationships that were identified by the popPK model are provided in FIG. 1. The final popPK parameters are provided in Table 3.

TABLE 3
Final Population Pharmacokinetic Model Parameter Estimates for Atezolizumab.

| Parameters | Estimate | RSE (%) | Shrinkage (%) | Residual or IIV (%) |
|---|---|---|---|---|
| CL (L/day) | 0.200 | 2 | — | |
| V₁ (L) | 3.28 | 2 | — | — |
| V₂ (L) | 3.63 | 4 | — | — |
| Q (L/day) | 0.546 | 8 | — | — |
| Albumin on CL | −1.12 | 10 | — | — |
| ATA on CL | 0.159 | 25 | — | — |
| Tumor burden on CL | 0.125 | 17 | — | — |
| Body weight on CL | 0.808 | 8 | — | — |
| Albumin on V₁ | −0.350 | 21 | — | — |
| Body weight on V₁ | 0.559 | 8 | — | — |
| Gender (female) on V₁ | −0.129 | 16 | — | — |
| Gender (female) on V₂ | −0.272 | 16 | — | — |
| σ² Proportional residual error | 0.0433 | 7 | 9 | 21% |
| σ² Additive residual error | 16.6 | 39 | 9 | 4 μg/mL |
| ω² CL | 0.0867 | 9 | 9 | 29% |
| ω² V₁ | 0.0328 | 18 | 17 | 18% |
| ω² V₂ | 0.114 | 25 | 33 | 34% |
| Correlation CL.V₁ | 0.341 | — | — | — |
| Correlation CL.V₂ | −0.236 | — | — | — |
| Correlation V₁.V₂ | 0.434 | — | — | — |
| Objective function | 40748 | — | — | — |

ATA = anti-therapeutic antibody (equivalent to ant-drug antibody [ADA]);
CL = clearance;
IIV = inter-individual variability;
PK = pharmacokinetic;
Q = inter-compartmental clearance;
RSE = relative standard error;
V₁ = volume of distribution of central compartment;
V₂ = volume of distribution of peripheral compartment.
Note:
body weight = normalized to a 77-kg body weight;
albumin = normalized to 40 g/L;
tumor burden normalized to 63 mm;
ω² = variance of omega;
σ² = variance of sigma.

In patients who were positive for ADA, CL is estimated to be 16% higher than in patients without ADA. In females, volume of distribution would be 13% and 27% lower than in males for V₁ and V₂, respectively. No covariate induced more than 27% change from the typical PK model parameter for extreme values.

The popPK model estimated geometric mean accumulation ratio for Cₘᵢₙ, Cₘₐₓ, and AUC was 2.75, 1.46, and 1.91-fold, respectively, following multiple doses of 1200 mg atezolizumab q3w. In Study PCD4989g, the geometric mean accumulation ratio estimated from NCA ranged from 2.07 to 2.39 and from 1.21 to 1.41, for Cₘᵢₙ and Cₘₐₓ respectively, consistent with popPK model estimates. The observed extent of accumulation is in close agreement with that predicted based on the popPK reported t_{1/2} of 27 days dosed q3w.

The popPK model estimated geometric mean accumulation ratios for Cₘᵢₙ, Cₘₐₓ, and AUC were 3.05, 1.84, and 2.54-fold, respectively, following multiple doses of 840-mg atezolizumab q2w and 1.88, 1.35 and 1.72-fold, respectively following multiple doses of 1680-mg atezolizumab q4w.

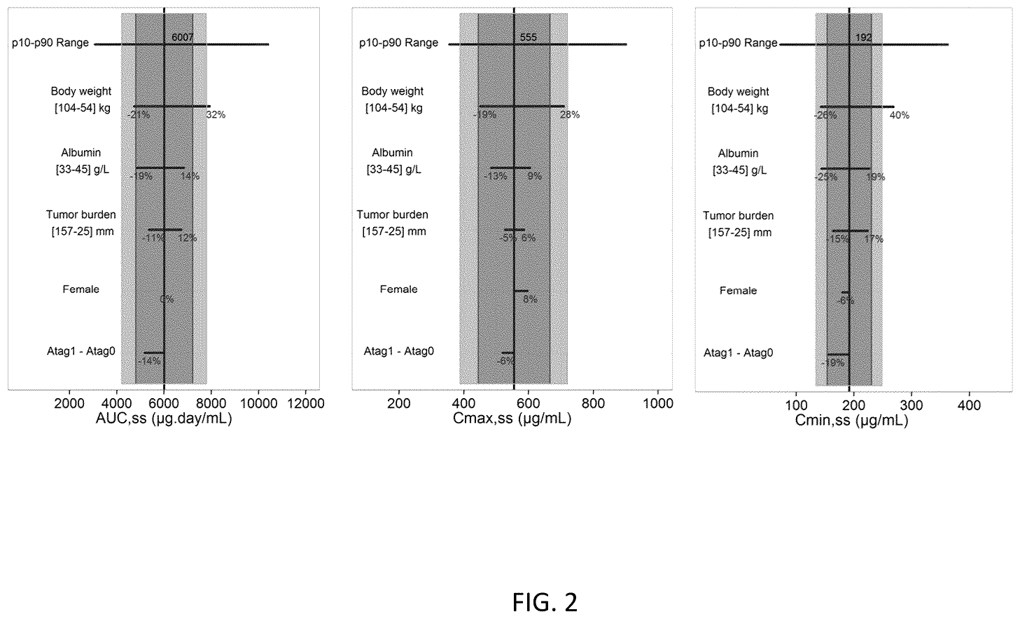

A sensitivity analysis was performed to examine the influence of the statistically significant covariates on steady-state exposure (area under the serum concentration time curve at steady-state [AUCₛₛ], maximum observed serum concentration at steady-state [C_{max,ss}], and minimum observed serum concentration at steady-state [C_{min,ss}]) of atezolizumab. FIG. 2 shows the isolated influence of each covariate (varying between the 10^{th} and 90^{th} percentiles for continuous covariates) on atezolizumab steady-state exposure after a 1200 mg dose q3w.

Overall, females have a moderately higher exposure compared to males.

Patients with low albumin tend to have a lower exposure with a larger effect on C_{min, ss}.

Baseline tumor burden and treatment-emergent positive ADA have a minor impact on exposure over the dose range investigated in this analysis (i.e., 1 to 20 mg/kg of atezolizumab q3w, or the fixed 1200 mg dose q3w).

Overall, no covariate effect induced more than 30% change in exposure from the typical patient (the typical patient is a male, treatment-emergent ADA-negative, weighing 77 kg, with an albumin level of 40 g/L and a tumor burden of 63 mm) except for BW when evaluated at the lowest extreme of weight (i.e., 10^{th} percentile). Patients with BW lower than 54 kg would have up to a 32%, 28%, 40% higher AUC,ₛₛ, C_{max,ss} or C_{min,ss}, respectively, than the typical patient.

None of these covariate effects would be expected to result in a C_{min,ss} that would be lower than a targeted serum concentration of 6 μg/mL. Further evaluation of the clinical significance, if any, of these relatively moderate effects on atezolizumab pharmacokinetics are described in the ER evaluations provided below (e.g., Examples 2-3).

Age was not identified as a significant covariate influencing atezolizumab pharmacokinetics based on patients with an age range of 21-89 years (n=472), and a median of 62 years of age. No clinically meaningful difference was observed in the pharmacokinetics of atezolizumab among patients <65 years (n=274), patients between 65-75 years (n=152) and patients >75 years (n=46). No dose adjustment based on age is required.

No clinically important differences in the CL of atezolizumab were found in patients with mild (eGFR 60 to 89 mL/min/1.73 m²; n=208) or moderate (eGFR 30 to 59 mL/min/1.73 m²; n=116) renal impairment compared to patients with normal (eGFR greater than or equal to 90 mL/min/1.73 m²; n=140) renal function. Few patients had severe renal impairment (eGFR 15 to 29 mL/min/1.73 m²; n=8).

There were no clinically important differences in the CL of atezolizumab between patients with mild hepatic impairment (bilirubin≤ULN and AST>ULN or bilirubin>1.0 to 1.5×ULN and any AST; n=71) and normal hepatic function (bilirubin and AST less than or equal to ULN; n=401). No data were available in patients with either moderate of severe hepatic impairments.

ECOG performance status or metastases (number of sites; brain, liver or visceral metastases) were not found to impact atezolizumab pharmacokinetics. After adjusting for significant demographic and pathophysiological covariate effects in the final model, a graphical exploration of patient-level random effect revealed that formulation did not impact atezolizumab pharmacokinetics nor did PD-L1 expression in either immune or tumor cells. Patients with UC or NSCLC did not show any trend of having different PK parameters than patients with other tumor types.

External Validation of the popPK Model for Urothelial Carcinoma

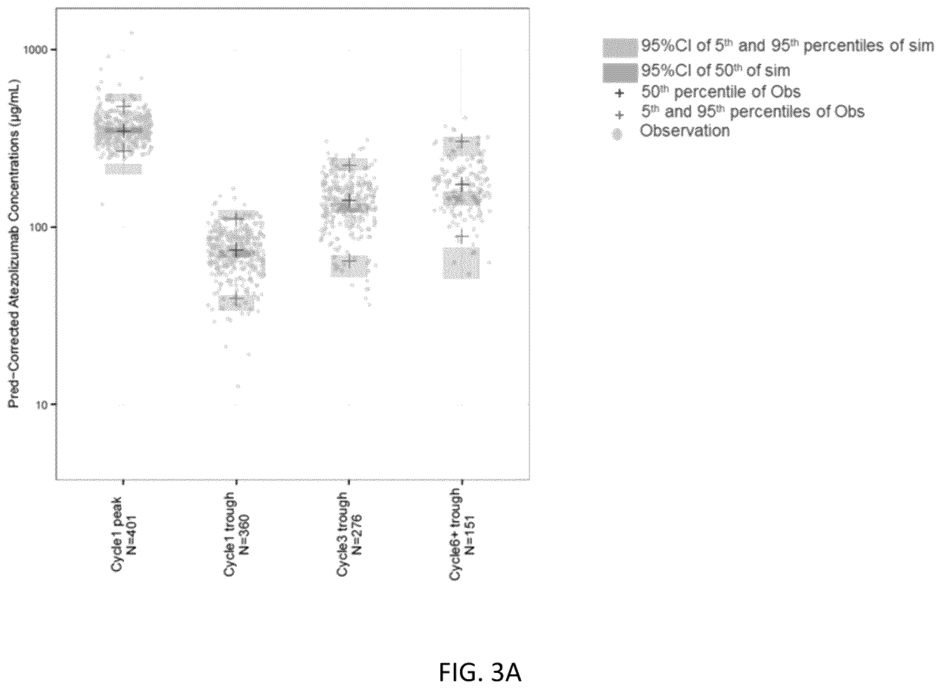

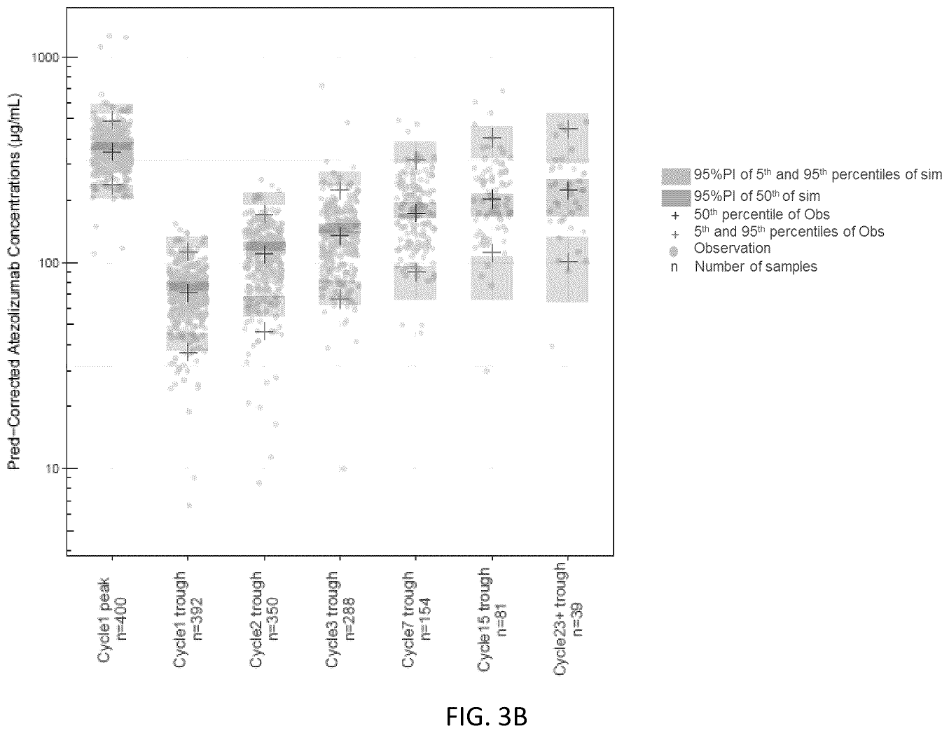

For external validation, PK data from IMvigor210 and IMvigor211 were simulated (1000 replicates) using actual dosing histories from IMvigor210 and IMvigor211 and the Phase I popPK Model. The prediction-corrected visual predictive check (pcVPC) of atezolizumab data for IMvigor210 and IMvigor211 are provided in FIG. 3A and FIG. 3B, respectively.

The pcVPC's for IMvigor210 and IMvigor211 suggested that the median, 95^{th} and 5^{th} percentiles of observed Cₘₐₓ and Cₘᵢₙ for all cycles were generally well captured, except the 95^{th} and 5^{th} percentiles of observed Cycle 1 Cₘₐₓ that were somewhat narrower than the corresponding predicted percentiles. There did not appear to be a consistent trend toward over-or under-prediction of atezolizumab exposure data upon multiple dosing. The pcVPC's suggested that the Phase I popPK Model was adequate to predict atezolizumab PK data in all patients from IMvigor210 and IMvigor211. Post-hoc estimation using the Phase I popPK Model was performed to obtain individual random-effects and PK parameters in patients from IMvigor210 and IMvigor211. Covariate effects in IMvigor210 and IMvigor211 data were consistent with those identified in the Phase I popPK Model; there did not appear to be any new covariate effect that was not previously identified in the Phase I popPK Model.

External Validation of the popPK Model for NSCLC

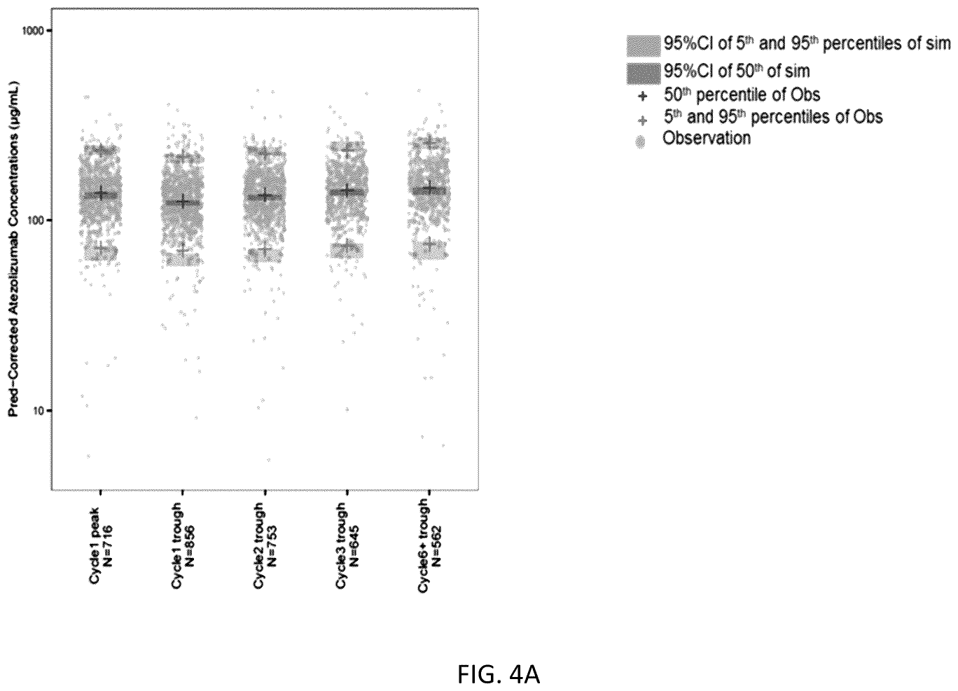

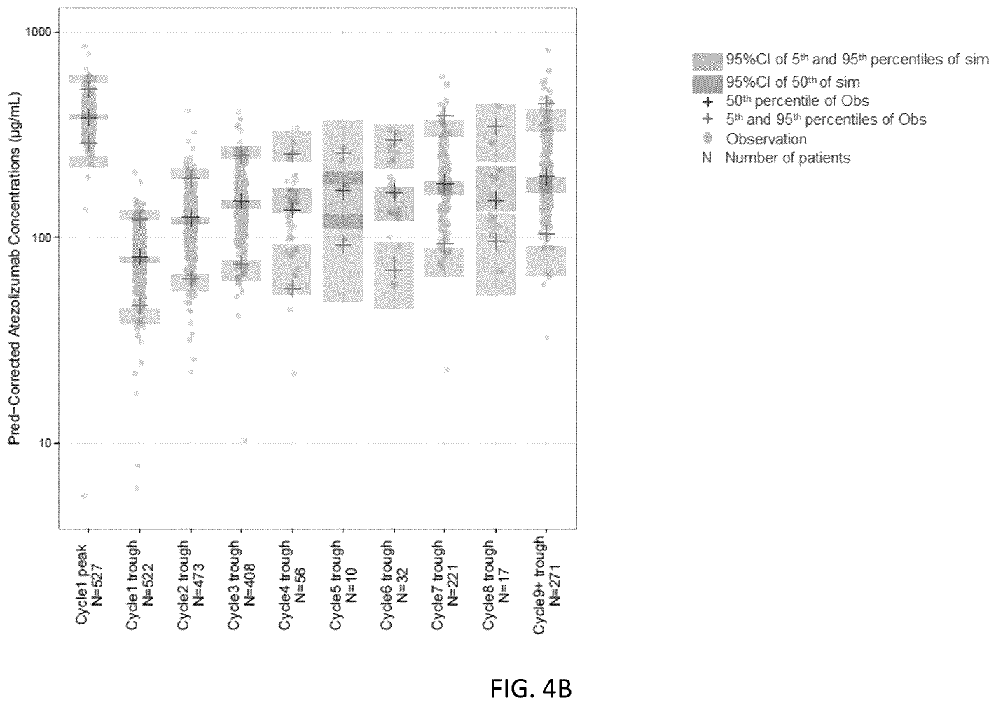

Similarly, PK data from BIRCH, POPLAR, FIR, and OAK were simulated (1000 replicates) using actual dosing histories from BIRCH, POPLAR, FIR, and OAK and the Phase I popPK Model. The pcVPCs of the BIRCH, POPLAR, and FIR atezolizumab pooled data, and OAK separately, are presented in FIG. 4A and FIG. 4B, respectively.

The pcVPC for all patients (BIRCH, POPLAR, and FIR studies combined, and OAK separately) suggested that the median, 95^{th}, and 5^{th} percentiles of observed Cₘₐₓ and Cₘᵢₙ for all cycles were generally well captured. There did not appear to be a consistent trend toward over-or under-prediction of atezolizumab exposure upon multiple dosing. The pcVPCs by study suggested that the Phase I popPK Model was adequate to predict atezolizumab PK data in BIRCH (all Cohorts) as well as in FIR (all Cohorts) and OAK. A trend to negative population-level predictions and residuals was observed for POPLAR, but this trend was resolved in individual predictions and residuals, indicating that the Phase I popPK Model allowed reliable and robust Bayesian estimation of individual parameter in all studies. Post-hoc estimation using the Phase I popPK Model was performed to obtain individual random-effects and PK parameters from patients enrolled in BIRCH, FIR, POPLAR, and OAK. Covariate effects in BIRCH, FIR, POPLAR, and OAK data were generally consistent with those identified in the Phase I popPK Model. Though there is a trend to faster CL and larger V₁ in POPLAR, exposure in POPLAR was only moderately impacted by those effects (i.e., AUC, Cₘₐₓ and Cₘᵢₙ were generally within 20% of estimates from BIRCH, FIR, and OAK). The relationship between random effect of CL and BW is characterized with a negative correlation coefficient, suggesting this relationship in patients with NSCLC may not be as steep as suggested by the Phase I popPK model. No new unexpected covariate effect was identified in BIRCH, FIR, POPLAR, and OAK. The combined atezolizumab PK data obtained in BIRCH, FIR, POPLAR, and OAK in patients with NSCLC patients are consistent with Phase I popPK Model estimates.

### Summary of Effects of Intrinsic Factors on PK of Atezolizumab

No dedicated studies of atezolizumab have been conducted in elderly patients. In the popPK analysis, age was not identified as a significant covariate influencing atezolizumab pharmacokinetics based on patients 21 to 89 years of age (n=472), and a median of 62 years of age. No clinically important difference was observed in the pharmacokinetics of atezolizumab among patients <65 years (n=274), patients between 65-75 years (n=152), and patients >75 years (n=46). No dose adjustment based on age is required. No dedicated studies of atezolizumab have been completed in pediatric patients.

In the popPK analysis, gender was identified as a statistically significant covariate on both V₁ and V₂, but not CL, based upon a dataset including 276 men (58.5%) and 196 women (41.5%). In females, volumes are 13% and 27% lower than in males for V₁ and V₂, respectively. For a typical female patient (weight normalized to 77 kg), there would be less than a 10% increase in AUCₛₛ, C_{max,ss}, or C_{min,ss} of atezolizumab compared to a typical male patient.

After adjusting for covariate effects in the final popPK model, race (Asian n=17, Black n=15, and White n=375) was not a significant covariate on the pharmacokinetics of atezolizumab and had no clinical relevance to atezolizumab CL.

No formal PK study has been conducted in patients with renal impairment. Based on the popPK analysis, no clinically important differences in the CL of atezolizumab were found in patients with mild (eGFR 60 to 89 mL/min/1.73 m²; n=208), or moderate (eGFR 30 to 59 mL/min/1.73 m²; n=116) renal impairment compared to patients with normal (eGFR greater than or equal to 90 mL/min/1.73 m²; n=140) renal function. Few patients had severe renal impairment (eGFR 15 to 29 mL/min/1.73 m²; n=8). No dose adjustment based on covariates related to renal function is required.

No formal PK study has been conducted in patients with hepatic impairment. Based on the popPK analysis, there were no clinically important differences in the CL of atezolizumab between patients with mild hepatic impairment (bilirubin≤ULN and AST>ULN or bilirubin>1.0 to 1.5×ULN and any AST; n=71) and normal hepatic function (bilirubin and AST less than or equal to ULN; n=401). No dose adjustment in patients with mild hepatic function impairment is required. No data were available in patients with either moderate or severe hepatic impairment.

Based on the popPK analysis, ECOG performance status, or metastases (number of sites; brain, liver, or visceral metastases) were not found to impact atezolizumab pharmacokinetics. Albumin and tumor burden were identified as statistically significant covariates on CL. None of these covariates resulted in more than 30% change in AUCₛₛ, C_{max,ss}, or C_{min,ss} from the typical patient when evaluated at extreme values (i.e., 10^{th} and 90^{th} percentiles) of the distribution of these covariates. After adjusting for covariate effects in the final popPK model, PD-L1 expression in either tumor-infiltrating immune cells (IC score) or tumor cells (TC score) did not impact atezolizumab pharmacokinetics. Patients with UC or NSCLC did not show any trend of having different PK parameters from patients with other tumor types.

### Summary of Effects of Extrinsic Factors on PK of Atezolizumab

In the popPK analysis, there was no effect of a change in Drug Product/formulation on the pharmacokinetics of atezolizumab. No PK drug-drug interaction studies have been conducted.

After adjusting for covariate effects in the final popPK model, region (Japan versus Spain versus France versus Great Britain versus USA) was not a significant covariate on the pharmacokinetics of atezolizumab and it had no clinical relevance on atezolizumab CL.

### Example 2

### Exposure-Efficacy Relationships for Atezolizumab in Urothelial Carcinoma and Non-Small Cell Lung Cancer

Exposure-response (ER) analyses were conducted to assess possible relationships between clinical efficacy and atezolizumab exposure for patient populations in each indication (UC or NSCLC) separately as well as pooled (UC and NSCLC).

### Methodology

### Overview of Pooled ER Analyses

Objective response rate, overall survival, and adverse events were evaluated vs pharmacokinetic (PK) metrics, as described below.

ER analyses were performed to inform any relationships between PK metrics and ORR, OS, grade 3 to 5 AE, and AESI endpoints evaluated in previous clinical studies based on cycle 1 data to minimize potential bias due to both confounding with baseline prognostic factors (Yang et al., (2013) J Clin Pharmacol doi: 10.1177/0091270012445206; Wang et al., (2014) Clin Pharmacol Ther doi: 10.1038/clpt.2014.24) and time-dependent variation in clearance that has been observed for atezolizumab and other checkpoint inhibitors (Tecentriq (atezolizumab) [package insert]. South San Francisco, CA: Genentech, Inc.; 2019. South San Francisco, CA, USA: Genentech, Inc.; Bi et al., (2019) Ann Oncol doi: 10.1093/annonc/mdz037; Bajaj et al., (2017) CPT Pharmacometrics Syst Pharmacol doi: 10.1002/psp4.12143; Li et al., (2017) J Pharmacokinet Pharmacodyn doi: 10.1007/s10928-017-9528-y; Liu et al., (2017) Clin Pharmacol Ther doi: 10.1002/cpt.656; Wang et al., (2017) Clin Pharmacol Ther doi: 10.1002/cpt.628). These analyses were conducted using pooled data from atezolizumab-treated patients with NSCLC or UC (from PCD4989g, OAK, and IMvigor211) for whom exposure data were available, except as noted below for overall survival (OS). Exploratory ER analyses were performed using cycle 1 maximum serum concentration (Cₘₐₓ), Cₘᵢₙ, and area under the concentration-time curve (AUC; time 0-21 days), as recommended (Liu et al., (2017) Clin Pharmacol Ther doi: 10.1002/cpt.656) to minimize the effect of response-dependent time-varying clearance observed previously for anti-PD-1 and anti-PD-L1 agents (Li et al., (2017) J Pharmacokinet Pharmacodyn doi: 10.1007/s10928-017-9528-y). AUC (time 0-21 days), Cₘₐₓ, and Cₘᵢₙ were derived at cycle 1 based on individual PK parameters estimated using cycle 1 data only and the previously developed popPK model (Stroh et al., (2017) Clin Pharmacol Ther doi: 10.1002/cpt.587). The efficacy endpoints evaluated were investigator-assessed confirmed Response Evaluation Criteria in Solid Tumors version 1.1 (RECIST 1.1) objective response rate (ORR; secondary endpoint in all studies) and OS (primary endpoint in OAK and IMvigor211). ORR analyses used data from atezolizumab-treated patients with NSCLC or UC in PCD4989g, OAK (first 850 randomized patients), and IMvigor211, whereas OS analyses used data from OAK (first 850 randomized patients) and IMvigor211 only. The safety endpoints evaluated included adverse events (AEs) of grades 3 to 5 per National Cancer Institute Common Terminology Criteria for Adverse Events version 4 and Medical Dictionary for Regulatory Activities version 20.1 (primary endpoint in PCD4989g, also evaluated in OAK and IMvigor211) and AEs of special interest (AESIs; evaluated in all studies). AESIs, conditions suggestive of autoimmune disorder, were defined previously (Petrylak et al., (2018) JAMA Oncol doi: 10.1001/jamaoncol.2017.5440).

ORR and AEs were evaluated as binary endpoints (yes/no) and studied vs. exposure as a continuous variable using logistic regression. The Wald test P value was reported for each logistic regression, along with proportions/frequencies and their 95% CIs computed for quartiles of exposure. For OS data, to mitigate confounding factors between patients' baseline information and atezolizumab clearance and exposure, TGI-OS modeling (Bruno et al., (2014) Clin Pharmacol Ther doi: 10.1038/clpt.2014.4; Claret et al., (2018) Clin Cancer Res doi: 10.1158/1078-0432.CCR-17-3662) was performed. To be evaluable in this analysis (TGI evaluable), patients needed to have ≥1 posttreatment sum of longest diameters (SLD) assessment. The impact of individual baseline prognostic factors and TGI metrics (estimated tumor shrinkage and tumor growth rates in a biexponential longitudinal model of the SLD of the target lesions per RECIST 1.1) on OS were explored using Kaplan-Meier and Cox regression analyses, and a parametric multivariate regression TGI-OS model was built. The final TGI-OS model was validated by simulation in its ability to describe OS distributions and hazard ratios (HRs) compared with a control in different subgroups (notably by exposure quartiles). For the HR simulations, TGI metric estimates and baseline covariates for control patients were taken from previous analyses (Claret et al., (2018) Clin Cancer Res doi: 10.1158/1078-0432.CCR-17-3662; Bruno et al., (2018) J Clin Oncol doi: 10.1200/JCO.2018.36.5_suppl.62). Exposure metrics were tested on the final multivariate model after adjustment for confounding with prognostic factors. A “tumor type” factor was incorporated in the model if appropriate.

### ER Analysis and OS Modeling for Urothelial Carcinoma

The atezolizumab exposure-efficacy relationship for patients with mUC was individually assessed in two studies, IMvigor210 and IMvigor211. In both studies, Cycle 1 exposure metrics were used to accommodate the slight time- and response-dependent change in clearance observed previously with anti-PD-1 and anti-PD-L1 antibodies. For IMvigor210, the primary endpoint, objective response rate (ORR), was used as the efficacy metric. For IMvigor211, ORR and the primary endpoint, OS, were used in the exposure-efficacy assessment.

Atezolizumab exposure metrics (AUC, Cₘₐₓ, and Cₘᵢₙ) were derived at Cycle 1 from simulated PK profiles based on individual PK parameters. Atezolizumab AUCₛₛ was calculated as Starting Dose/CL.

ORR was characterized by responder status (Yes/No). The proportions of responders and 95% CI were computed for intervals of exposure with an equivalent number of individuals (e.g., quartiles). For each correlation, a logistic regression was performed and the Wald test p-value for exposure effect on the probability of response in the logistic regression was reported.

To mitigate confounding between patients' prognostic factors and atezolizumab clearance and exposure, tumor growth inhibition-overall survival (TGI-OS) modeling (disease modeling) was performed. Patient-level tumor growth inhibition (TGI) metrics were estimated using parameter estimates from a longitudinal tumor size model previously described by Stein et al., (2011) Clin Cancer Res 18:907-917 and implemented by Claret et al., (2013) J Clin Oncol 31:2110-2114 that was fit to evaluable patients. The growth rate, characterized by the growth rate constant (KG) for individual patients was estimated by post hoc empirical Bayesian estimation from the TGI model.

The multivariate parametric OS model was developed with KG and other covariates. A “full” OS model was built by first including all significant covariates from a univariate analysis (Cox, p<0.05) and then a backward stepwise elimination was carried out using a cutoff of p<0.01. The OS model was evaluated in its ability to simulate observed OS distributions and hazard ratio (HR) in IMvigor211. (Stein et al., (2011) Clin Cancer Res 18:907-917, Claret et al., (2013) J Clin Oncol 31:2110-2114).

### ER Analysis and OS Modeling for NSCLC

The Independent Review Facility (IRF)-assessed ORR per Response Evaluation Criteria in Solid Tumors (RECIST) v1.1 from BIRCH and the OS and investigator-assessed ORR per RECIST v1.1 from POPLAR and OAK were considered in the exposure-efficacy assessment. The IRF-assessed ORR per RECIST v1.1 was the primary endpoint in BIRCH, and OS was the primary endpoint in POPLAR and OAK. For BIRCH, the analysis population in the exposure-efficacy assessment was patients with second-line and beyond (2L+) TC2/3 or IC2/3 NSCLC who represented the intent-to-treat population in Cohorts 2 and 3. For POPLAR and OAK, the analysis population in the exposure-efficacy assessment was a PD-L1-unselected NSCLC patient population (i.e., all corners). The IRF-assessed ORR per RECIST v1.1 from BIRCH and investigator-assessed ORR per RECIST v1.1 from POPLAR and OAK were analyzed separately for ER.

The efficacy endpoint ORR was characterized by responder status (Yes/No). The proportions of frequency and 95% CI were computed for intervals of exposure with an equivalent number of individuals (e.g., quartiles). For each correlation, a logistic regression was performed and the Wald test p-value for exposure effect in the logistic regression was reported.

### p(ORR)˜Exposure

Where, p(ORR) is the probability of the objective response and exposure is an atezolizumab exposure metric.

To mitigate confounding between patients' prognostic factors and atezolizumab clearance and exposure, TGI-OS modeling (disease modeling) was performed. Patient-level TGI metrics were estimated using parameter estimates from a longitudinal tumor size model as previously described by Stein et al., (2011) Clin Cancer Res 18:907-917 and implemented by Claret et al., (2013) J Clin Oncol 31:2110-2114 that was fit to evaluable patients. The growth rate, characterized by the KG for individual patients was estimated by post hoc empirical Bayesian estimation from the TGI model.

The multivariate parametric OS model was developed using regression analysis with KG and other covariates. A “full” OS model was built by first including all significant covariates from a univariate analysis (Cox, p<0.05) and then a backward stepwise elimination was carried out using a cutoff of p<0.01. The OS model was evaluated in its ability to simulate observed OS distributions and HR in POPLAR and OAK. The model was then simulated to characterize the (non-confounded) ER due to KG on OS (Stein et al., (2011) Clin Cancer Res 18:907-917, Claret et al., (2013) J Clin Oncol 31:2110-2114).

### Pooled (UC and NSCLC) ER Analysis and OS Modeling

The atezolizumab exposure-efficacy relationship was assessed in a pooled analysis of patients with either mUC or NSCLC in studies PCD4989g, IMvigor211 and OAK. The efficacy endpoints considered for the exposure-response analysis were ORR (investigator-assessed using RECIST v1.1) in all atezolizumab treated mUC and NSCLC patients in Studies PCD4989g, IMvigor211, and OAK and OS in all atezolizumab treated mUC and NSCLC patients in Studies IMvigor211 and OAK. Cycle 1 exposure metrics were used to accommodate the slight time- and response-dependent change in clearance observed previously for anti PD1 and PD-L1 antibodies.

The efficacy endpoint ORR was characterized by responder status (Yes/No). The proportions of responders and 95% CI were computed for intervals of exposure with an equivalent number of individuals (e.g., quartiles). For each correlation, a logistic regression was performed and the Wald test p-value for exposure effect in the logistic regression was reported.

To mitigate confounding between patients' prognostic factors and atezolizumab clearance and exposure, TGI-OS modeling (disease modeling) was performed. Patient-level TGI metrics were estimated using parameter estimates from a longitudinal tumor size model that was fit to evaluable patients as previously described by Stein et al., (2011) Clin Cancer Res 18:907-917 and implemented by Claret et al., (2013) J Clin Oncol 31:2110-2114. The growth rate, characterized by the KG for individual patients was estimated by post hoc empirical Bayesian estimation from the TGI model.

The multivariate parametric OS model was developed with KG and other covariates. A “full” OS model was built by first including all significant covariates from a univariate analysis (Cox, p<0.05) and then a backward stepwise elimination was carried out using a cutoff of p<0.01. The OS model was evaluated in its ability to simulate observed OS distributions and HR in IMvigor211 and OAK (Stein et al., (2011) Clin Cancer Res 18:907-917, Claret et al., (2013) J Clin Oncol 31:2110-2114).

Atezolizumab exposure metrics (AUC, Cₘₐₓ, and Cₘᵢₙ) were derived at Cycle 1 from simulated PK profiles based on individual PK parameters.

### Results

### Urothelial Carcinoma ER Analysis and OS Modeling Results

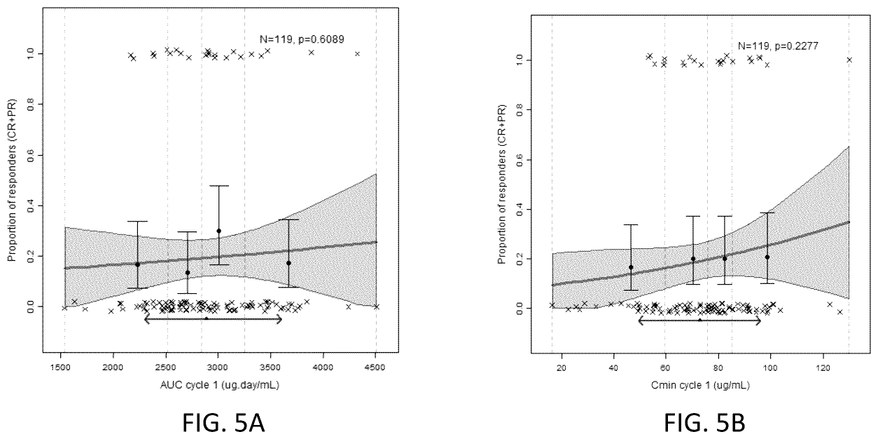

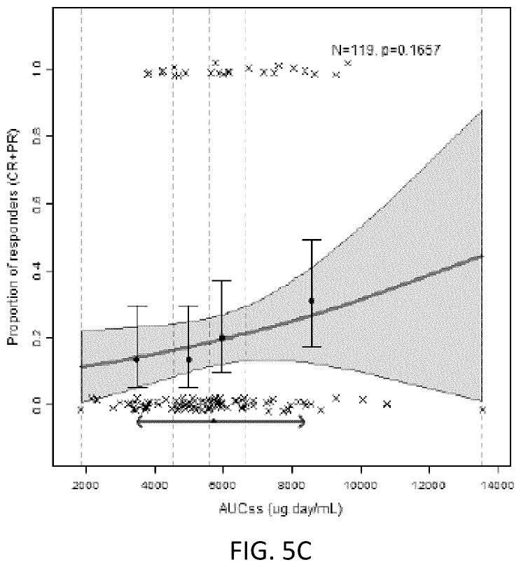

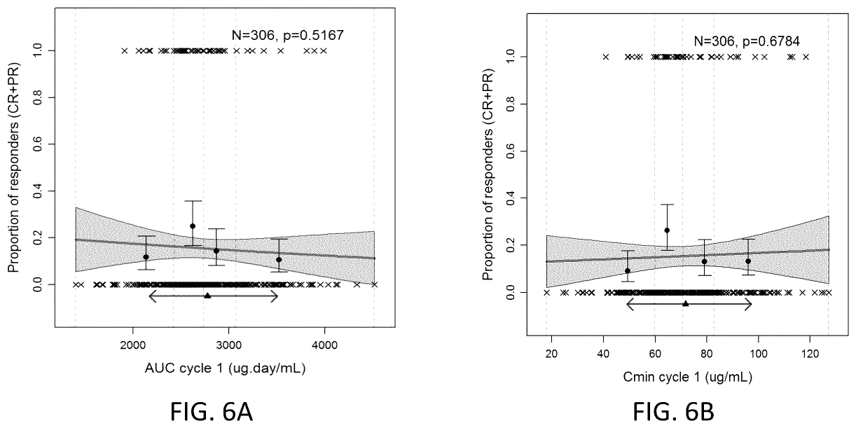

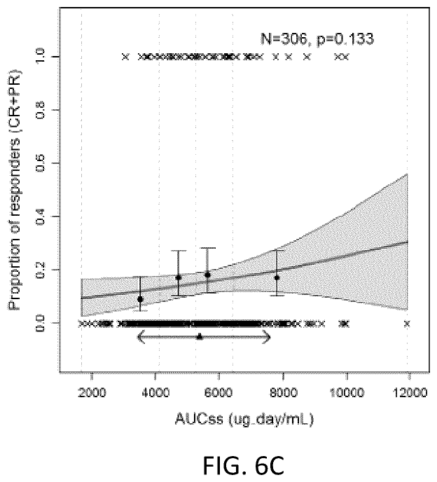

There was no statistically significant ER relationship between probability of response and atezolizumab exposure with any of the exposure metrics considered patients in IMvigor210 (Cohorts 1 and 2) treated with atezolizumab 1200 mg q3w. The relationships between ORR and cycle 1 AUC, cycle 1 Cₘᵢₙ, and AUCₛₛ for patients in IMvigor210 receiving atezolizumab 1200 mg q3w are provided in FIGS. 5A-5C for patients with 1L cisplatin-ineligible urothelial carcinoma and in FIGS. 6A-6C for patients with 2L urothelial carcinoma.

Similarly, for patients in IMvigor211, no statistically significant ER relationships (cycle 1 AUC) were identified with ORR following atezolizumab 1200 mg q3w (FIG. 7). A statistically significant ER relationship was initially identified with OS using a univariate analysis. However, the exposure (AUC Cycle 1) was no longer significant (p>0.01) when tested on the final multivariate model (p=0.0812), indicating that the multivariate OS model adjusted for the confounding in the AUC-OS relationship seen in the univariate analysis. None of the TGI metrics, Log(KG) or Log(KS), were significantly correlated with AUC Cycle 1.

None of the changes in atezolizumab exposure associated with the statistically significant covariates identified with the popPK model (see Example 1) would be expected to be clinically meaningful or require dose adjustment. Accordingly, the reduction in atezolizumab exposure when evaluated at extreme values (i.e., 90^{th} percentile) of weight compared to the typical patient following administration of the atezolizumab 1200-mg q3w flat dose would not be expected to be clinically meaningful or require dose adjustment by BW.

### Non-Small Cell Lung Cancer ER Analysis and OS Modeling Results

For patients treated with atezolizumab 1200 mg q3w in BIRCH and OAK, there was a statistically significant ER relationship between probability of response and atezolizumab exposure with at least one of the exposure metrics considered.

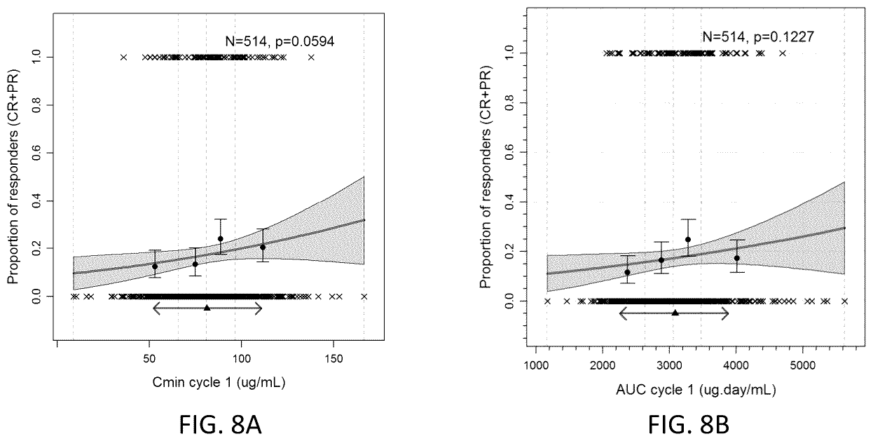

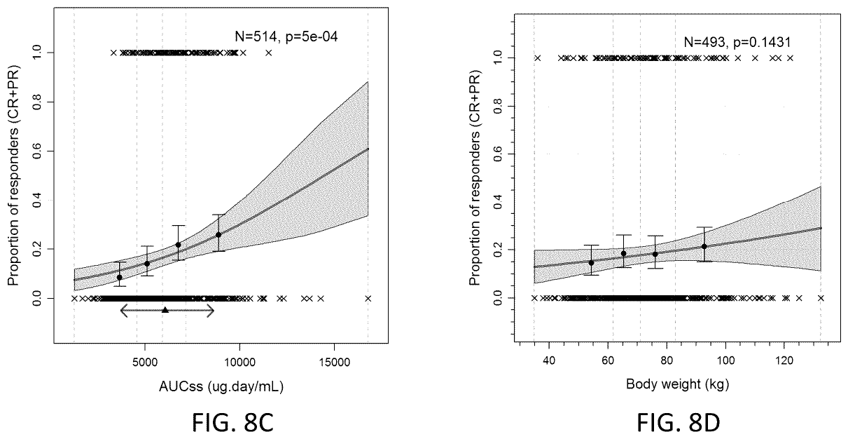

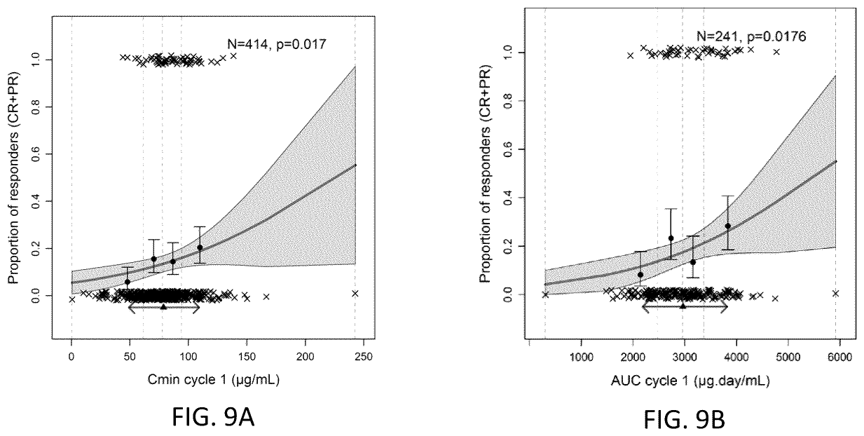

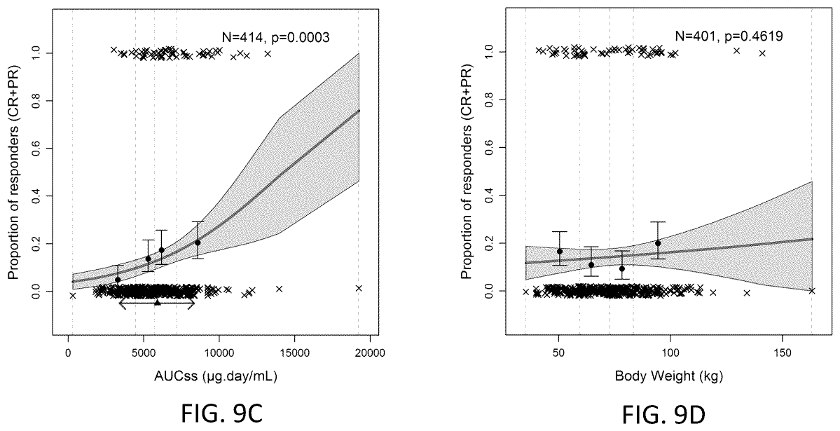

For BIRCH and OAK, of the exposure metrics associated with a trend toward increased probability of response with atezolizumab exposure, the p-values associated with AUCₛₛ (p=0.0005343 and p<0.0003, respectively) were the lowest. For BIRCH, the logistic regressions for cycle 1 Cₘᵢₙ, cycle 1 AUC, AUCₛₛ, and body weight are provided in FIGS. 8A-8D, respectively. For OAK, the logistic regressions for cycle 1 Cₘᵢₙ, cycle 1 AUC, AUCₛₛ, and body weight are provided in FIGS. 9A-9D, respectively.

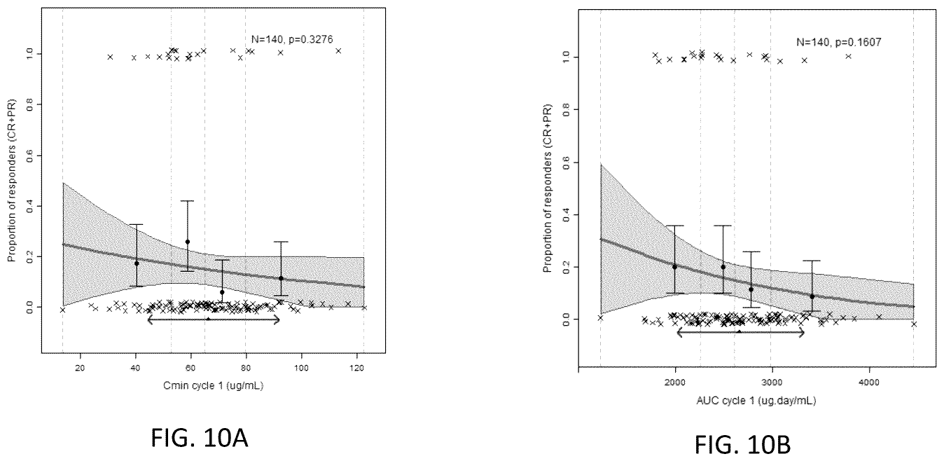

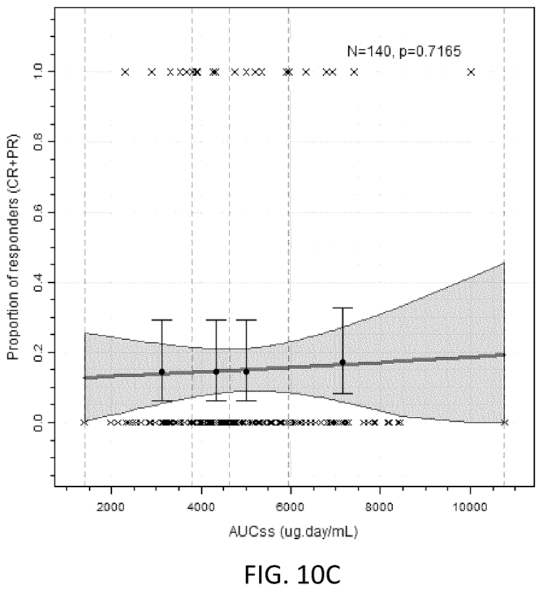

For patients treated with atezolizumab 1200 mg q3w in POPLAR, there was no statistically significant ER relationship between probability of response and atezolizumab exposure with any of the exposure metrics considered. The logistic regressions for cycle 1 Cₘᵢₙ, AUC Cycle 1, and AUCₛₛ and are provided in FIGS. 10A-10C, respectively. A sensitivity analysis was performed in patients with 2L/3L TC2/3 or IC2/3 NSCLC in POPLAR, which further suggested no statistically significant ER relationship between probability of response and atezolizumab exposure.

A model-based evaluation of OS was also considered in the exposure-efficacy assessments in POPLAR and OAK. For both POPLAR and OAK, the Log of KG (Log KG) and a range of patient prognostic factors explained the atezolizumab effect on OS.

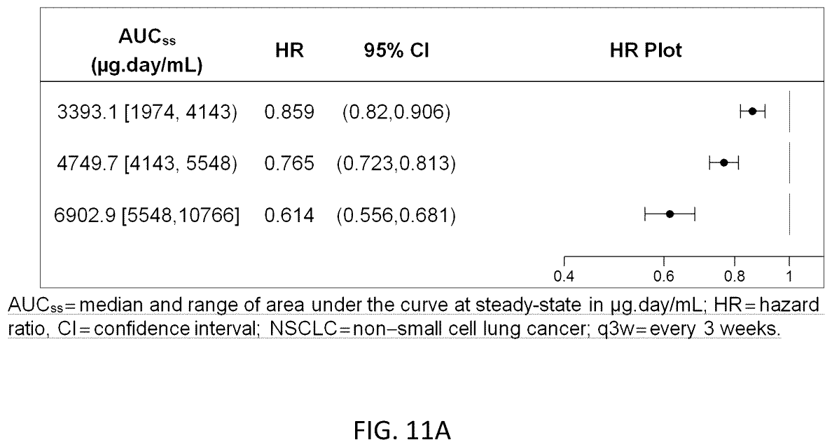

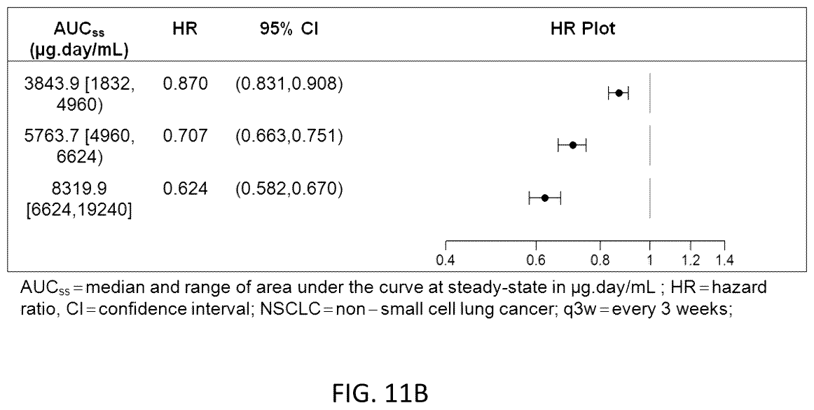

Specifically, for the POPLAR multivariate OS model, the number of metastatic sites, albumin level, and the log KG explained the atezolizumab effect on OS. The log of KG was correlated with atezolizumab AUCₛₛ. The multivariate OS model was used to infer ER on OS based on the ER on log KG. HRs comparing atezolizumab to docetaxel OS in each group of AUCₛₛ tertiles were simulated. Simulation of the OS model after correcting for the imbalance of prognostic factors (number of metastatic sites and albumin level) across AUCₛₛ tertiles and docetaxel groups suggested that all patients would benefit from atezolizumab treatment (HR estimate [95% prediction interval]=0.859 [0.820,0.906]in low exposure patients [1st tertile]; 0.614 [0.556,0.681]in high exposure patients [3rd tertile]) (FIG. 11A).

Specifically, for the OAK multivariate OS model, the baseline sum of longest diameter (BSLD), albumin level, ECOG performance status >0, lactic dehydrogenase (LDH) level and log KG explained the atezolizumab effect on OS. The log KG was correlated with atezolizumab AUCss. The multivariate OS model was used to infer ER on OS on the basis of the ER on the log KG. HRs that compare atezolizumab to docetaxel OS in each group of AUCss tertiles were simulated. Simulation of the OS model after correction of the imbalance of prognostic factors (baseline BSLD, albumin, ECOG performance status, and LDH level) across AUCss tertiles and docetaxel groups suggested that all patients would benefit from treatment with atezolizumab (HR estimate [95% prediction interval]=0.870 [0.831,0.908]in low exposure patients [1st tertile]; 0.624 [0.582,0.670]in high exposure patients [3rd tertile](FIG. 11B).

In BIRCH, simulation of the ER relationship for AUCss suggested a decrease in the ORR (estimate [prediction interval]) from 0.16 (0.13, 0.20) to 0.13 (0.10, 0.17) for patients with the median and 25th percentile of AUCss, respectively. Given the overlapping confidence intervals (CIs), the small decrease in ORR and the lack of correlation between efficacy as measured by ORR compared with OS in this treatment setting, this change in ORR is considered unlikely to be clinically meaningful. Further, as time- and response-dependent decreases in clearance have been observed with anti PD-1 and PD-L1 inhibitors, the use of AUCss as the exposure metric in the exposure-response analyses may overestimate the potential relationship between exposure and ORR.

In OAK, a simulation of the ER relationship for AUCss suggested a decrease in the ORR (estimate [prediction interval]) from 0.13 (0.10, 0.16) to 0.10 (0.07, 0.14) for patients with the median and 25th percentile of AUCss, respectively. Given the overlapping CIs, the small decrease in ORR and the lack of correlation between efficacy as measured by ORR compared with OS in this treatment setting, this change in ORR is also considered unlikely to be clinically meaningful. In POPLAR, there was no statistically significant ER relationship with ORR.

Since no single effect in the Phase I popPK Model (i.e., BW, gender, ADA, albumin, and tumor burden) was associated with a >25% decrease in AUCss, none of the changes in AUCss associated with the statistically significant covariates identified with the popPK model would be expected to exceed the change in ORR at the 25th percentile of AUCss or the HR for OS at the lowest tertile of atezolizumab exposure for BIRCH (FIG. 8C) or for OAK (FIG. 9C). As with UC, none of the fold-changes in atezolizumab exposure associated with these statistically significant covariates identified with the popPK model would be expected to be clinically meaningful or require dose adjustment.

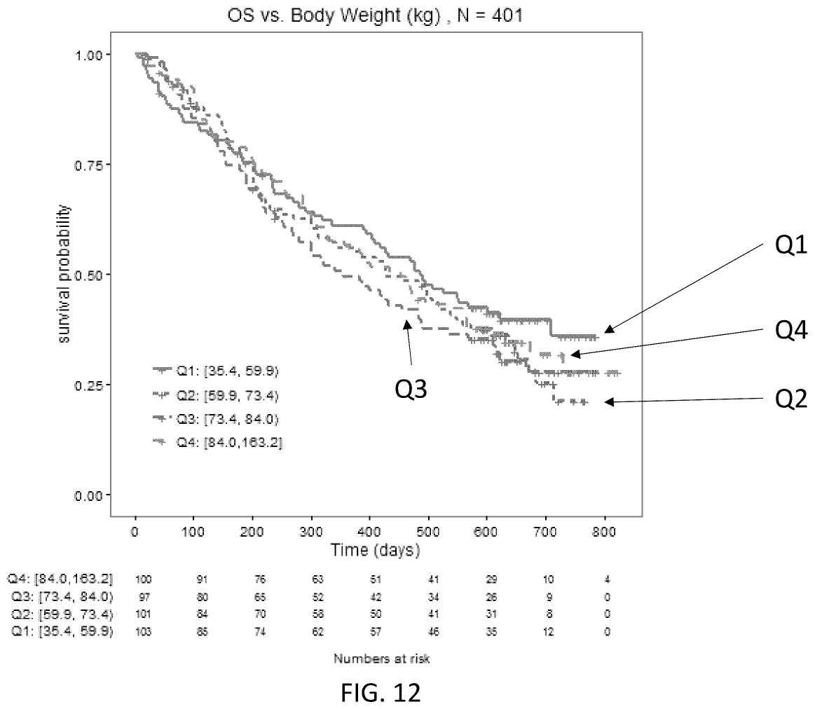

Accordingly, the reduction in atezolizumab exposure when evaluated at extreme values of weight compared with the typical patient (i.e., 21% decrease in AUCₛₛ) following administration of the atezolizumab 1200 mg q3w flat dose is considered unlikely to require dose adjustment or adjustment by BW. The observation that there is no statistically significant relationship of ORR with BW for BIRCH (FIG. 8D) and OAK (FIG. 9D) further supports selection of the flat 1200 mg q3w dose of atezolizumab. Simulations suggest that administration of a weight-based 15 mg/kg atezolizumab dose to patients who would otherwise be at the lowest quartile of atezolizumab exposure following a fixed 1200 mg atezolizumab dose would not improve ORR in these patients. Further support for the flat 1200-mg q3w dose of atezolizumab comes from OAK, where Kaplan-Meier plots of OS by quartiles of BW (FIG. 12) suggest that heavier weight patients have similar OS to lighter weight patients.

### Pooled (NSCLC and UC) ER Analysis and OS Modeling Results

ORR in mUC and NSCLC from patients treated with atezolizumab in PCD4989g, IMvigor211 and OAK was evaluated in the exposure-efficacy assessment. The population comprised mUC and NSCLC patients (1042 atezolizumab-treated patients with exposure data). The ORR (proportion of confirmed CR and PR; investigator assessed) per RECIST v1.1 in the analysis population was 15.7% (164 responders out of 1042 patients with exposure data). There was no difference in ORR in mUC (15.9%, N=541 patients) and NSCLC (15.6%, N=501 patients), therefore, tumor type was not included in the logistic regression models.

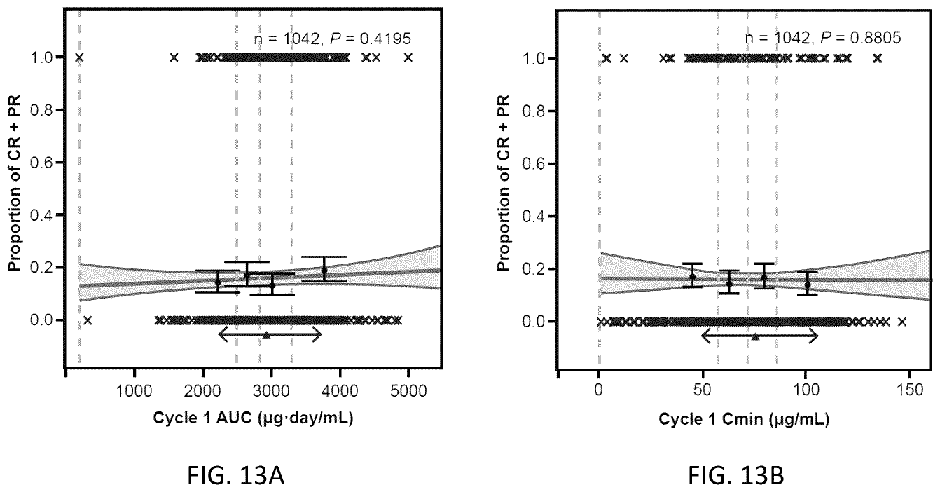

As shown in Table 4 and FIGS. 13A-13B, there was no statistically significant ER relationship between probability of response and atezolizumab exposure with any of the exposure metrics considered (AUC cycle 1, Cₘₐₓ cycle 1, and Cₘᵢₙ cycle 1).

TABLE 4
Summary of logistic regression results for the probability of
response vs. exposure in pooled mUC and NSCLC patients.

| Exposure Metric (units) | N | p-value | Sign |
|---|---|---|---|
| AUC cycle 1(μg · day/mL) | 1042 | 0.4195 | NA |
| Cₘₐₓ cycle 1 (μg/mL) | 1042 | 0.7816 | NA |
| Cₘᵢₙ cycle 1 (μg/mL) | 1042 | 0.8805 | NA |

N: number of patients;
p value of exposure metrics parameter estimate using Wald test;
Sign: Sign of exposure metrics parameter estimate in logistic regression (negative sign indicates that probability of response tends to decrease with exposure; positive sign indicates that response probability tends to increase with exposure;
NA: not applicable when not significant.)

To mitigate confounding between prognostic factors and atezolizumab clearance and exposure, a multivariate OS model was developed to account for baseline prognostic factors and TGI metrics as outlined. Median OS in OAK patients with NSCLC (n=388 TGI evaluable of 425 intention-to-treat [ITT]patients [91%]) was 467 days (95% CI, 402-508 days) and in IMvigor211 patients with UC (n=382 TGI evaluable of 467 ITT patients [82%]) was 344 days (95% CI, 290-383 days). Since median OS was shorter in mUC patients compared with NSCLC patients, tumor type was incorporated in the multivariate model. Of 770 TGI-evaluable patients, 764 had exposure data.

Individual estimates of Log(tumor growth rate [KG]) and baseline prognostic factors such as ECOG performance status >0, baseline tumor size, albumin level, lactate dehydrogenase, alkaline phosphatase, PD-L1 status, and tumor type were strong independent predictors of OS (Table 5). Of note, after accounting for baseline covariates in the final model, cycle 1 atezolizumab exposure (AUC, Cₘᵢₙ or Cₘₐₓ at Cycle 1) was no longer significant (p>0.01) when tested on the final model.

TABLE 5
Parameter estimates of final multivariate OS model in OAK and
IMvigor211 with mUC tumor type as a factor.

| Parameter | Estimate | SE | Z | P |
|---|---|---|---|---|
| Intercept | 2.946 | 0.3142 | 9.377 | 6.776e−21 |
| mUC tumor type | −0.1661 | 0.06302 | −2.636 | 0.008378 |
| Log(KG, week⁻¹) | −0.6185 | 0.03816 | −16.21 | 4.372e−59 |
| ECOG PS > 0 | −0.3406 | 0.06253 | −5.447 | 5.13e−08 |
| Albumin (g/L) | 0.02767 | 0.006164 | 4.489 | 7.169e−06 |
| Tumor burden (mm) | −0.002817 | 0.0006747 | −4.175 | 2.974e−05 |
| Lactate dehydrogenase (IU) | −0.0005352 | 0.0001895 | −2.824 | 0.004739 |
| IC2/3 (vs IC0/1) | 0.2535 | 0.08514 | 2.977 | 0.002908 |
| Alkaline phosphatase (IU) | −0.001383 | 0.0003242 | −4.265 | 1.998e−05 |
| Log(scale) | −0.3191 | 0.03497 | −9.125 | 7.183e−20 |

Survival time was analyzed in days
ECOG PS = Eastern Cooperative Oncology Group performance status.
IC = PD-L1 expression on tumor-infiltrating immune cells;
KG = tumor growth rate constant from tumor growth inhibition model;
mUC = metastatic urothelial carcinoma;
OS = overall survival;
P = Wald test P value;
Scale = standard deviation of log(OS);
SE = standard error of parameter estimate;
Z = Wald test statistic.

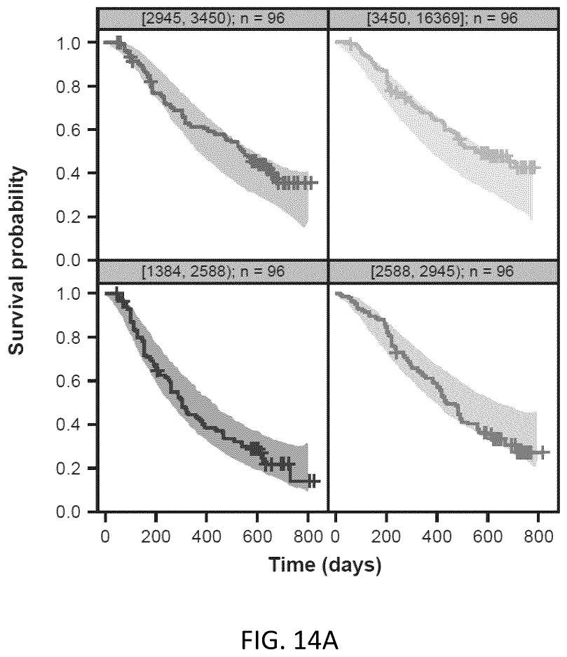

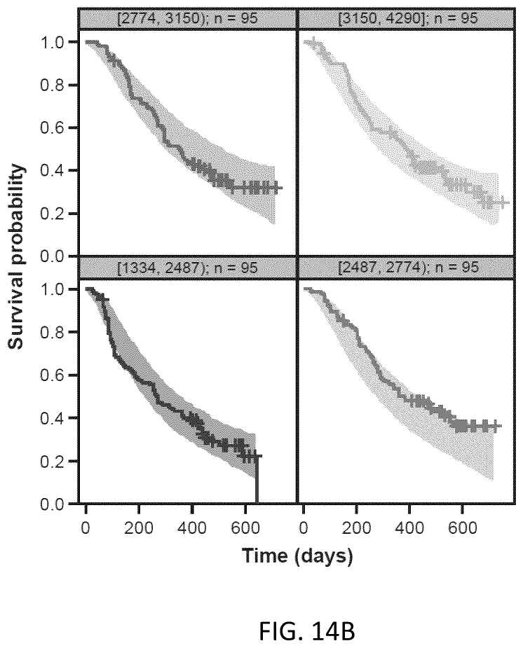

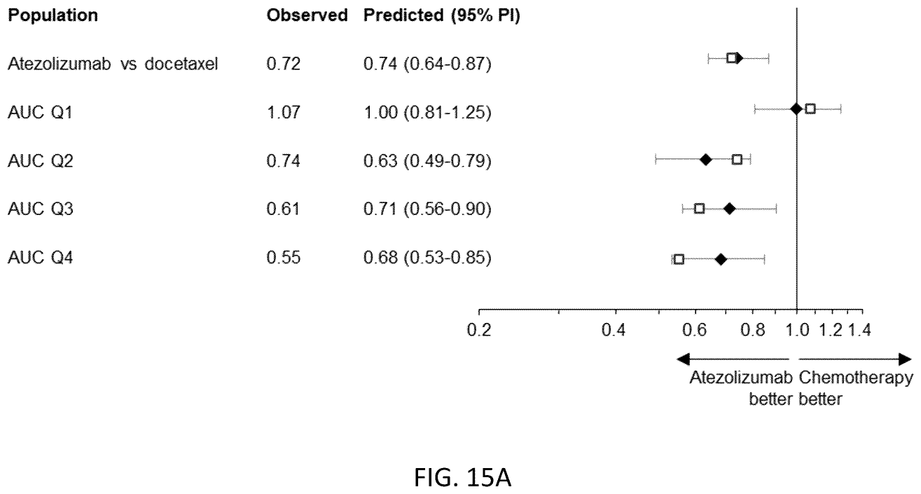

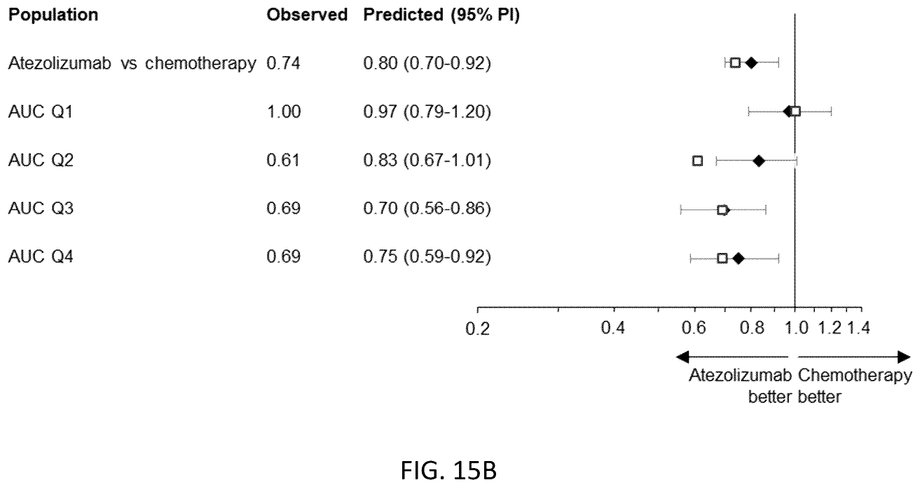

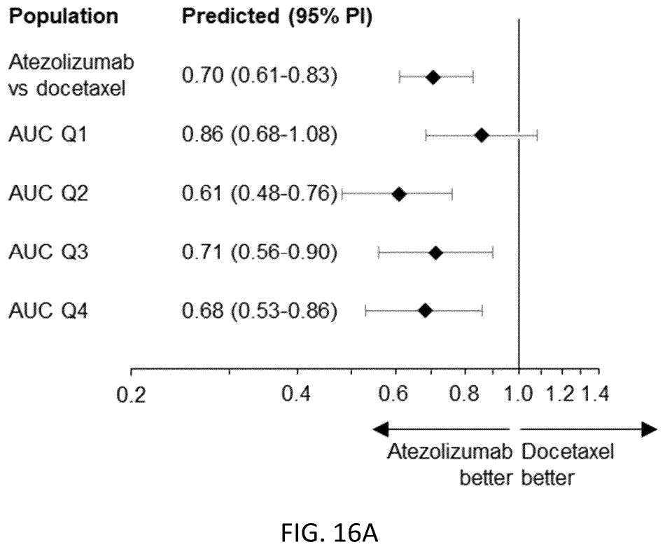

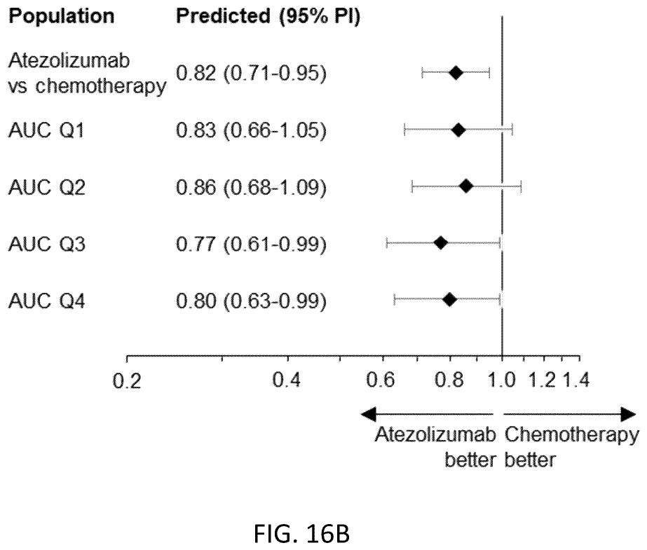

The model performed well in simulating OS distribution and HRs by exposure quartiles for each tumor type even if exposure was not in the model. Comparisons of predicted and observed OS data are provided in FIGS. 14A-14B and FIGS. 15A-15B. The flat ER relationship of atezolizumab was also illustrated in a simulation of the HRs by AUC quartiles after adjusting for baseline covariates (fixed to median values) FIGS. 16A-16B.

### Example 3

### Exposure-Safety Relationships for Atezolizumab in Urothelial Carcinoma and Non-Small Cell Lung Cancer

Exposure-safety analyses were conducted to assess possible relationships between safety endpoints and atezolizumab exposure for patient populations in each indication (UC or NSCLC) separately as well as pooled (UC and NSCLC).

### Methodology

### Urothelial Carcinoma

Adverse events of Grade 3 to 5 (AEG35) and adverse events of special interest (AESIs) from Study PCD4989g (UC cohort), IMvigor210 (Cohort 1 and Cohort 2), and IMvigor211 (atezolizumab arm) were analyzed for exposure-safety relationships. The safety endpoints were characterized by frequency (Yes/No). The proportions of frequency and 95% CI were computed for intervals of exposure with an equivalent number of individuals (e.g., quartiles). For each such correlation, a logistic regression was performed and the Wald test p-value for exposure effect in the logistic regression was reported.

### p(AE)˜Exposure

Where p(AE) is the probability of adverse event (i.e., AEG35 or AESI) and exposure is an atezolizumab exposure metric. Atezolizumab exposure metrics (AUC, Cₘₐₓ, and Cₘᵢₙ) were derived at Cycle 1 from simulated PK profiles based on individual PK parameters.

### Non-Small Cell Lung Cancer

The AEG35s and AESIs from the pooled data from studies BIRCH, POPLAR, FIR, and PCD4989g (NSCLC cohort), and OAK data separately, were used in the exposure-safety analyses. These safety endpoints were characterized by frequency (Yes/No). The proportions of frequency and 95% CI were computed for intervals of exposure with an equivalent number of individuals (e.g., quartiles). For each such correlation, a logistic regression was performed and the Wald test p-value for exposure effect in the logistic regression was reported.

### p(AE)˜Exposure

Where p(AE) is the probability of an adverse event (i.e., AEG35 or AESI) and exposure is an atezolizumab exposure metric. Atezolizumab exposure metrics (AUC, Cₘₐₓ, and Cₘᵢₙ) were derived at Cycle 1 from simulated PK profiles based on individual PK parameters.

### Pooled Analysis

Pooled analysis of Exposure-Safety relationships for atezolizumab in UC and NSCLC was carried out as described above and in the “Overview of pooled ER Analyses” section in Example 2.

Adverse events of Grade 2 to 5 (AEG25s), adverse events of Grade 3 to 5 (AEG35s), and adverse events of special interest (AESIs) in all atezolizumab treated mUC and NSCLC patients in Studies PCD4989g, IMvigor211, and OAK were analyzed for the relationship between exposure and safety. The safety endpoints were characterized by frequency (Yes/No). The proportions of frequency and 95% CI were computed for intervals of exposure with an equivalent number of individuals (e.g., quartiles). For each such correlation, a logistic regression was performed and the Wald test p-value for exposure effect in the logistic regression was reported.

### p(AE)˜Exposure

Where p(AE) is the probability of adverse event (i.e., AEG25, AEG35 or AESI) and exposure is an atezolizumab exposure metric. Atezolizumab exposure metrics (AUC, Cₘₐₓ, and Cₘᵢₙ) were derived at Cycle 1 from simulated PK profiles based on individual PK parameters.

### Results

### Urothelial Carcinoma

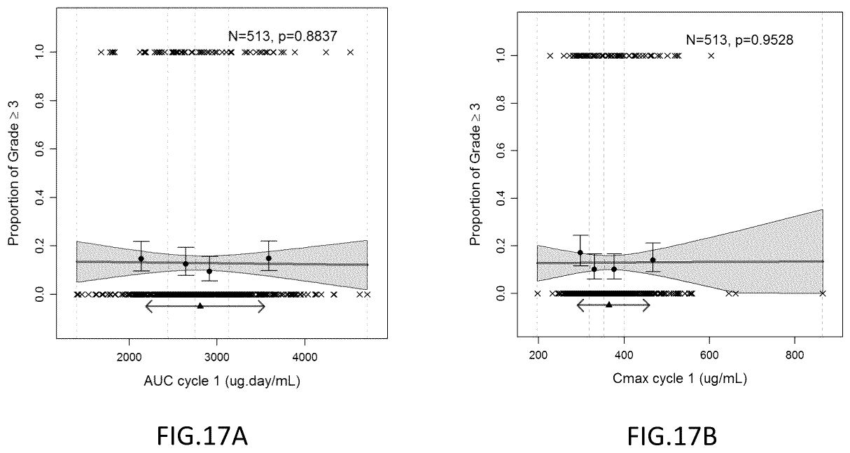

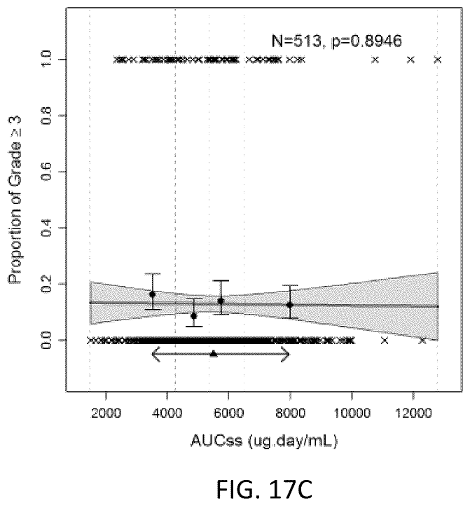

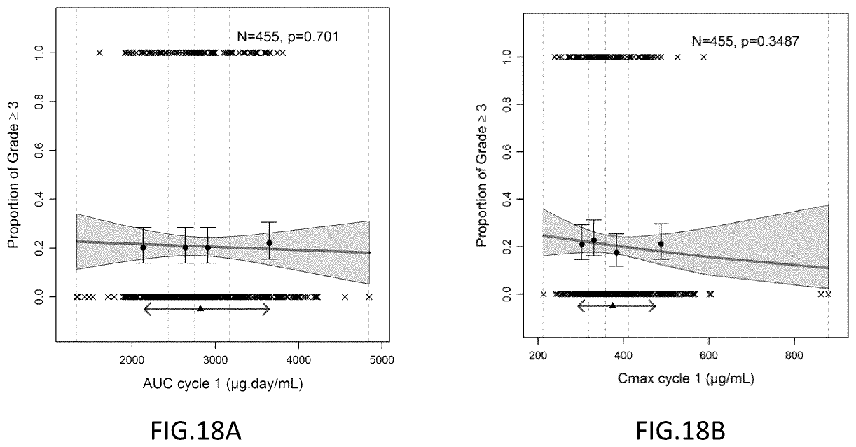

The analysis of the incidence of AEG35s did not show any statistically significant ER relationship with any exposure metric investigated, including Cycle 1 AUC (FIG. 17A), Cₘₐₓ(FIG. 17B), or AUCₛₛ (FIG. 17C) in a combined analysis of UC patients in PCD4989g and IMvigor210 or Cycle 1 AUC (FIG. 18A) or Cₘₐₓ FIG. 18B in an independent analysis of Study IMvigor211.

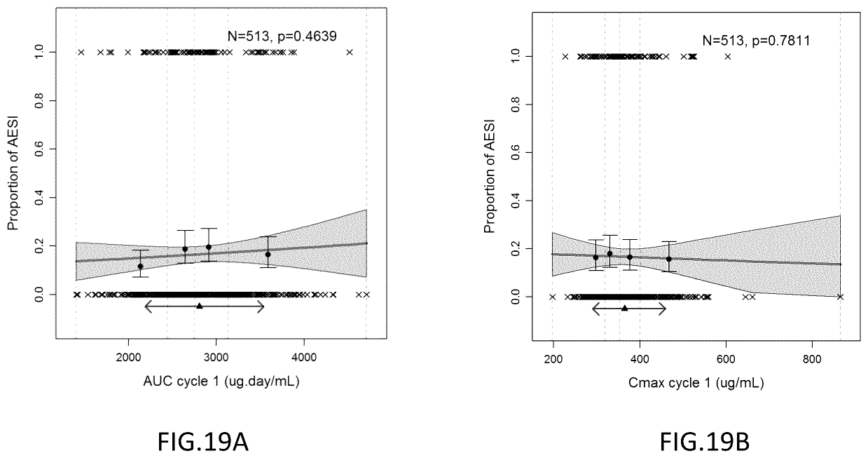

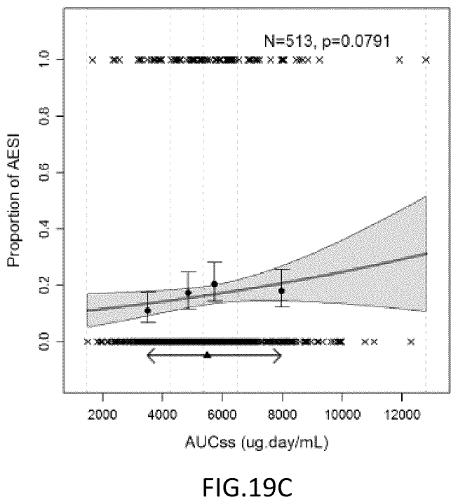

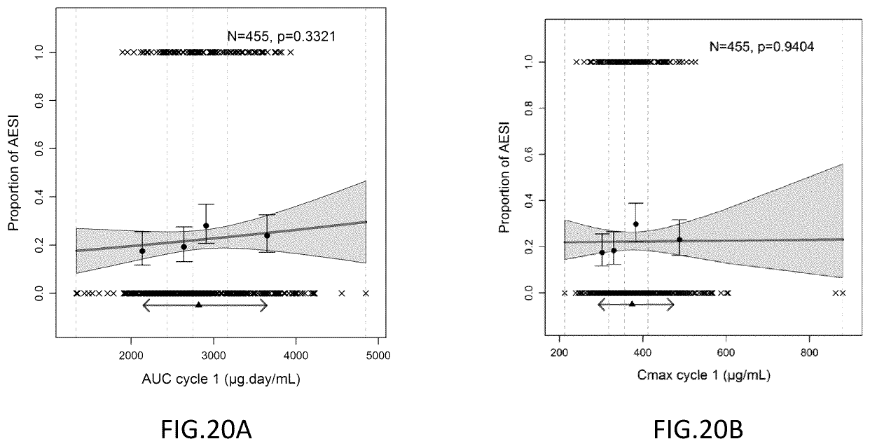

Similarly, the analysis of the incidence of AESIs did not show any statistically significant ER relationship with any exposure metric investigated, including Cycle 1 AUC (FIG. 19A), Cycle 1 Cₘₐₓ (FIG. 19B), or AUCₛₛ (FIG. 19C) in a combined analysis of UC patients in PCD4989g and IMvigor210, or Cycle 1 AUC (FIG. 20A) or Cycle 1 Cₘₐₓ (FIG. 20B) in an independent analysis of Study IMvigor211.

### Non-Small Cell Lung Cancer

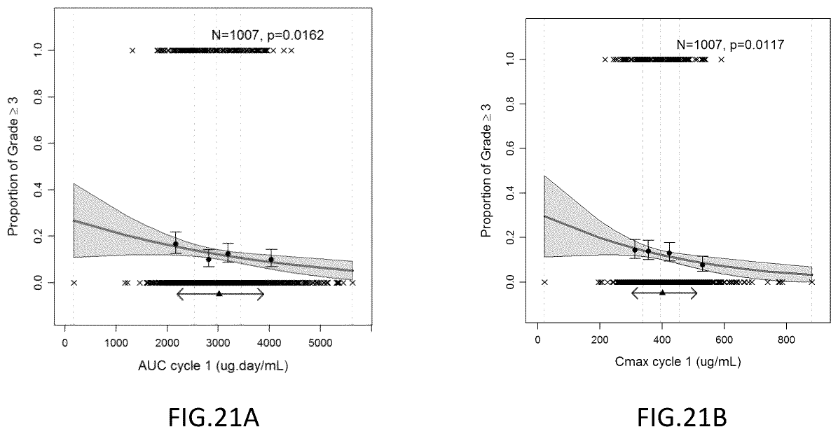

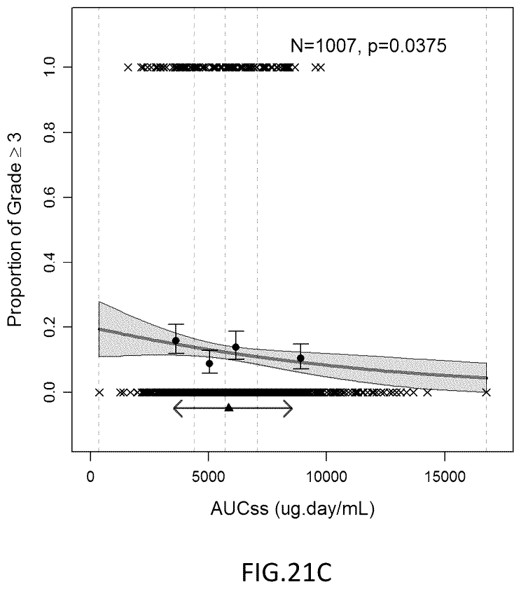

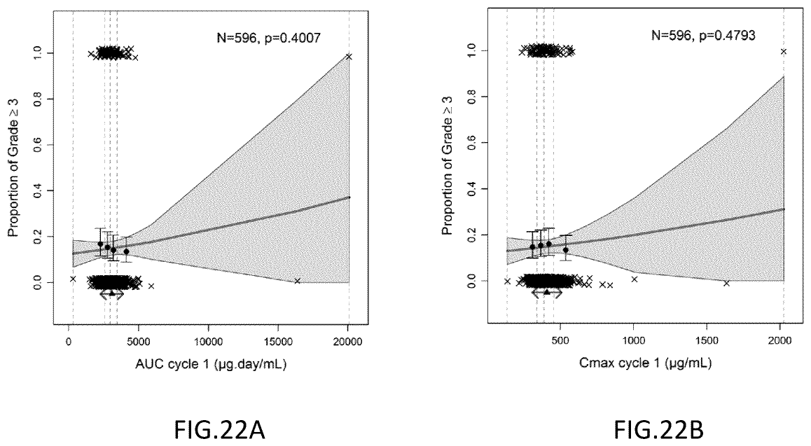

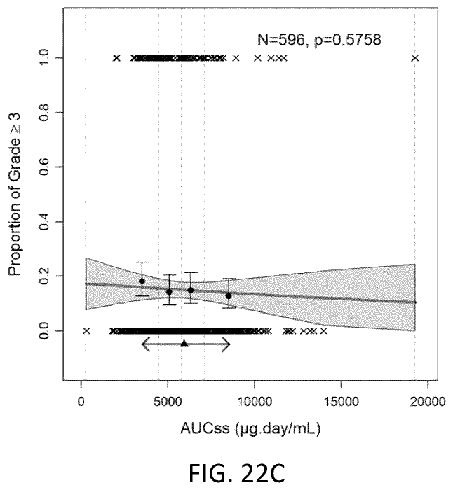

The analysis of the incidence of AEG35 did not show any statistically significant positive ER relationship with any exposure metric investigated, including Cycle 1 AUC (FIG. 21A), Cycle 1 Cₘₐₓ (FIG. 21B), and AUCₛₛ (FIG. 21C) in a combined analysis of NSCLC patients in PCD4989g, BIRCH, POPLAR, and FIR, or Cycle 1 AUC (FIG. 22A), Cycle 1 Cₘₐₓ (FIG. 22B) or AUCₛₛ (FIG. 22C) in an independent analysis of OAK.

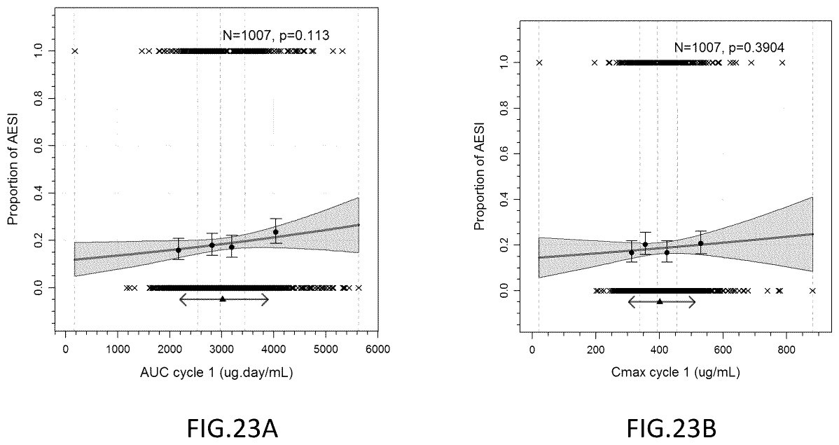

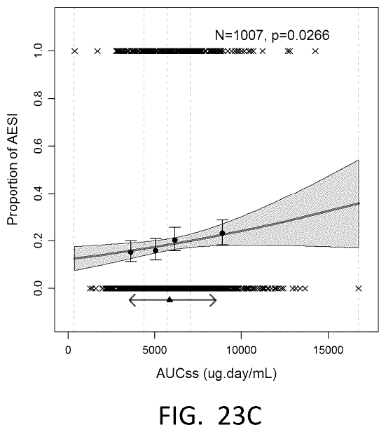

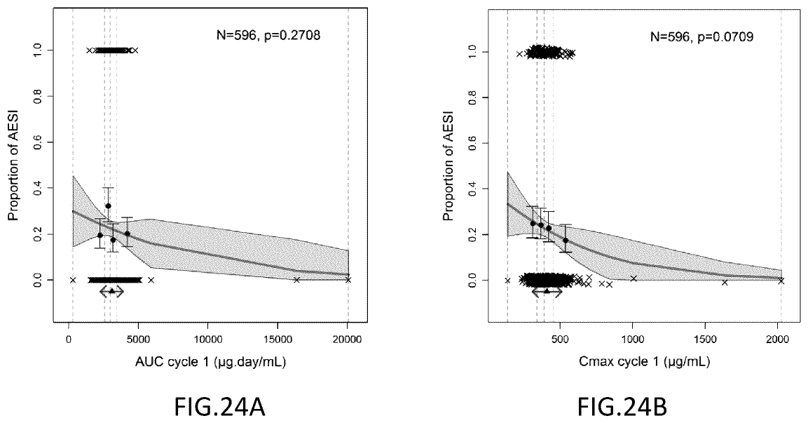

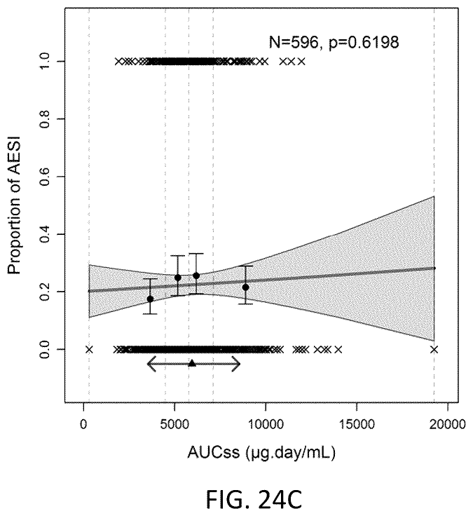

The analysis of the incidence of AESIs of the pooled analysis of NSCLC patients in PCD4989g, BIRCH, POPLAR, and FIR did not show any statistically significant ER relationship with Cycle 1 AUC (FIG. 23A), or Cₘₐₓ (FIG. 23B), but did have a statistically significant relationship with AUCₛₛ (FIG. 23C). For OAK, the analysis of the incidence of AESIs did not show any statistically significant ER relationship with any exposure metric investigated, including Cycle 1 AUC (FIG. 24A), Cycle 1 Cₘₐₓ (FIG. 24B) or AUCₛₛ (FIG. 24C).

For the pooled data from studies BIRCH, POPLAR, FIR, and PCD4989g (NSCLC cohort), the AESIs included a number of different events; the most frequent AESIs (seen in 15 patients or more) were evaluated for relationship to AUCₛₛ. While findings suggested a slight increase in the probability of AESI, this increase was not considered to be clinically meaningful or to require dose adjustment. This finding with regards to AESI was not observed in OAK. The reason for the discrepancy between the significance of the AESI atezolizumab ER for AUCₛₛ between OAK and the earlier pooled study data is not known. It should also be noted that as detailed below, the ER trend identified in the pooled study data for AESI is not regarded clinically meaningful.

For the pooled data from studies BIRCH, POPLAR, FIR, and PCD4989g (NSCLC cohort), simulation of the logistic regression model for AUCₛₛ suggests an increase in the probability of AESIs (estimate [prediction interval]) from 0.18 (0.16, 0.21) to 0.22 (0.18, 0.26) for patients with the median and 90^{th} percentile of AUCₛₛ, respectively. For the pooled study data, this increase in AESIs is not anticipated to be clinically meaningful or to require dose adjustment. Of the statistically significant covariates identified by the Phase I popPK Model, simulations suggested the largest positive estimated change in atezolizumab AUCₛₛ was >32% and was associated with the extreme values (i.e., 10% percentile) of weight. Since no single effect was associated with a >32% change in AUCₛₛ, none of the changes in AUCₛₛ associated with the statistically significant covariates identified with the popPK model would be expected to be clinically meaningful or to require dose adjustment. The elevation in AUCₛₛ when evaluated at extreme values (i.e., 10^{th} percentile) of weight compared with the typical patient following administration of the atezolizumab 1200 mg q3w flat dose would not be expected to be clinically meaningful or to require dose adjustment by BW.

### Pooled (NSCLC and UC) Analysis

Pooled atezolizumab exposure-safety analyses were performed on all atezolizumab-treated patients with locally advanced or metastatic NSCLC or UC with exposure data (n=1228).

AEs of grade ≥3 and AESIs occurred in 209 (17.0%) and 298 (24.3%) of 1228 patients, respectively. AE frequencies were similar in patients with NSCLC compared with UC (14.9% vs 19.6% for grade ≥3 AEs; 24.6% vs 23.9% for AESIs); therefore, tumor type was not included in the logistic regression models.

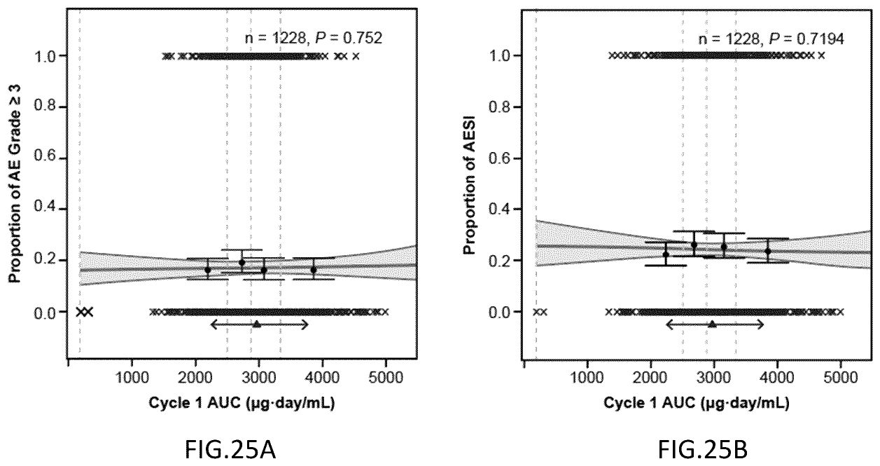

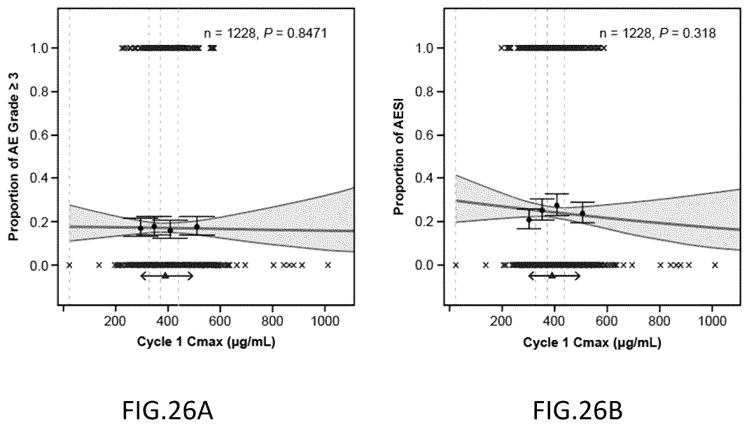

The analysis of the incidence of AEG35 (grade >3 AEs) in all atezolizumab treated mUC and NSCLC patients in Studies PCD4989g, IMvigor211, and OAK did not show any statistically significant ER relationship with any cycle 1 exposure metric investigated, including Cycle 1 AUC (FIG. 25A) or Cₘₐₓ (FIG. 26A).

Similarly, the analysis of the incidence of AESIs in all atezolizumab treated mUC and NSCLC patients in Studies PCD4989g, IMvigor211, and OAK did not show any statistically significant ER relationship with any cycle 1 exposure metric investigated, including Cycle 1 AUC (FIG. 25B) or Cₘₐₓ (FIG. 26B).

### Example 4

### Comparison of Observed Atezolizumab Exposure and Predicted 840-Mg q2w and 1680-Mg q4w Exposures

### Summary of Examples 1-3

As described above, for the approved 1200-mg q3w dosing regimen, atezolizumab exhibited ER trends that are not considered clinically meaningful or ER trends that are confounded by prognostic factors for both efficacy and safety in patients with metastatic UC or NSCLC. In terms of ER for efficacy for both UC and NSCLC, no clinically meaningful ER relationship with ORR or OS has been observed (see Example 2). This suggests that the exposure achieved by the approved 1200-mg q3w dosing regimen is in the flat or plateau part of the ER curve.

Therefore, no impact on response is expected as long as any new dosing regimen achieves exposure within the range that is expected for the approved 1200-mg q3w dosing regimen. Importantly, the 840-mg q2w and 1680-mg q4w dosing regimens are expected to fall within this exposure range.

In terms of ER for safety for both UC and NSCLC, no clinically meaningful ER for atezolizumab for safety has been observed for doses ranging from 10 mg/kg q3w to 20 mg/kg q3w, which includes the 1200-mg fixed dose q3w regimen (see Example 3). The fixed-dose regimens of 840 mg q2w, 1200 mg q3w, and 1680 mg q4w are equivalent to 10.5 mg/kg q2w, 15 mg/kg q3w, and 21 mg/kg q4w, respectively, when normalized for an 80 kg BW. Any new atezolizumab dosing regimen that provides exposure within the range observed for doses ranging up to 20 mg/kg q3w (the highest dose administered in the first-in-human dose-ranging Study PCD4989g, which was generally well tolerated) is expected to exhibit similar exposure-safety relationships to those previously observed. The 840-mg q2w and 1680-mg q4w dosing regimens are expected to fall within the exposure range observed for the approved 1200-mg q3w dosing regimen and 20 mg/kg q3w (see Example 6). It should be noted that a maximum tolerated dose (MTD) was not determined in the dose-ranging Study PCD4989g.

In this Example, PK profiles of virtual patients were predicted for 840 mg q2w, 1200 mg q3w, 1680 mg q4w, and 20 mg/kg q3w dosing regimens based on the popPK model described in the preceding Examples. Atezolizumab exposure metrics were then derived from the simulated PK profiles.

### Methodology

A population PK model of atezolizumab developed previously (see preceding Examples) was used to predict individual PK profiles in virtual patients at Cycle 1 and steady-state for the following dosing regimens: 840 mg q2w, 1200 mg q3w, 1680 mg q4w, and 20 mg/kg q3w.

Atezolizumab exposure metrics (Cₘₐₓ, C_{trough} and AUC at Cycle 1 and steady state) were derived from the simulated individual PK profiles, and summarized across individuals for each dosing regimen. In order to compare several dosing regimens involving different dosing intervals (every 2, 3 or 4 weeks), weekly AUC at Cycle 1 and steady-state were also derived. The difference in geometric mean of weekly AUC,ₛₛ for each dosing regimen to weekly AUC,ₛₛof 20-mg/kg q3w (the highest dose administered in the first-in-human dose-ranging Study PCD4989g) was calculated.

To simulate PK parameters of varying regimens of atezolizumab (840 mg q2w, 1200 mg q3w, 1680 mg every 4 weeks [q4w], and 20 mg/kg q3w), Monte Carlo simulations were performed using the popPK model of atezolizumab, including covariate effects, previously developed using PCD4989g data (Stroh et al., (2017) Clin Pharmacol Ther doi: 10.1002/cpt.587) to obtain virtual individual PK profiles at cycle 1 and steady state. In the popPK model used for the PK simulations, bodyweight, albumin, tumor burden, treatment-emergent antidrug antibody (ADA) status, and gender were found to have a statistically significant impact on atezolizumab PK. A single replicate of 500 patients was simulated for each regimen. A seed number was provided in the control stream to ensure reproducibility of the simulations. Random effects were sampled from the previously estimated distribution, and the residual error was not taken into account for individual predictions. Virtual patients per dosing regimen were assumed to have a 1:1 male:female ratio (males weighing 85 kg and females weighing 64 kg, median body weight in the phase 1 database used to develop the popPK model). Other covariates affecting atezolizumab PK parameters were set to the median or most frequent category for the categorical covariates: albumin level of 40 g/L, baseline tumor size of 63 mm, and negative for antidrug antibodies (ADAs). Four dosing regimens were simulated: 1200 mg q3w, 20 mg/kg q3w (i.e., 1700 mg for males and 1280 mg for females), 840 mg q2w, and 1680 mg q4w. In order to assess the impact of body weight on exposure after the fixed-dose regimen, 500 virtual patients per quartile of body weight with median albumin level, baseline tumor size, and negative for ADAs were assigned a dose of 840 mg q2w or 1680 mg q4w. The distribution of body weight in the phase 1 population of patients was divided by quartiles as follows: 36.5 to 63.7, 63.7 to 77.0, 77.0 to 90.9, and 90.9 to 168.0 kg. The 500 individual body weights were sampled in each quartile assuming a truncated normal distribution. In order to maintain the correlation between sex and body weight, the proportion of females was set to 80% in the first quartile, 50% in the second quartile, 25% in the third quartile, and 10% in the last quartile, as observed in the phase 1 database used to develop the popPK model.

Atezolizumab exposure metrics (cycle 1: AUC [calculated using the trapezoidal method; time 0-21 days], Cₘₐₓ, and Cₘᵢₙ; steady state: AUC [dose/clearance], Cₘₐₓ, and Cₘᵢₙ) were derived from the simulated individual PK profiles and summarized across individuals for each dosing regimen. In order to compare several dosing regimens involving different dosing intervals (every 2, 3, or 4 weeks), steady-state weekly AUC data were also derived.

### Results

Population PK-simulated exposures for regimens of 840 mg every 2 weeks (q2w) and 1680 mg every 4 weeks (q4w) were compared with the approved regimen of 1200 mg every 3 weeks (q3w) and the maximum assessed dose (MAD; 20 mg/kg q3w).

A summary of popPK estimated exposures from all available studies for Cycle 1 and at steady-state is provided in Table 5B and Table 6 below, respectively.

TABLE 5B
Summary Statistics (Geometric Mean, % CV) of 1200 mg q3w Atezolizumab
Exposure Metrics in Cycle 1 Predicted using PopPK Model (PK-Evaluable Population).

| | | No. of | Cₘₐₓ | Cₘᵢₙ | AUC |
|---|---|---|---|---|---|
| Study | Dose Level | Patients | [μg/mL] | [μg/mL] | [μg · day/mL] |
| PCD4989g | 10 mg/kg | 35 | 259 [14.1] | 41.6 [16.2] | 2072 [13.5] |
| | 15 mg/kg | 233 | 360 [19.8] | 53.6 [29.4] | 2717 [23.8] |
| | 20 mg/kg | 147 | 488 [19.5] | 75.0 [23.5] | 3749 [22.2] |
| | 1200 mg^{a} | 45 | 432 [19.1] | 87.4 [27.2] | 3334 [19.9] |
| JO28944 | 10 mg/kg | 3 | 207 [8.4] | 32.1 [8.6] | 1548 [2.9] |
| | 20 mg/kg | 3 | 509 [5.4] | 82.7 [9.6] | 4068 [4.1] |
| IMvigor210 | 1200 mg | 117 | 370 [17.8] | 71.1 [32.9] | 2850 [18.8] |
| | [Cohort 1] | | | | |
| | 1200 mg | 306 | 355 [17.8] | 69.1 [28.9] | 2728 [19.1] |
| | [Cohort 2] | | | | |
| IMvigor211 | 1200 mg^{a} | 455 | 367 [19.5] | 64.6 [49.5] | 2762 [20.4] |
| BIRCH | 1200 mg^{a} | 652 | 402 [20.6] | 77.6 [34.9] | 3039 [22.0] |
| FIR | 1200 mg^{a} | 128 | 391 [19.6] | 68.9 [44.4] | 2855 [23.1] |
| POPLAR | 1200 mg^{a} | 140 | 355 [17.9] | 63.1 [34.0] | 2599 [20.5] |
| OAK | 1200 mg^{a} | 596 | 396 [22.8] | 74.6 [43.3] | 2978 [26.1] |

AUC = area under the concentration-time curve;
Cₘₐₓ = maximum serum concentration;
Cₘᵢₙ = trough or minimum serum concentration;
% CV = percent coefficient of variation;
PK = pharmacokinetic.
^{a}1200 mg equivalent to 15 mg/kg (80-kg patient).

TABLE 6
Summary Statistics (Geometric Mean, % CV) of 1200 mg q3w Atezolizumab Exposure
Metrics at Steady-State using PopPK Model (PK-Evaluable Population).

| | | No. of | C_{max,ss} | C_{min,ss} | AUCₛₛ |
|---|---|---|---|---|---|
| Study | Dose Level | Patients | [μg/mL] | [μg/mL] | [μg · day/mL] |
| PCD4989g | 10 mg/kg | 35 | 384 [16.0] | 120 [33.8] | 3993 [23.6] |
| | 15 mg/kg | 233 | 522 [25.0] | 148 [62.5] | 5141 [40.7] |
| | 20 mg/kg | 147 | 715 [21.7] | 213 [48.5] | 7206 [32.9] |
| | 1200 mg^{a} | 45 | 634 [24.0] | 193 [45.7] | 6409 [33.7] |
| J028944 | 10 mg/kg | 3 | 307 [4.5] | 97.3 [22.6] | 3114 [13.6] |
| | 20 mg/kg | 3 | 799 [9.5] | 288 [17.0] | 8787 [12.1] |
| IMvigor210 | 1200 mg^{a} | 117 | 544 [22.3] | 165 [48.4] | 5528 [33.2] |
| | [Cohort 1] | | | | |
| | 1200 mg^{a} | | | | |
| | [Cohort 2] | 306 | 513 [22.5] | 150 [47.3] | 5133 [32.9] |
| IMvigor211 | 1200 mg^{a} | 455 | 520 [22.6] | 142 [53.9] | 5018 [34.0] |
| BIRCH | 1200 mg^{a} | 652 | 582 [24.9] | 170 [51.8] | 5770 [35.4] |
| FIR | 1200 mg^{a} | 128 | 550 [25.8] | 145 [64.7] | 5199 [41.3] |
| POPLAR | 1200 mg^{a} | 140 | 492 [22.7] | 129 [54.6] | 4636 [35.4] |
| OAK | 1200 mg^{a} | 596 | 570 [27.9] | 162 [61.2] | 5573 [38.7] |

AUCₛₛ = area under the concentration-time curve at steady state;
C_{max,ss} = maximum serum concentration at steady state;
C_{min,ss} = trough or minimum serum concentration at steady state;
CV = coefficient of variation;
PK = pharmacokinetic;
q3w = every 3 weeks.
^{a}1200 mg equivalent to 15 mg/kg (80-kg patient).

PopPK predicted simulated atezolizumab exposure profiles (concentration-time profiles) of 4 dosing regimens (840-mg q2w, 1200-mg q3w, 1680-mg q4w, and 20-mg/kg q3w) are presented in FIG. 27. The profiles are displayed over a 28-day period showing 2 doses for 1200-mg q3w, 20-mg/kg q3w and 840-mg q2w; and 1 dose for 1680-mg q4w. A summary of the corresponding exposure metrics associated with each dosing regimen (predicted Cₘₐₓ and Cₘᵢₙ values at Cycle 1 and at steady state) is presented in Table 7.

TABLE 7
Summary Statistics (Geometric Mean [90% CI] for 500 patients)
for Atezolizumab Exposure Simulated for Various Regimens.

| | Cₘₐₓ (μg/mL) | | Cₘᵢₙ (μg/mL) | |
|---|---|---|---|---|
| Regimen | Cycle 1 | Steady-State | Cycle 1 | Steady-State |
| 1200-mg q3w | 403 [274, 581] | 610 [414, 891] | 85 [55, 133] | 194 [89, 383] |
| 840-mg q2w | 281 [187, 420] | 517 [334, 801] | 74 [48, 116] | 226 [118, 426] |
| 1680-mg q4w | 563 [379, 822] | 759 [514, 1106] | 97 [58, 159] | 182 [87, 369] |
| 20-mg/kg q3w | 501 [378, 665] | 753 [544, 1038] | 107 [70, 149] | 238 [115, 443] |

Cₘₐₓ: maximum serum atezolizumab concentration;
Cₘᵢₙ: minimum serum atezolizumab concentration;
q2w: every 2 weeks;
q3w: every 3 weeks;
q4w: every 4 weeks

The predicted weekly Cycle 1 AUC and AUCₛₛ are presented in Table 8.

TABLE 8
Summary Statistics (Geometric Mean [90% CI] for 500 patients)
for Atezolizumab Exposure Simulated for Various Regimens.

| | Weekly AUC (μg · day/mL)^{a} | | |
|---|---|---|---|
| | | | Difference |
| | | | at SS from |
| | | | 20-mg/kg |
| Regimen | Cycle 1 | Steady-State | q3w (%) |
| 1200-mg q3w | 1048 [763, 1471] | 2115 [1264, 3507] | −18.5 |
| 840-mg q2w | 860 [617, 1237] | 2188 [1336, 3733] | −15.7 |
| 1680-mg q4w | 1288 [887, 1845] | 2217 [1357, 3705] | −14.6 |
| 20-mg/kg q3w | 1305 [1002, 1683] | 2596 [1592, 4140] | — |

AUC: area under the concentration-time curve;
SS: steady-state;
q2w: every 2 weeks;
q3w: every 3 weeks;
q4w : every 4 weeks
^{a}Weekly AUC over 3 weeks (for q3w regimen), over 4 weeks (for q4w regimen) and over 2 weeks (for q2w regimen)

The 840-mg q2w dosing regimen has a predicted Cₘᵢₙ concentration that is 13% lower at Cycle 1 and 16% higher at steady-state than the predicted Cₘᵢₙ of the 1200-mg q3w dosing regimen. However, the predicted Cₘᵢₙ values for the 840-mg q2w regimen at Cycle 1 and steady-state are still at least 10-fold greater (>10 fold) than the Cₘᵢₙ target concentration (6 g/mL (Deng et al., (2016) MAbs doi: 10.1080/19420862.2015.1136043)). The predicted Cₘₐₓ of the 840-mg q2w dosing regimen is lower than the predicted Cₘₐₓ of the 1200-mg q3w dosing regimen at Cycle 1 and steady-state.

The 1680-mg q4w dosing regimen (equivalent to a 21-mg/kg q4w dose for an 80-kg patient) has a predicted Cₘᵢₙ that is 14% higher at Cycle 1 and 6% lower at steady-state than the predicted Cₘᵢₙ of the 1200-mg q3w dosing regimen. However, the predicted Cₘᵢₙ values for the 1680-mg q4w regimen at Cycle 1 and steady-state are still at least 10-fold greater (>10 fold) than the Cₘᵢₙ target concentration (6 g/mL).

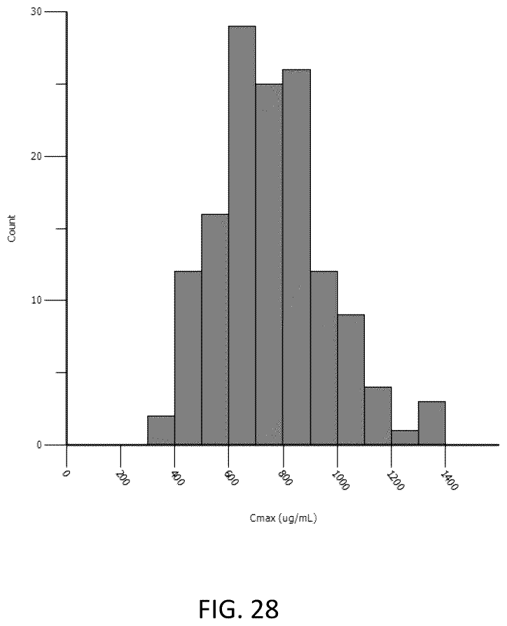

The predicted Cₘₐₓ of the 1680-mg q4w regimen is 12% higher at Cycle 1 and 0.8% higher at steady-state, respectively, relative to the predicted geometric mean Cₘₐₓ for the 20-mg/kg dosing regimen, and was consistent with observed exposures for the 20-mg/kg q3w dosing regimen in PCD4989g (Stroh et al., (2017) Clin Pharmacol Ther doi: 10.1002/cpt.587; Center for Drug Evaluation and Research (2016) BLA 761034 Clinical Pharmacology Review—Atezolizumab, available at the website www[dot]accessdata[dot]fda[dot]gov/drugsatfda_docs/nda/2016/761034Orig1s000ClinPharmR.pdf). The predicted 90^{th} percentiles of Cₘₐₓ for the 1680-mg q4w regimen at Cycle 1 and steady-state are 754 μg/mL and 1037 g/mL, respectively. Despite this tendency toward a higher Cₘₐₓ at Cycle 1 than the 20-mg/kg dosing regimen, the 1680-mg q4w dosing regimen predicted exposure is still within the range of the exposure observed for the 20-mg/kg q3w dosing regimen in Study PCD4989g (FIG. 28).

The predicted weekly AUC for the regimens of 840 mg q2w and 1680 mg q4w at steady state were higher than those simulated for 1200 mg q3w by 3.5% and 4.8%, respectively.

When considering fixed-dose regimens, since clearance and volume are impacted by body weight in the atezolizumab popPK model (Stroh et al., (2017) Clin Pharmacol Ther doi: 10.1002/cpt.587), patients with lower body weight would be expected to exhibit higher atezolizumab exposure relative to heavier patients. To further evaluate the q2w and q4w regimens, Cₘᵢₙ or Cₘₐₓ were simulated by quartiles of body weight for dose levels of 840 mg q2w and 1680 mg q4w (Table 9).

For the 1680-mg q4w regimen, the predicted Cₘₐₓ values for the lowest body weight quartile (<63.7 kg, with a majority of females) were 692 and 950 μg/mL for cycle 1 and steady state, respectively, which is within the range of the observed Cₘₐₓ values for 1200 mg q3w and 20 mg/kg q3w (Stroh et al., (2017) Clin Pharmacol Ther doi: 10.1002/cpt.587; Center for Drug Evaluation and Research (2016) BLA 761034 Clinical Pharmacology Review—Atezolizumab, available at the website www[dot]accessdata[dot]fda[dot]gov/drugsatfda_docs/nda/2016/761034Orig1s000ClinPharmR.pdf). For the 840-mg q2w regimen, the predicted Cₘᵢₙ values for the highest body weight quartile (>90.9 kg, with a majority of males) were 58 and 158 μg/mL for cycle 1 and steady state, respectively, which is within the range of the observed Cₘᵢₙ values for 1200 mg q3w and above the Cₘᵢₙ target concentration of 6 g/mL.

As noted above, the predicted Cₘₐₓ values of patients with the lowest bodyweight taking the 1680-mg q4w regimen are within range of the observed Cₘₐₓ values of the 20-mg/kg q3w dosing regimen in Study PCD4989g (FIG. 28).

TABLE 9
Simulated atezolizumab Cmax and Cmin values by body weight quartile.

| | | Body weight quartile, kg^{a} | | | | | | | |
|---|---|---|---|---|---|---|---|---|---|
| | | [36.5, 63.7) | | [63.7, 77.0) | | [77.0, 90.9) | | [90.9, 168.0] | |
| 840 mg | Cₘᵢₙ (90% PI), | | | | | | | | |
| q2w | μg/mL | | | | | | | | |
| | Cycle 1 | 93 | (64-136) | 77 | (54-110) | 67 | (45-98) | 58 | (40-84) |
| | Steady state | 299 | (165-549) | 241 | (132-426) | 197 | (103-366) | 158 | (78-296) |
| 1680 mg | Cₘₐₓ (90% PI), | | | | | | | | |
| q4w | μg/mL | | | | | | | | |
| | Cycle 1 | 692 | (505-950) | 573 | (407-784) | 506 | (368-675) | 425 | (313-586) |
| | Steady state | 950 | (692-1325) | 781 | (564; 1052) | 683 | (499-939) | 562 | (405-777) |

Geometric means with 90% PIs (for 500 patients) are shown.
Cₘₐₓ = maximum serum atezolizumab concentration,
Cₘᵢₙ = minimum (trough) serum atezolizumab concentration,
PI = prediction interval,
q2w = every 2 weeks,
q4w = every 4 weeks.
^{a}For interval notation format [a, b), a is included, and b is excluded, such that a ≤ x < b

In summary, the 1680-mg q4w and the 840-mg q2w regimens are expected to have comparable efficacy (e.g., ORR and OS) and safety with the approved 1200 mg q3w regimen. Since the predicted exposures (Cₘᵢₙ) of the 840-mg q2w and 1680-mg q4w regimens exceed the target concentration (6 g/mL) and are within range of Cₘᵢₙ values of the approved 1200-mg q3w regimen, and there is no clinically meaningful ER relationship of atezolizumab exposure with ORR or OS in NSCLC or UC patients dosed with 1200-mg q3w (see Example 2), no impact on response is expected with the use of either 840-mg q2w or 1680-mg q4w regimens compared with the approved 1200-mg q3w regimen.

Similarly, since the predicted Cₘₐₓ value for the 840-mg q2w and 1680-mg q4w regimens are within range of Cₘₐₓ values of the maximum assessed dose of 20-mg/kg which was generally well tolerated and there is no clinically meaningful ER relationship of atezolizumab exposure with AEs grade >3 or AESIs in NSCLC or UC patients dosed with 1200-mg q3w or 20-mg/kg (see Example 3), the 840-mg q2w and 1680-mg q4w regimens are anticipated to have a safety profile similar to the approved 1200-mg q3w regimen. This is further supported by a detailed assessment of the safety profile in: (1) patients receiving 20 mg/kg q3w vs 1200 mg q3w dosing regimens, (2) patients with low BW, (3) patients with a Cₘₐₓ above the predicted 90^{th} percentile of the 1680-mg q4w regimen (4) patients with a Cₘₐₓ above the predicted mean of 1680-mg q4w (see Examples 6-9).

### Example 5

Validation of popPK-Predicted 840-Mg q2w Exposure in TNBC

In this Example, Phase 3 IMpassion130 (NCT02425891) data were used to validate the PK simulations for 840 mg q2w.

### Materials and Methods

A prediction-corrected visual predictive check (pcVPC) was performed based on the prior phase 1 popPK model (external evaluation). The phase 1 popPK model was used to derive the individual PK parameter estimates based on atezolizumab observed concentration-time profiles in IMpassion130. PK data for atezolizumab-treated patients in IMpassion130 were simulated (1000 replicates) using actual dosing and patient covariates (body weight, sex, ADA status, albumin level, and tumor burden) and the phase 1 popPK model. Observed atezolizumab peak (Cₘ) and trough (Cₘᵢₙ) concentrations in IMpassion130 were compared with corresponding predictive distributions.

### Results

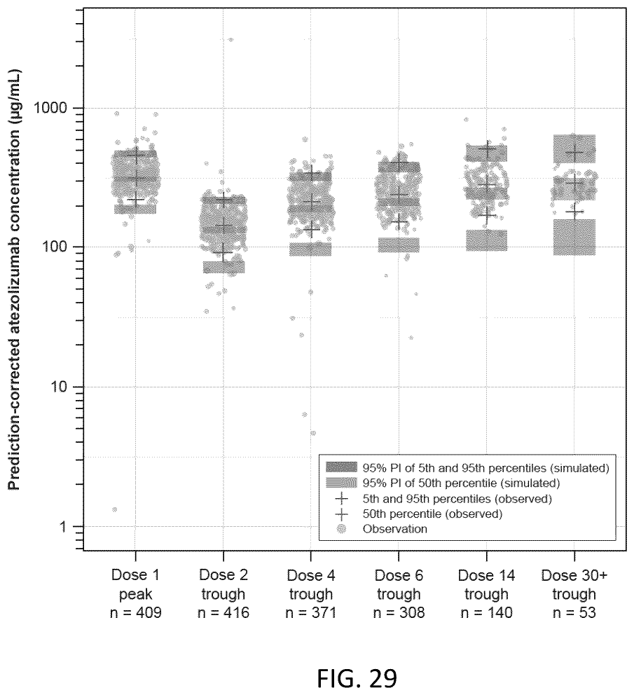

As an external evaluation of the phase 1 popPK model and to confirm the 840-mg q2w PK simulations, the PK of the atezolizumab plus nab-paclitaxel q2w arm from the IMpassion130 study were simulated based on baseline patient covariates (pcVPC). Four-hundred forty-three (of 445) atezolizumab-treated patients had evaluable serum samples for PK analysis, for a total of 2232 samples. Results are presented in FIG. 29. Both dose 1 and steady-state exposure metrics were similar to those predicted for the 840-mg q2w dosing regimen based on the phase 1 popPK model. A trend toward underprediction of the median and fifth percentile of atezolizumab exposure data (troughs) after longer-term administration (doses 2, 4, 6, 14, and 30+) was observed for the popPK model, consistent with the time-dependent clearance of atezolizumab (Tecentriq (atezolizumab) [package insert]. South San Francisco, CA: Genentech, Inc.; 2019. South San Francisco, CA, USA: Genentech, Inc).

### Example 6

Summary of Clinical Safety Data from Study PCD4989g, Including 20 mg/Kg q3w (Highest Dose Tested in Study PCD4989g)

The 20-mg/kg q3w dose provides a range of clinical exposure similar to the predicted steady-state maximum or Cₘₐₓ concentration of 759 μg/mL for the 1680-mg q4w fixed dose dosing regimen. No dose-limiting toxicities were observed at the 20-mg/kg dose level, and the incidence and intensity of AEs reported have not been shown to be dependent on dose. Thus, a maximum tolerated dose has not been established.

In this Example, the safety of atezolizumab in Study PCD4989g is analyzed.

Analysis of Adverse Events in Patients with Cₘₐₓ Higher or Lower than the Predicted Cₘₐₓ for 1680 mg Dose

Of the 640 safety-evaluable patients from Study PCD4989g, 82 patients were identified as having an observed Cₘₐₓ at any time that was higher than 759 μg/mL; 62 of these patients were from the 20-mg/kg dose cohort. The observed safety for this group of 82 patients was then compared with the remaining 558 patients with observed Cₘₐₓ≤759 μg/mL in Study PCD4989g (Table 10).

TABLE 10
Overall Safety Profile of Patients in Study PCD4989g.

| | Cₘₐₓ > 759 μg/mL | | Cₘₐₓ ≤ 759 μg/mL | | All Patients | |
|---|---|---|---|---|---|---|
| Parameter | (N = 82) | | (N = 558) | | (N = 640) | |
| Any AE | 81 | (98.8%) | 546 | (97.8%) | 627 | (98.0%) |
| Related AE | 62 | (75.6%) | 389 | (69.7%) | 451 | (70.5%) |
| Grade 3-4 AE | 31 | (37.8%) | 289 | (51.8%) | 320 | (50.0%) |
| Related Grade | 12 | (14.6%) | 78 | (14.0%) | 90 | (14.1%) |
| 3-4 AE | | | | | | |
| Grade 5 AE | 0 | (0.0%) | 10 | (1.8%) | 10 | (1.6%) |
| Related Grade | 0 | (0.0%) | 3 | (0.5%) | 3 | (0.5%) |
| 5 AE | | | | | | |
| SAE | 29 | (35.4%) | 240 | (43.0%) | 269 | (42.0%) |
| Related SAE | 9 | (11.0%) | 50 | (9.0%) | 59 | (9.2%) |
| AE leading | 2 | (2.4%) | 28 | (5.0%) | 30 | (4.7%) |
| to drug dis- | | | | | | |
| continuation | | | | | | |

AE = adverse event;
Cₘₐₓ = maximum concentration observed;
SAE = serious adverse event.

Overall, in Study PCD4989g, the safety profiles of the 82 patients with observed Cₘₐₓ>759 μg/mL and the 558 patients with observed Cₘₐₓ≤759 μg/mL appear comparable and consistent with the known risks of atezolizumab monotherapy or the baseline diseases.

For example, of the common AEs (20% of patients), the majority were similar in patients with Cₘₐₓ>759 μg/mL and patients with Cₘₐₓ≤759 μg/mL, which included fatigue, pyrexia, nausea, diarrhea, constipation, dyspnoea, and decreased appetite. AEs reported by a higher proportion in patients with Cₘₐₓ>759 μg/mL and patients with Cₘₐₓ≤759 μg/mL (≥5% difference) were fatigue, chills, influenza-like illness, nausea, cough, dyspnea, productive cough, hemoptysis, pneumonitis, musculoskeletal pain, decreased appetite, dry skin, upper respiratory tract infection, and sinusitis. The severity of these events were mostly Grade 1 or 2, except for one instance of nausea and five instances of dyspnoea, which were reported as Grade 3 or 4. These events were considered expected to occur with either the study treatment or the underlying disease.

Patients with Cₘₐₓ>759 μg/mL experienced more study treatment-related AEs as assessed by investigators than patients with Cₘₐₓ≤759 μg/mL (75.6% vs. 69.7%). The majority of the most common of the treatment-related AEs (10% of patients) was similar in patients with Cₘₐₓ>759 μg/mL and patients with Cₘₐₓ≤759 μg/mL.

Analysis of Serious Adverse Events in Patients with Cₘₐₓ Higher or Lower than the Predicted Cₘₐₓ for 1680 mg Dose

The proportion of patients experiencing serious AEs (SAEs) was higher in patients with Cₘₐₓ≤759 μg/mL (43.0%) than in patients with Cₘₐₓ>759 μg/mL (35.4%), and Grade 3-4 SAEs were also higher in patients with Cₘₐₓ≤759 μg/mL (33.7%) than in patients with Cₘₐₓ>759 μg/mL (25.6%). The common SAEs (>2% of patients) reported in both subgroups included dyspnoea (2.4% vs. 3.9%) and pyrexia (3.7% vs. 2.9%). Infections and gastrointestinal disorders occurred more frequently in the Cₘₐₓ≤759 μg/mL subgroup than the Cₘₐₓ>759 g/mL subgroup, however, no individual preferred terms (PTs) were identified to account for the noted difference.

There were no fatal AEs in patients with Cₘₐₓ>759 μg/mL; there were 10 fatal AEs (1.7%) in patients with Cₘₐₓ≤759 μg/mL. The 10 fatal events included the following: respiratory failure, pneumonia, pulmonary hypertension, sepsis, head injury, overdose (alcohol and morphine), acute myocardial infarction, hepatic failure, hepatic hematoma, and death (unknown cause).

Of patients with Cₘₐₓ>759 μg/mL, 2 (2.4%) patients reported AEs that led to study drug withdrawal, which was lower than the frequency reported in patients with Cₘₐₓ≤759 μg/mL (28, 5.0%). The two AEs that led to study drug withdrawal in the Cₘₐₓ>759 μg/mL patient group were blood bilirubin increased and colitis, which are known AEs for atezolizumab.

Based on this safety data analysis from patients with observed Cₘₐₓ>759 μg/mL, atezolizumab at a dose of 1680 mg q4w is expected to be well tolerated with a manageable safety profile.

### Example 7

Comparison of Safety Analyses Based on the Atezolizumab Treatment Groups from Studies PCD4989g, IMvigor211, and OAK

### Methodology

### Analysis Populations

The safety population within this analysis included patients from studies PCD4989g, IMvigor211, and OAK who received at least one dose of atezolizumab, with patients assigned to treatment groups according to the actual treatment received. The following treatment groups and subgroups were used for safety analyses:

Study PCD4989g:
“PCD4989g 20 mg/kg” (N=146): Patients in Study PCD4989g who received atezolizumab doses of 20 mg/kg IV q3w.
“PCD4989g 1200 mg” (N=210): Patients in Study PCD4989g who received atezolizumab doses of 1200 mg IV q3w.

Study PCD4989g subgroups by BW:
“Lowest Quartile BW PCD4989g 20 mg/kg” (N=37): Patients in study PCD4989g dosed with 20 mg/kg atezolizumab who had a BW in the lowest quartile of the BW distribution in that cohort.
“Upper 3 Quartiles BW PCD4989g 20 mg/kg” (N=109): The remaining patients with BW available in this dose cohort.

Study PCD4989g subgroups by Cycle 1 observed Cₘₐₓ value
“PCD4989g 20 mg/kg >90%-ile Cₘₐₓ” (N=4): Patients in Study PCD4989g dosed with 20 mg/kg atezolizumab who had a Cₘₐₓ value in Cycle 1 that was above the 90^{th} percentile of the Cₘₐₓ predicted for 1680 mg atezolizumab IV.
“PCD4989g 20 mg/kg>90%-ile Cₘₐₓ” (N=134): Patients in Study PCD4989g dosed with 20 mg/kg atezolizumab who had a Cₘₐₓ value in Cycle 1 up to the 90^{th} percentile of the Cₘₐₓ predicted for 1680 mg atezolizumab IV.
“PCD4989g 20 mg/kg>mean Cₘₐₓ” (N=40): Patients in Study PCD4989g dosed with 20 mg/kg atezolizumab who had a Cₘₐₓ value in Cycle 1 that was above the mean value of the Cₘₐₓ predicted for 1680 mg atezolizumab IV.
“PCD4989g 20 mg/kg≤mean Cₘₐₓ” (N=98): Patients in Study PCD4989g dosed with 20 mg/kg atezolizumab who had a Cₘₐₓ value in Cycle 1 up to the mean value of the Cₘₐₓ predicted for 1680 mg atezolizumab IV.

Study PCD4989g 20 mg/kg subgroups as above, but using patients' Cycle 1 model-predicted Cₘₐₓ value instead of the observed Cₘₐₓ value

Study G028915 (OAK; N=609): Patients in Study G028915 who received atezolizumab doses of 1200 mg IV q3w.

Study G029294 (IMvigor211; N=459): Patients in Study G029294 who received atezolizumab doses of 1200 mg IV q3w.

### Safety Parameters

The AE terms for Study PCD4989g, IMvigor211, and OAK were coded to Preferred Terms using the Medical Dictionary for Regulatory Activities (MedDRA Version 20.1). AE severity was graded according to the National Cancer Institute Common Terminology Criteria for Adverse Events, Version 4.0 (NCI CTCAE v4.0) criteria.

For the purpose of this analysis, a set of comprehensive definitions using MedDRA-standardized SMQs, Sponsor-defined adverse event grouped terms (AEGTs), and High-Level Terms (HLTs) were used to identify AEs of special interest (AESIs) from the AE clinical database by medical concept. The medical concepts included atezolizumab-associated important identified risks and potential risks and class effects reported with other immune-checkpoint inhibitors.

Separate analyses were performed for AESIs that required the use of corticosteroid treatment. These AEs were identified using the following criteria:
AE term is in the grouping of AEs of special interest
Date of systemic corticosteroid initiation was on or up to 30 days after the AE onset date
Date of systemic corticosteroid initiation was before the AE resolution date

Corticosteroids were identified based on standard drug baskets. Systemic use was defined as any medication that did not have any of the following administration routes: auricular (otic), intravesical, intravitreal, nasal, ophthalmic, respiratory (inhalation), topical or vaginal.

In order to capture potential infusion-related reactions (IRRs), analyses were performed for AEs with onset during or within 24 hours of an atezolizumab infusion.

### Results

### Overview of Safety Profile

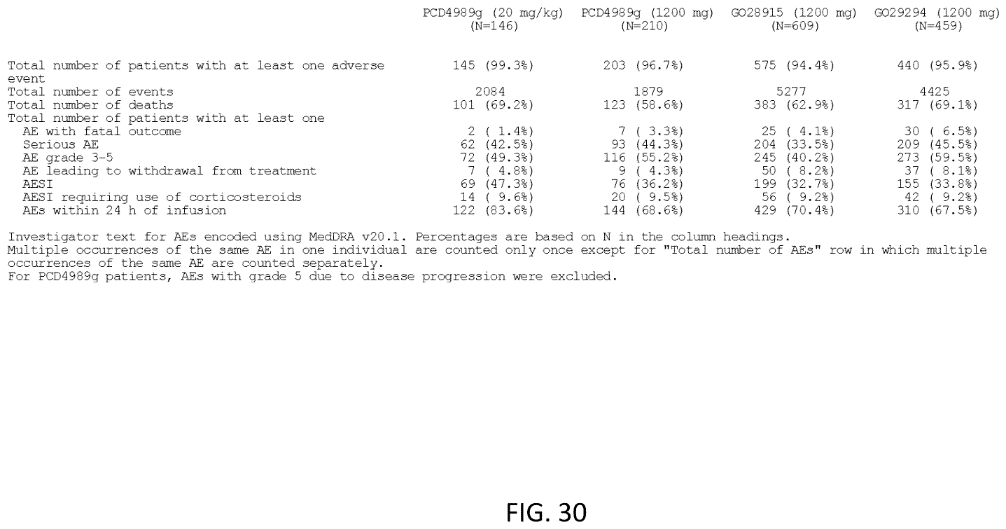

As shown in FIG. 30, the overall safety profile of atezolizumab given as a 20 mg/kg q3w dose was similar to that observed when given as a fixed 1200 mg q3w dose. Some differences were observed in the incidences across the treatment groups, with a higher incidence of AESIs and IRRs (AEs within 24 hours of infusion) in Study PCD4989g 20 mg/kg compared with the other treatment groups. For AESIs, immune-mediated rash as well as liver function test abnormalities was observed more frequently and for IRRs, the higher incidence in the 20 mg/kg treatment group was mainly accounted for by more events of arthralgia, rash, and chills.

### Common AEs

A similar proportion of patients experienced at least one AE of any grade for all treatment groups (99.3% PCD4989g 20 mg/kg vs. 96.7% PCD4989g 1200 mg vs. 94.4% OAK vs. 95.9% IMvigor211).

The most frequently observed AEs in the 20 mg/kg and 1200 mg treatment groups were similar. Those with a 10% difference in the 20 mg/kg cohort compared to any 1200 mg treatment group were generalized symptoms of dyspnea, nausea, and vomiting. Of these, the only event observed with a higher incidence in the 20 mg/kg cohort compared to all 1200 mg treatment groups was dyspnea (32.9% in PCD4989g (20 mg/kg, N=146); 18% is PCD4989g (1200 mg, N=210); 19.5% in OAK (1200 mg, N=609; 15.0% in IMvigor211 (1200 mg, N=459). These findings in individual AE incidences are considered secondary to underlying disease and unlikely due to potential exposure in the 20 mg/kg cohort.

### AEs by Intensity

A higher proportion of patients (59.5%) in IMvigor211 experienced at least one Grade 3 AE compared with the other treatment groups (49.3% PCD4989g 20 mg/kg vs. 55.2% PCD4989g 1200 mg vs. 40.2% OAK).

There was a 5% difference in incidence across treatment groups observed for anaemia (5.5% PCD4989g 20 mg/kg (N=146) vs. 5.7% PCD4989g 1200 mg (N=210) vs. 2.3% OAK 1200 mg (N=609) vs. 10.2% IMvigor211 1200 mg (N=459)) and urinary tract infection (0.7% PCD4989g 20 mg/kg (N=146) vs. 1.4% PCD4989g 1200 mg (N=210) vs. 0.2% OAK 1200 mg (N=609) vs. 5.7% IMvigor211 1200 mg (N=459)). Anaemia and urinary tract infection were reported at a higher frequency in IMvigor211, consistent with what is typically observed in a bladder cancer population.

### Serious AEs

Overall in all treatment groups, the proportion of patients who experienced at least one SAE was similar except for a lower incidence in OAK (42.5% PCD4989g 20 mg/kg vs. 44.3% PCD4989g 1200 mg vs. 33.5% OAK vs. 45.5% IMvigor211). Those with a 2% difference in the 20 mg/kg cohort compared to the 1200 mg treatment groups were PTs of dyspnoea, abdominal pain, pleural effusion and bone pain. Of these, the only event observed with a higher incidence in the 20 mg/kg cohort compared to any 1200 mg treatment group was dyspnea (6.2% PCD4989g 20 mg/kg (N=146); 3.8% PCD4989g 1200 mg (N=210); 2.1% OAK 1200 mg (N=609); 1.5% IMvigor211 1200 mg (N=459)). This finding in individual AE incidence is considered secondary to underlying disease and unlikely due to potential exposure in the 20 mg/kg cohort.

### AEs Leading to Withdrawal

The incidence of AEs leading to withdrawal in the 20 mg/kg treatment group was 4.8% compared with 4.3% for PCD4989g 1200 mg, 8.2% in OAK and 8.1% in IMvigor211.

There were 7 patients who discontinued atezolizumab in the 20 mg/kg cohort due to the following events: cardiac failure, death, asthenia, disease progression, bladder cancer, hypoxia and respiratory failure.

### AEs of Special Interest

Across all treatment groups, the proportion of patients with at least one AESI was higher in the 20 mg/kg treatment group (47.3%) compared with the 1200 mg treatment groups (36.2% PCD4989g 1200 mg vs. 32.7% OAK vs. 33.8% IMvigor211).

The most frequently reported events in all treatment groups were immune-mediated rash (17.1% PCD4989g 20 mg/kg vs. 6.7% PCD4989g 1200 mg vs. 9.7% OAK vs. 11.3% IMvigor211) and elevations in liver function tests (increased ALT [6.2% vs. 10.5% vs. 5.7% vs. 4.1%], and increased AST [6.2% vs. 11.4% vs. 6.2% vs. 4.4%]).

The higher incidence of AESIs in the 20 mg/kg treatment group was mainly accounted for by more events of immune-mediated rash, mostly Grade 1-2. The incidence and types of other AESIs were similar between the treatment groups.

The proportion of patients who received corticosteroids for an AESI was similar between all the treatment groups (9.6% PCD4989g 20 mg/kg vs. 9.5% PCD4989g 1200 mg vs. 9.2% OAK vs. 9.2% IMvigor211).

The most common (>2% of patients in any treatment group) AESIs requiring use of corticosteroids included pneumonitis (2.7% vs. 1.4% vs. 1.0% vs. 1.1%), increased ALT (0% vs. 2.9% vs. 1.0% vs. 0.4%), and increased AST (0% vs. 2.9% vs. 0.8% vs. 0.7%).

AEs Occurring within 24 Hours of Infusion

The proportion of patients who experienced at least one AE within 24 hours of infusion was higher in the 20 mg/kg treatment group (83.6%) compared with the 1200 mg treatment groups (68.6% PCD4989g 1200 mg vs. 70.4% OAK vs. 67.5% IMvigor211).

The higher incidence in the 20 mg/kg treatment group was mainly accounted for by more events of arthralgia (9.6% PCD4989g 20 mg/kg (N=146); 4.8% PCD4989g 1200 mg (N=210); 4.4% OAK 1200 mg (N=609); 3.3% IMvigor211 1200 mg (N=459)), rash (6.8% PCD4989g 20 mg/kg (N=146); 1.4%; 3.6% OAK 1200 mg (N=609); 2.6% IMvigor211 1200 mg (N=459)), and chills (5.5% PCD4989g 20 mg/kg (N=146); 1.0% PCD4989g 1200 mg (N=210); 1.6% OAK 1200 mg (N=609); 2.0% IMvigor211 1200 mg (N=459)). All events were reported as Grade 1-2. The incidence and types of other AEs occurring within 24 hours of infusion were generally similar between the treatment groups.

The higher incidence of AEs within 24 hours may be due to the data capture methodology: in Study PCD4989g, events associated with IRRs were captured as individual AEs and studies OAK and IMvigor211 captured the diagnosis of IRRs rather than individual AEs. In addition, the most common AEs reported within 24 hours of infusion were primarily generalized symptoms (e.g., deceased appetite, fatigue, asthenia) known to occur in this patient population. IRRs are a known risk for atezolizumab and other monoclonal antibodies. While arthralgia, rash, and chills may be a part of the cluster of symptoms typically associated with the development of an IRR, these generalized symptoms may also occur with concurrent illness or underlying disease. Additionally, these AEs were also reported outside of the 24-hour window of an infusion in all subgroups. Therefore, the development of IRRs is not considered to be associated with the 20 mg/kg treatment group.

### Example 8

Patient Subgroups in Study PCD4989g 20 mg/Kg by Cₘₐₓ During Cycle 1—Below or Above 90%-ile Value of Predicted Cₘₐₓ for 1680 mg Dose

The number of patients in the PCD4989g 20 mg/kg treatment group with an observed Cₘₐₓ value in Cycle 1 >90%-ile of the predicted Cₘₐₓ value for the 1680 mg dose was very small (n=4), hence no data interpretation or conclusions can be drawn from these analyses.

However, descriptive safety information for Grade >3 AEs for the four patients in the PCD4989g 20 mg/kg observed >90%-ile Cₘₐₓ subgroup are presented below:
Patient A died on Day 81 from malignant neoplasm progression, which was reported as a Grade 5 event. This patient also had a history of liver metastases and experienced a Grade 4 AE of blood bilirubin increased on Day 64 and Grade 3 AEs of ALT and AST increased, both on Day 70.
Patient B reported a Grade 3 AE of hypertension on Day 43, and a Grade 3 AE pathological fracture on Day 923.
Patient C reported Grade 3 AEs of increased international normalized ratio, fatigue, and dyspnoea on Days 44, 93, and 102, respectively.
Patient D died on Day 145 from malignant neoplasm progression, which was reported as a Grade 5 AE.

Overall, the results of PCD4989g 20 mg/kg Cycle 1 Cₘₐₓ subgroup analyses using observed Cₘₐₓ were very similar to those using the model-predicted Cₘₐₓ (Table 11).

TABLE 11
Overall Summary of Adverse Events in Patients Receiving Atezolizumab 20 mg/kg IV
q3w, Split by Observed or Modeled Cₘₐₓ during Cycle 1 (Below/Above 90%-ile Cₘₐₓ
predicted for 1680 mg Atezolizumab IV) (Atezolizumab-Treated Safety Evaluable Patients).

| | PCD4989g | | PCD4989g | | PCD4989g | | PCD4989g | |
|---|---|---|---|---|---|---|---|---|
| | (20 mg/kg) | | (20 mg/kg) | | (20 mg/kg) | | (20 mg /kg) | |
| | observed ≤90%- | | observed >90%- | | observed ≤90%- | | modeled >90%- | |
| | ile Cₘₐₓ (N = 134) | | ile Cₘₐₓ (N = 4) | | ile Cₘₐₓ (N = 142) | | ile Cₘₐₓ (N = 3) | |
| Total no. of | 133 | (99.3%) | 4 | (100.0%) | 141 | (99.3%) | 3 | (100.0%) |
| patients with at | | | | | | | | |
| least one AE | | | | | | | | |
| Total no. of | 90 | (67.2%) | 4 | (100.0%) | 98 | (69.0%) | 2 | (66.7%) |
| deaths | | | | | | | | |
| Total number | | | | | | | | |
| of patients with | | | | | | | | |
| at least one: | | | | | | | | |
| AE with fatal | 2 | (1.5%) | 0 | | 2 | (1.4%) | 0 | |
| outcome | | | | | | | | |
| Serious AE | 57 | (42.5%) | 1 | (25.0%) | 61 | (43.0%) | 0 | |
| AE Grade 3-5 | 63 | (47.0%) | 3 | (75.0%) | 71 | (50.0%) | 0 | |
| AEs leading to | 6 | (4.5%) | 0 | | 7 | (4.9%) | 0 | |
| withdrawal | | | | | | | | |
| from | | | | | | | | |
| treatment | | | | | | | | |
| AESI | 63 | (47.0%) | 2 | (50.0%) | 67 | (47.2%) | 2 | (66.7%) |
| AESI requiring | 12 | (9.0%) | 0 | | 14 | (9.9%) | 0 | |
| use of | | | | | | | | |
| corticosteroids | | | | | | | | |
| AEs within 24 | 113 | (84.3%) | 3 | (75.0%) | 121 | (85.2%) | 1 | (33.3%) |
| hours of | | | | | | | | |
| infusion | | | | | | | | |

### Example 9

Patient subgroups in study PCD4989g 20 mg/kg by Cₘₐₓ during Cycle 1—below or above mean predicted Cₘₐₓ for 1680 mg dose

In this Example, patient subgroups in study PCD4989g were analyzed for safety.

### Materials and Methods

AE frequencies were summarized for subgroups of patients: (1) from PCD4989g who received atezolizumab 20 mg/kg q3w based on Cₘₐₓ values in relation to predicted Cₘₐₓ for the 1680-mg q4w regimen and (2) from PCD4989g and OAK based on body weight quartiles (lowest quartile vs quartiles 2-4). In these analyses, whether or not AESIs required the use of corticosteroids was also specified.

### Results

Table 12 provides a safety summary for 20-mg/kg q3w atezolizumab-treated patients in PCD4989g, with observed Cₘₐₓ during cycle 1 relative to the mean predicted Cₘₐₓ of the 1680-mg q4w regimen. The overall safety profile was generally similar between the Study PCD4989g 20 mg/kg subgroup of patients with observed Cₘₐₓ during Cycle 1 mean and >mean predicted Cₘₐₓ value for the 1680 mg dose (Table 12). In general, AE frequencies were similar between these groups. Similar results were obtained in groups based on the PCD4989g patients' modeled Cₘₐₓ (i.e., individual predictions estimated by the popPK model) relative to the mean predicted Cₘₐₓ of the 1680-mg q4w regimen.

Overall, the results of PCD4989g 20 mg/kg observed Cₘₐₓ during Cycle 1 were similar to PCD4989g 20 mg/kg modeled Cₘₐₓ during Cycle 1.

TABLE 12
Overall Summary of Adverse Events in Patients Receiving Atezolizumab
20-mg/kg IV q3w (PCD4989g), Split by Observed or Modeled Cₘₐₓ
during Cycle 1 (Below/Above Mean Cₘₐₓ predicted for 1680-mg
Atezolizumab IV) (Atezolizumab-Treated Safety Evaluable Patients).

| | PCD4989g | PCD4989g | PCD4989g | PCD4989g |
|---|---|---|---|---|
| | (20 mg/kg) | (20 mg/kg) | (20 mg/kg) | (20 mg/kg) |
| | observed ≤ | observed > | modeled ≤ | modeled > |
| | mean Cₘₐₓ | mean Cₘₐₓ | mean Cₘₐₓ | mean Cₘₐₓ |
| | (N = 98) | (N = 40) | (N = 117) | (N = 28) |
| Total no. of | 97 (99.0%) | 40 (100.0%) | 116 (99.1%) | 28 (100.0%) |
| patients with | | | | |
| at least one | | | | |
| AE | | | | |
| Total no. of | 70 (71.4%) | 24 (60.0%) | 81 (69.2%) | 19 (67.9%) |
| deaths | | | | |
| Total number | | | | |
| of patients | | | | |
| with at least | | | | |
| one: | | | | |
| AE with fatal | 2 (2.0%) | 0 | 2 (1.7%) | 0 |
| outcome | | | | |
| Serious AE | 43 (43.9%) | 15 (37.5%) | 49 (41.9%) | 12 (42.9%) |
| AE Grade 3-5 | 52 (53.1%) | 14 (35.0%) | 61 (52.1%) | 10 (35.7%) |
| AEs leading | 5 (5.1%) | 1 (2.5%) | 7 (6.0%) | 0 |
| to withdrawal | | | | |
| from | | | | |
| treatment | | | | |
| AESI | 47 (48.0%) | 18 (45.0%) | 54 (46.2%) | 15 (53.6%) |
| AESI | 8 (8.2%) | 4 (10.0%) | 12 (10.3%) | 2 (7.1%) |
| requiring | | | | |
| use of corti- | | | | |
| costeroids | | | | |
| AEs within | 78 (79.6%) | 38 (95.0%) | 96 (82.1%) | 26 (92.9%) |
| 24 hours of | | | | |
| infusion | | | | |

Atezolizumab-treated safety-evaluable patients were included.
AE = adverse event, AESI = adverse event of special interest, Cₘₐₓ = maximum serum atezolizumab concentration, q3w = every 3 weeks, q4w = every 4 weeks.

A similar proportion of patients experienced at least one AL of any grade for both treatment subgroups (99.0% for observed≤mean Cₘₐₓ vs. 100.0% for observed>mean Cₘₐₓ). AEs of any grade with a difference of ≥10% incidence were decreased appetite (more common in the >mean Cₘₐₓ subgroup) and anaemia (more common in the ≤mean Cₘₐₓ subgroup).

A higher proportion of patients (53.1%) in the observed ≤mean Cₘₐₓ subgroup experienced at least one Grade 3 AE compared with the observed >mean Cₘₐₓ subgroup (35.0%).

The most common (>5% of patients in either treatment group) Grade ≥3 AEs reported by PTs were dyspnoea, anaemia, and fatigue (Table 13). There were no Grade ≥3 AEs which occurred at a higher (5%) incidence in the >mean Cₘₐₓ subgroup; events which occurred more commonly in the mean Cₘₐₓ subgroup than the observed >mean Cₘₐₓ subgroup were dyspnoea and anaemia.

TABLE 13
Grade ≥ 3 AEs Reported in >5% of Patients in Any Subgroup
(Atezolizumab-Treated Safety Evaluable Patients)

| | PCD4989g (20 mg/kg) | PCD4989g (20 mg/kg) |
|---|---|---|
| MedDRA | observed ≤ mean | observed > mean |
| Preferred Term | Cₘₐₓ(N = 98) | Cₘₐₓ (N = 40) |
| Dyspnoea | 10 (10.2%) | 1 (2.5%) |
| Anaemia | 8 (8.2%) | 0 |
| Fatigue | 5 (5.1%) | 1 (2.5%) |

Analysis of Serious Adverse Events in Patients with Cycle 1 Cₘₐₓ Below or Above Mean Value of Predicted Cₘₐₓ for 1680 mg Dose

A similar proportion of patients experienced at least one SAE for both treatment subgroups (43.9% observed mean Cₘₐₓ vs. 37.5% observed >mean Cₘₐₓ). Dyspnoea occurred more commonly in the mean Cₘₐₓ subgroup than the observed >mean Cₘₐₓ subgroup (Table 14).

TABLE 14
Serious Adverse Events Reported in ≥5% of Patients in Any
Subgroup (Atezolizumab-Treated Safety Evaluable Patients).

| MedDRA | PCD4989g (20 mg/kg) | PCD4989g (20 mg/kg) |
|---|---|---|
| Preferred | observed ≤ mean | observed > mean |
| Term | Cₘₐₓ (N = 98) | Cₘₐₓ (N = 40) |
| Dyspnoea | 8 (8.2%) | 0 |
| Bone Pain | 1 (1.0%) | 2 (5.0%) |
| Pyrexia | 1 (1.0%) | 2 (5.0%) |

Analysis of Adverse Events that LED to Withdrawal in Patients with Cycle 1 Cₘₐₓ Below or Above Mean Value of Predicted Cₘₐₓ for 1680 mg Dose

Overall, few patients discontinued atezolizumab due to AEs (5.1% observed mean Cₘₐₓ vs. 2.5% observed >mean Cₘₐₓ). The events leading to withdrawal were reported in single patients. The five patients in mean Cₘₐₓ discontinued due to cardiac failure, asthenia, death, disease progression, hypoxia and respiratory failure. One patient in the >mean Cₘₐₓ discontinued due to disease progression.

Analysis of Adverse Events of Special Interest in Patients with Cycle 1 Cₘₐₓ Below or Above Mean Value of Predicted Cₘₐₓ for 1680 mg Dose

Overall, a similar proportion of patients in both subgroups experienced at least one AESI (48.0% observed≤mean Cₘₐₓ vs. 45.0% observed>mean Cₘₐₓ). Immune-mediated rash (19.4% vs. 12.5%) and abnormalities in liver function tests (increased ALT 7.1% vs 5.0%; increased AST 6.1% vs 7.5%) were the most frequently reported AESIs in both subgroups.

Overall, a similar proportion of patients in both subgroups received corticosteroids for AESIs (8.2% observed≤mean Cₘₐₓ vs. 10.0% observed>mean Cₘax). The AESIs requiring use of corticosteroids reported most commonly were pneumonitis (2 patients in each subgroup) and rash (2 patients vs. 0 patients).

Analysis of adverse events occurring within 24 hours of infusion in patients with cycle 1 Cₘₐₓ below or above mean value of predicted Cₘₐₓ for 1680 mg dose

A higher proportion of patients (95.0%) in the observed >mean Cₘₐₓ subgroup experienced an AE within 24 hours of infusion compared with the observed mean Cₘₐₓsubgroup (79.6%).

Events which occurred more frequently (>5%) in the observed >mean Cₘₐₓ subgroup were nausea, asthenia, and diarrhea (Table 15).

TABLE 15
Common Adverse Events Occurring Within 24 Hours of
Infusion Reported in >10% of Patients in Any Subgroup
(Atezolizumab-Treated Safety Evaluable Patients).

| MedDRA | PCD4989g (20 mg/kg) | PCD4989g (20 mg/kg) |
|---|---|---|
| Preferred | observed ≤ mean | observed > mean |
| Term | Cₘₐₓ (N = 98) | Cₘₐₓ (N = 40) |
| Fatigue | 12 (12.2%) | 7 (7.5%) |
| Constipation | 9 (9.2%) | 5 (12.5%) |
| Nausea | 8 (8.2%) | 6 (15.0%) |
| Asthenia | 6 (6.1%) | 5 (12.5%) |
| Diarrhoea | 4 (4.1%) | 5 (12.5%) |

### Safety by Dose Group

Observed safety data were evaluated by exposure subgroups.

Table 16 provides a summary for PCD4989g by atezolizumab exposure by dose group. In a dose range from 10 mg/kg q3w to 20 mg/kg q3w and 1200 mg q3w, the median treatment duration ranged from 2.07 to 9.48 months, and the median number of doses ranged from 4 to 14.5.

TABLE 16
Atezolizumab exposure by dose group: atezolizumab-treated
patients from PCD4989g.

| | 10 mg/kg | 15 mg/kg | 20 mg/kg | 1200 mg |
|---|---|---|---|---|
| | q3w IV | q3w IV | q3w IV | q3w IV |
| | (n = 36) | (n = 236) | (n = 146) | (n = 228) |
| Treatment | | | | |
| duration | | | | |
| n | 36 | 236 | 146 | 228 |
| Mean (SD) | 15.38 (18.17) | 10.44 (15.93) | 8.55 (11.98) | 4.43 (7.18) |
| Median | 9.48 | 3.42 | 4.62 | 2.07 |
| (Min-Max) | (0.0-67.0) | (0.0-64.7) | (0.0-69.1) | (0.0-40.7) |
| Number | | | | |
| of doses | | | | |
| n | 36 | 236 | 146 | 228 |
| Mean (SD) | 16.5 (15.3) | 14.0 (19.3) | 11.5 (13.5) | 7.1 (10.1) |
| Median | 14.5 (1-61) | 6 (1-79) | 7 (1-96) | 4 (1-60) |
| (Min-Max) | | | | |

Duration indicated in number of months, SD = standard deviation, q3w = every 3 weeks

Table 17 provides a safety summary for PCD4989g patients by dose group. The overall safety profile was consistent among 15 mg/kg q3w, 20 mg/kg q3w, and 1200 mg q3w groups. Patients in the 10 mg/kg q3w dose group demonstrated increased frequency of serious adverse events (AEs) and treatment-related AEs relative to the other dose groups. This may be due to the longer safety follow-up and the lower number of patients in this dose group relative to the other dose groups.

TABLE 17
AE summary by dose group: atezolizumab-treated
patients from PCD4989g.

| | 10 mg/kg | 15 mg/kg | 20 mg/kg | 1200 mg |
|---|---|---|---|---|
| Patients with ≥ 1 | q3w IV | q3w IV | q3w IV | q3w IV |
| indicated AE, n (%) | (n = 36) | (n = 236) | (n = 146) | (n = 228) |
| Any AE¹ | 35 (97.2) | 232 (98.3) | 145 (99.3) | 225 (98.7) |
| AE with fatal outcome | 1 (2.8) | 3 (1.3) | 2 (1.4) | 7 (3.1) |
| Serious AE | 20 (55.6) | 115 (48.7) | 65 (44.5) | 103 (45.2) |
| Serious AE leading to | 2 (5.6) | 9 (3.8) | 4 (2.7) | 8 (3.5) |
| treatment withdrawal | | | | |
| Serious AE leading to | 7 (19.4) | 41 (17.4) | 22 (15.1) | 41 (18.0) |
| dose interruption | | | | |
| AE leading to | 2 (5.6) | 16 (6.8) | 33 (22.6) | 69 (30.3) |
| withdmwal from | | | | |
| treatment | | | | |
| AE leading to dose | 13 (36.1) | 66 (28.0) | 33 (22.6) | 69 (30.3) |
| interruption | | | | |
| Related AE | 31 (86.1) | 174 (73.7) | 110 (75.3) | 141 (61.8) |
| Related AE leading to | 1 (2.8) | 11 (4.7) | 3 (2.1) | 5 (2.2) |
| treatment withdrawal | | | | |
| Related AE leading to | 4 (11.1) | 27 (11.4) | 17 (11.6) | 25 (11.0) |
| dose interruption | | | | |

¹Per PCD4989g protocol, all adverse events were collected after treatment initiation until 90 days following the last administration of study treatment or until study discontinuation/termination or until initiation of subsequent anti-cancer therapy, whichever occurred first. Patients were contact at 60 and 90 days after the last dose of study treatment to determine if any new adverse events had occurred. After this period, investigators reported only serious adverse events that were felt to be related to prior study treatment.
AE = adverse event, q3w = every 3 weeks.

### Safety by Body Weight

Observed safety data were evaluated by exposure and body weight subgroups.

Table 18 provides a safety summary for PCD4989g and OAK patients by body weight. Median body weight in the 20-mg/kg treatment group in PCD4989g was 78.2 kg (Q1-Q3, 63.7-93.0 kg), and the overall safety profile was generally similar between patients in the lowest (n 37) and upper 3 (n 109) body weight quartiles. A higher incidence of grade 3 to 5 AEs (48.7% vs 37.3%) in the lowest body weight quartile subgroup was observed, which was due to grade 3 As (38.8% vs 27.8%). Evaluation of grade 3 ALs did not identify any individual AE preferred term with a ≥2% difference between subgroups. Serious ALs with a 5% difference between subgroups included fatigue and asthenia (both common to malignancy) as well as pneumonia and cardiac tamponade (known complications of thoracic cancers), with all such events occurring infrequently. In the lowest body weight subgroup, only asthenia and respiratory complications led to study treatment withdrawal; no action with respect to study treatment was taken for the other events. To assess the impact of body weight in a larger cohort of patients, AL data from OAK (1200-mg q3w dosing) were also analyzed. Median body weight was 71.0 kg (Q1-Q3, 59.5-82.2 kg). No differences between the lowest (n 152) and upper 3 (n 442) body weight quartiles were observed.

TABLE 18
AE summary by body weight: atezolizumab-treated patients from
PCD4989g and OAK.

| | Patients from indicated study, dosing | | | |
|---|---|---|---|---|
| | subgroup and body weight quartile(s) | | | |
| | PCD4989g | PCD4989g | OAK | OAK |
| Patients with ≥1 | (20 mg/kg), | (20 mg/kg), | (1200 mg), | (1200 mg), |
| indicated AE, | lowest | upper 3 | lowest | upper 3 |
| n (%) | (n = 37) | (n = 109) | (n = 152) | (n = 442) |
| Any AE | 37 (100.0) | 108 (99.1) | 142 (93.4) | 418 (94.6) |
| Total deaths | 24 (64.9) | 77 (70.6) | 98 (64.5) | 277 (62.7) |
| AE with fatal | 1 (2.7) | 1 (0.9) | 5 (3.3) | 20 (4.5) |
| outcome | | | | |
| Serious AE | 17 (45.9) | 45 (41.3) | 51 (33.6) | 151 (34.2) |
| Grade 3-5 AE | 21 (56.8) | 51 (46.8) | 74 (48.7) | 165 (37.3) |
| AE leading to | 3 (8.1) | 4 (3.7) | 16 (10.5) | 32 (7.2) |
| treatment | | | | |
| withdrawal | | | | |
| AESI | 15 (40.5) | 54 (49.5) | 45 (29.6) | 150 (33.9) |
| AESI requiring | 3 (8.1) | 11 (10.1) | 11 (7.2) | 44 (10.0) |
| corticosteroids | | | | |
| AE within 24 | 32 (86.5) | 90 (82.6) | 99 (65.1) | 321 (72.6) |
| hours | | | | |
| of infusion | | | | |

Atezolizumab-treated safety-evaluable patients were included.
AE = adverse event, AESI = adverse event of special interest.

### Example 10

### Analysis of Immunogenicity

The immunogenicity of atezolizumab was evaluated in Studies PCD4989g, JO28944, IMvigor210, IMvigor211, BIRCH, POPLAR, FIR, and OAK.

Analysis of the post-baseline treatment-emergent ADA incidence for 20 mg/kg q3w in Study PCD4989g vs. 1200 mg q3w in OAK vs. 1200 mg q3w in IMvigor 211 revealed no apparent increase in treatment-emergent ADA incidence with a 20 mg/kg dose (Table 19).

TABLE 19
Post-Baseline Treatment-Emergent ADA Incidence for q3w Dosing:
20 mg/kg in PCD4989g, 1200 mg in OAK and IMvigor 211.

| | PCD4989g | OAK | IMvigor211 |
|---|---|---|---|
| | 20 mg/kg | 1200 mg | 1200 mg |
| | dose | dose | dose |
| Post-baseline evaluable | 137 | 565 | 427 |
| patients | | | |
| No. of patients positive | 27 (19.7%) | 172 (30.4%) | 142 (33.3%) |
| for ADA | | | |
| Treatment-induced^{a} ADA | 27 | 171 | 139 |
| Treatment-enhanced^{b} ADA | 0 | 1 | 3 |
| No. of patients negative | 110 (80.3%) | 393 (69.6%) | 285 (66.7%) |
| for ADA | | | |
| Treatment-unaffected^{c} | | | |
| ADA | 5 | 19 | 6 |

ADA = anti-drug antibody.
^{a}Treatment-induced ADAs: Patients who had a baseline-negative or missing baseline ADA result and developed anti-atezolizumab antibodies at any time after initial drug administration.
bTreatment-enhanced ADAs: Patients who had a baseline-positive ADA result in whom the assay signal was enhanced (greater than baseline titer by ≥0.60 titer units) at any time after initial drug administration.
^{c}Treatment-unaffected ADAs: Patients who had a baseline-positive ADA result in whom the assay signal was not enhanced (not greater than baseline titer by ≥0.60 titer units) at any time after initial drug administration. These patients are considered postbaseline negative for ADAs.

The presence of atezolizumab in ADA serum samples can interfere with ADA detection. In validation experiments, the ADA assay was able to detect 500 ng/mL of surrogate positive control anti-atezolizumab antibodies in the presence of 200 μg/mL atezolizumab. The following percentage of post-baseline ADA samples had atezolizumab concentrations that were below 200 μg/mL, which is the drug tolerance level of the ADA assay based on the surrogate positive control: Study PCD4989g 80.2%, IMvigor210 86.0%, IMvigor211 88.2%, BIRCH 82.8%, POPLAR 89.6%, FIR 86.9%, and OAK 81.9%.

Immunogenicity data are highly dependent on the sensitivity and specificity of the test methods used. Additionally, the observed incidence of a positive result in a test method may be influenced by several factors, including timing of sample collection, drug interference, concomitant medication and the underlying disease. Therefore, comparison of the incidence of antibodies to atezolizumab with the incidence of antibodies to other products may be misleading.

### Impact of Treatment-Emergent ADA Presence on Atezolizumab Pharmacokinetics in UC Patients

Despite the incidence of treatment-emergent ADA positivity (ranging from 16.7% to 41.9% in Study PCD4989g, JO28944, IMvigor210, and IMvigor211), the NCA analysis indicated that ADA positivity had a minor impact on atezolizumab exposure at doses from 10 to 20 mg/kg including the fixed dose of 1200 mg q3w. The popPK analysis also indicates that the presence of treatment-emergent ADA has a minor impact on atezolizumab exposure. Patients who were ADA-positive had a relatively small increase in atezolizumab clearance of 16% compared to ADA-negative patients (e.g., see Example 1). In all studies, for patients receiving atezolizumab doses ≥10 mg/kg, Cₘᵢₙ was maintained in excess of the target serum concentration of 6 μg/mL in the ADA-positive patients.

### Impact of Treatment-Emergent ADA Presence on Atezolizumab Pharmacokinetics in NSCLC Patients

Across the different clinical studies, treatment-emergent ADA positivity did not appear to have a major effect on atezolizumab concentrations and pharmacokinetics although there was a trend for lower Cₘᵢₙ values in the ADA-positive subgroup. The popPK model determined that the ADA-positive subgroup had a drug clearance 16% higher than ADA-negative patients, which accounts for the trend to lower exposure in ADA-positive patients (e.g., see Example 1). In all studies, for doses 10 mg/kg, Cₘᵢₙ remained well in excess of the target serum concentration of 6 μg/mL in the ADA-positive patients.

### Impact of Treatment-Emergent ADA Presence on Atezolizumab Efficacy in UC Patients

A review of ORRs across Study PCD4989g, IMvigor210, and IMvigor211 for UC did not demonstrate that treatment-emergent ADA positivity is consistently associated with a lower ORR. Analysis of IMvigor211 revealed no clinically relevant differences between ADA-positive and ADA-negative patients in all patients or in IC1/2/3 or IC2/3 groups, with overlapping 95% CIs for the outcome measures (OS, PFS, ORR, and DOR).

### Impact of Treatment-Emergent ADA Presence on Atezolizumab Efficacy in NSCLC Patients

ORRs were generally comparable between ADA-positive and ADA-negative patients and where there were numerical differences, the 95% CI were overlapping with no consistent increase or decrease in ORRs across studies. Overall, there was no apparent impact of treatment-emergent ADA on efficacy based on ORR, with overlapping confidence intervals for ADA-negative and ADA-positive patients.

Overall no clinical relevant differences were observed between ADA-positive patients and ADA-negative patients. OS was not mature for POPLAR; POPLAR median PFS was numerically higher in ADA-positive patients compared with ADA-negative patients, but the 95% CIs for PFS overlapped. For the OAK study, although the median OS, landmark OS rates, and median PFS were numerically higher in ADA-negative patients compared with ADA-positive patients, the 95% CIs of these outcome measures overlapped.

### Impact of Treatment-Emergent ADA Presence on Atezolizumab Safety

The post-baseline incidence of treatment-emergent ADA (treatment induced and enhanced) was 42.5% (540/1272) in the All Patients population, which is consistent with observations in the All UC population (41.9% [161/384]) and the All NSCLC population (42.7% [379/888]).

The incidence of all grade AEs, Grade 5 AEs, AEs leading to treatment withdrawal, AEs leading to dose interruption, and AESIs was similar irrespective of post-baseline ADAs status (negative or positive). Some numerical differences were observed in Grade 3-4 AEs (38.4% in ADA-negative vs. 44.3% in ADA-positive patients), which was mainly driven by AEs reported in the Gastrointestinal disorders SOC in the ADA-positive patients (5.7% vs. 8.5%), but no individual PTs could be identified to explain this difference. The incidence of SAEs was higher in ADA-positive patients (40.2%) compared with ADA-negative patients (33.5%), but this difference was not driven by any specific SOC or individual AE preferred term.

In the All Patients population, the incidence of hypersensitivity and IRRs (MedDRA AE PTs) was low and consistent between ADA-positive and ADA-negative patients. Hypersensitivity events were reported in 18 patients (1.4%): 8 ADA-negative (1.1%) and 10 ADA-positive (1.9%) patients. Infusion-related reactions occurred in 20 patients (1.6%): 11 ADA-negative (1.5%) and 9 ADA-positive (1.7%) patients.

### Example 11

Assessment of Toxicological Safety Margin with Predicted Atezolizumab 1680 mg q4w Fixed Dose

The 1680-mg q4w dosing regimen represents a 1-mg/kg, or 5%, higher dose on a mg/kg basis than the previous highest dose administered to patients. As noted in the previous Examples, predicted Cₘᵢₙ at Cycle 1 and steady-state for 1680 mg q4w is lower than that predicted for 20 mg/kg q3w. The predicted Cₘₐₓ at Cycle 1 and at steady-state is 12% and 0.8% higher than it is for the 20-mg/kg q3w dosing regimen, respectively. In light of the higher predicted Cₘₐₓ for the 1680 mg q4w, a reassessment of the atezolizumab toxicology margins was carried out.

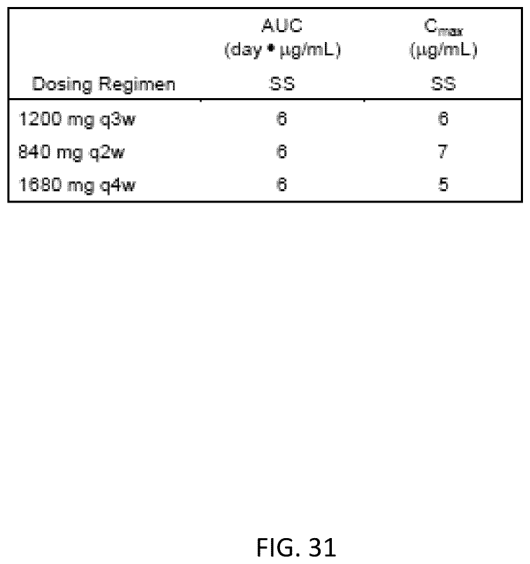

The toxicological safety margins of the 840-mg q2w and 1680-mg q4w regimens were assessed using the highest tolerated dose of 50 mg/kg in a repeat-dose toxicity study in cynomolgus monkey and the human PK parameters at the current 1200-mg q3w dose level (FIG. 31). Safety factors for atezolizumab were calculated using the following methods:
Exposure AUC-based: Comparison of predicted AUC at the proposed clinical dose to the AUC calculated at the highest tolerated 50-mg/kg dose level in the repeat-dose cynomolgus monkey toxicology study, respectively (AUCAnimal/AUCHuman). In the 26-week repeat dose toxicity study in cynomolgus monkeys (Study 13-3278), the animals were dosed weekly at the highest tolerated dose of 50 mg/kg (i.e., more frequently compared to the q3w regimen in patients). Hence, over a 3-week period (to match q3w dosing regimen in patients), the monkeys received a total dose of 150 mg/kg (i.e., 50 mg/kg once weekly×3 weeks). Using this total dose of 150 mg/kg and a monkey CL value of 3.7 mL/day/kg, the AUC in monkeys was calculated to be 40,500 day·μg/mL (i.e., 150 mg/kg divided by 3.7 mL/day/kg). Comparing this calculated monkey exposure of 40,500 day·μg/mL to human steady state exposure of 6,409 day·μg/mL (from 1200 mg given q3w, Study PCD4989g), gives a safety margin of 6× (i.e., 40,500 divided by 6,409). Similar calculations were performed for the 840-mg q2w and 1680-mg q4w regimens using simulated clinical AUC (FIG. 31).
Concentration Cₘₐₓ-based: Comparison of the Cₘₐₓ reported in Study PCD4989g for the 1200-mg q3w regimen or simulated clinical Cₘₐₓ for the proposed 840-mg q2w and 1680-mg q4w regimens, to that observed at the highest tolerated dose of 50 mg/kg in the repeat-dose cynomolgus monkey study, respectively (Cₘₐₓ Animal/Cₘₐₓ Human) (FIG. 31). Cₘₐₓ following 27 IV doses of atezolizumab at 50 mg/kg to cynomolgus monkey was 3,680 μg/mL.

As shown above and based on exposure and concentration analyses, the pharmacokinetics and toxicokinetics of atezolizumab in the cynomolgus monkey provide adequate safety margins to support the 840-mg q2w and 1680-mg q4w clinical dosing regimens.

### Example 12

Interchangeability of the 1200 mg q3w, 840-Mg q2w, and 1680 mg q4w Dosing Regimens

The efficacy and safety profile of the approved atezolizumab 1200-mg q3w dosing regimen has been established, e.g., in patients with 2L NSCLC, 2L mUC, and/or in 1L cisplatin-ineligible mUC patients. To offer greater convenience and flexibility in patient care, dosing regimens of 840-mg q2w and 1680-mg q4w as IV infusions are provided herein. These new dosing regimens are intended to be interchangeable with the atezolizumab 1200-mg q3w dosing regimen.

An assessment of available atezolizumab monotherapy PK and ER data for UC and NSCLC has been conducted based on eight clinical studies as has been described in the preceding Examples. Key findings included:
No clinically meaningful exposure-efficacy or exposure-safety relationships were identified when atezolizumab was administered as monotherapy to patients with mUC or NSCLC.
Based on model-based simulations of 840-mg q2w and 1680-mg q4w dosing regimens, the predicted exposures are within range of the observed exposure with 1200 mg q3w atezolizumab. The predicted Cₘᵢₙ concentration of the 840-mg q2w and the 1680-mg q4w dosing regimens at Cycle 1 and at steady-state are above the target Cₘᵢₙ concentration of 6 g/mL.
The overall treatment-emergent incidence of ADA to atezolizumab did not have clinically meaningful impact on PK, efficacy, or safety. There was no apparent increase in the incidence of treatment-emergent ADA with a 20 mg/kg dose.

Based on safety data from Studies PCD4989g, OAK, and IMvigor211:
Patients with an observed Cₘₐₓ>759 μg/mL, which is the expected Cₘₐₓ for atezolizumab 1680 mg q4w, tolerated the dosing regimen well and no differences in the safety profile were noted when compared to patients with Cₘₐₓ≤759 μg/mL.
The overall safety profile was similar between patients who received a 20-mg/kg q3w dosing regimen and 1200-mg q3w dosing regimen.
No meaningful differences were observed in the safety profiles of patients with lower or higher BW.

A new atezolizumab 840-mg presentation has been developed to support the atezolizumab 840-mg q2w and 1680-mg q4w dosing schedules. These additional dosing schedules utilize the new 840-mg presentation (one vial of 840 mg atezolizumab for the 840-mg q2w schedule; two vials of 840 mg atezolizumab for the 1680-mg q4w schedule). There are no changes to either the atezolizumab formulation (i.e., identical strength with a concentration of 60 mg/mL active substance in both the 1200-mg and the 840-mg presentation) nor excipients and composition of the primary packaging material with the new presentation.

Based on the results from PK modeling and simulation, ER assessments, safety analyses, and immunogenicity data, it is not anticipated that there will be clinically meaningful differences in exposure, efficacy, and safety between the proposed atezolizumab doses of 840-mg q2w and 1680 mg q4w and the currently approved dose of 1200 mg q3w in NSCLC and UC.

Based on available evidence, it is reasonable to conclude that the 1200-mg q3w, 840-mg q2w, and 1680-mg q4w dosing regimens can be considered interchangeable. The use of “interchangeable” here is meant to indicate that any atezolizumab dosing regimen can be substituted for another, and the selection of specific dosing regimens can be based on patient-specific factors such as the coordination of atezolizumab dosing with other aspects of patient care.

### CONCLUSION

Results from this study support the interchangeable use of 840-mg q2w, 1200-mg q3w, and 1680-mg q4w dosing regimens for atezolizumab, as they are anticipated to demonstrate comparable efficacy and safety profiles while offering patients greater flexibility and convenience in their treatment.

The overall benefit/risk profile of the proposed 840-mg q2w and 1680-mg q4w dosing regimens are comparable to that of the currently approved 1200-mg q3w dosing regimen, which has been deemed positive in patients with NSCLC and UC. The new 840-mg q2w and 1680-mg q4w dosing regimens, in addition to the 1200-mg q3w dosing regimen, offer greater flexibility and convenience in patient care, for example, by reducing treatment burden and improving quality of life, as well as improving resource utilization at treatment facilities.

The results provided above show that no significant ER relationships were observed for safety or efficacy. Predicted exposures for 840 mg q2w and 1680 mg q4w were comparable to 1200 mg q3w and the MAD and consistent with observed PK data from IMpassion130. Observed safety was similar between patients with a Cₘₐₓ above and below the predicted Cₘₐₓ for 1680 mg q4w and between patients in the lowest and upper 3 body weight quartiles.

Briefly, data from all evaluated dose levels using a q3w dosing frequency, including 1200 mg q3w and 20 mg/kg q3w (the MAD in the phase 1 study PCD4989g), demonstrated that there was not a clinically meaningful exposure-efficacy or exposure-safety relationship. These data suggested that if a new dosing regimen achieves an exposure within the observed exposure range for 1200 mg q3w or 20 mg/kg q3w, it is not likely to impact efficacy or safety. PK simulations suggested that the new dosing regimens, 840 mg q2w and 1680 mg q4w, are predicted to achieve generally comparable exposure to that of the currently approved regimen of 1200 mg q3w and are within range of observed exposures from the 1200-mg q3w and 20-mg/kg dose levels. Further characterization of the observed safety profile of patients with a Cₘₐₓ above and below the predicted Cₘₐₓ of the 1680-mg q4w regimen also support that the safety profile of 1680 mg q4w is anticipated to be similar to the clinical experience with the q3w regimen.

The PK simulations of a 1680-mg q4w dosing regimen also indicated comparable overall exposure to the currently approved regimen of 1200 mg q3w, while the predicted steady-state Cₘᵢₙ was 6% lower than that for the currently approved regimen; this concentration also exceeded the target concentration. A small increase in cycle 1 and steady-state geometric mean Cₘₐₓ (12% and 0.8%, respectively) was anticipated when compared with the 20-mg/kg dose; however, the predicted Cₘₐₓ for the 1680-mg q4w regimen was within the range observed in the phase 1 study PCD4989g. Further, patients from PCD4989g treated at 20 mg/kg q3w had comparable safety regardless of whether their Cₘₐₓ was above or below the predicted cycle 1 values for the 1680-mg q4w regimen.

Similar to observations with the 1200-mg q3w regimen (Stroh et al., (2017) Clin Pharmacol Ther doi: 10.1002/cpt.587), the impact of body weight on exposure is not anticipated to be clinically meaningful for the 840-mg q2w or 1680-mg q4w regimens, as the predicted exposures for patients with low and high body weight are within range of observed exposures from the 1200-mg q3w and 20-mg/kg dose levels. These results are also further supported by a safety analysis from studies PCD4989 and OAK by body weight, which demonstrated that the overall observed safety profile was generally similar between patients in the lowest and upper 3 body weight quartiles.

The maintenance of Cₘᵢₙ levels of a protein therapeutic is considered to not only provide the most consistent disease control but also to minimize the likelihood of development of ADAs. Clinical data from TNF inhibitor studies show that episodic exposure to a protein therapeutic (i.e., exposure followed by complete washout, followed by re-exposure) is more likely to induce an immune response than the consistent presence of the same protein at the same level. The predicted Cₘᵢₙ levels of the 840 mg q2w and 1680 q4w regimens are well in excess of the target concentration (6 μg/mL) and are within range of Cₘᵢₙ values of the approved 1200 mg q3w regimen. Therefore, it is not anticipated that the 840 mg q2w or 1680 mg q4w regimens would result in a complete washout and re-exposure cycle that would lead to a higher immunogenicity rate than the approved 1200 mg q3w regimen.

The ability to administer atezolizumab at a less frequent dosing regimen (i.e., 1680-mg q4w) provides patients, caregivers, and healthcare providers greater flexibility and convenience. As atezolizumab is administered intravenously, the 1680-mg q4w dosing regimen is likely to reduce the time needed to receive treatment (e.g., number of visits to treatment centers) relative to a regimen dosed more frequently. In addition, the ability to switch regimens throughout treatment will also allow for greater flexibility as the dosing schedule can be matched to meet the evolving needs of each individual patient.

Atezolizumab regimens of 840 mg q2w and 1680 mg q4w are expected to have comparable efficacy and safety as the approved regimen of 1200 mg q3w, given that the predicted exposures are within the range of observed exposures and there is no clinically meaningful ER relationship. Further, as atezolizumab PK are consistent between indications and in combination with various agents evaluated (including, but not limited to, chemotherapy, antineoplastic drugs, and tyrosine kinase inhibitors), these results are applicable across indications where atezolizumab is administered either as monotherapy or in combination.

In summary, atezolizumab regimens of 840 mg q2w and 1680 mg q4w are expected to have comparable efficacy and safety as the approved regimen of 1200 mg q3w, supporting their interchangeable use and offering patients greater flexibility.

Thus, the analyses provided herein support the interchangeable use of atezolizumab dosing regimens of 840 mg q2w, 1200 mg q3w, and 1680 mg q4w, offering patients greater flexibility and convenience during their atezolizumab treatment. These data contributed to the expansion of atezolizumab dosing regimens for certain types of cancers by the FDA (Tecentriq (atezolizumab) [package insert]. South San Francisco, CA: Genentech, Inc.; 2019. South San Francisco, CA, USA: Genentech, Inc).

1-47. (canceled)

48. A method for treating a human patient having a cancer, wherein the method comprises administering to the patient atezolizumab at a dose of 1680 mg every 4 weeks.

49. The method of claim 48, wherein the method comprises administering the atezolizumab by intravenous infusion.

50. The method of claim 48, wherein the cancer is a locally advanced or a metastatic cancer.

51. The method of claim 48, wherein the atezolizumab is administered as a first-line treatment.

52. The method of claim 48, wherein the cancer is a lung cancer, a liver cancer, a melanoma, or a sarcoma.

53. The method of claim 52, wherein the cancer is a lung cancer.

54. The method of claim 53, wherein the lung cancer is a non-small cell lung cancer (NSCLC) or a small cell lung cancer (SCLC).

55. The method of claim 53, wherein the lung cancer is a metastatic NSCLC, and wherein a tumor sample from the patient comprises (i) tumor-infiltrating immune cells that express PD-L1 and cover 10% or more of the tumor area and/or (ii) tumor cells that express PD-L1 and cover 50% or more of the total tumor cells in the sample, as assayed by immunohistochemistry (IHC).

56. The method of claim 53, wherein the lung cancer is a metastatic NSCLC, and wherein the patient has (i) metastatic NSCLC and disease progression during or following platinum-containing chemotherapy, or (ii) has EGFR or ALK genomic tumor aberrations.

57. The method of claim 53, wherein the lung cancer is an NSCLC, and wherein the method further comprises administering to the patient an additional therapeutic agent.

58. The method of claim 57, wherein the additional therapeutic agent is a platinum-containing chemotherapy, a taxane, and/or an anti-VEGF antibody.

59. The method of claim 58, wherein:
(i) the chemotherapy is carboplatin;
(ii) the taxane is paclitaxel or nab-paclitaxel; and/or
(iii) the anti-VEGF antibody is bevacizumab.

60. The method of claim 53, wherein the lung cancer is an extensive-stage SCLC (ES-SCLC).

61. The method of claim 53, wherein the lung cancer is an ES-SCLC, and wherein the method further comprises administering to the patient a platinum-containing chemotherapy and a topoisomerase II inhibitor.

62. The method of claim 61, wherein:
(i) the platinum-containing chemotherapy is carboplatin; and
(ii) the topoisomerase II inhibitor is etoposide.

63. The method of claim 52, wherein the cancer is a hepatocellular cancer, and wherein the method further comprises administering to the patient an anti-VEGF antibody.

64. The method of claim 63, wherein the anti-VEGF antibody is bevacizumab.

65. The method of claim 52, wherein the cancer is a melanoma, and wherein the method further comprises administering to the patient a RAF inhibitor and a MAPK extracellular regulated kinase I inhibitor.

66. The method of claim 65, wherein:
(i) the RAF inhibitor is vemurafenib; and
(ii) the MAPK extracellular regulated kinase I inhibitor is CI-1040.

67. The method of claim 48, wherein the patient is an adult patient.


SEQUENCE LISTING (22 sequences)

SEQ ID NO: 1 - protein, 10 aa, synthetic construct
GFTFSDSWIH

SEQ ID NO: 2 - protein, 18 aa, synthetic construct
AWISPYGGSTYYADSVKG

SEQ ID NO: 3 - protein, 9 aa, synthetic construct
RHWPGGFDY

SEQ ID NO: 4 - protein, 11 aa, synthetic construct
RASQDVSTAVA

SEQ ID NO: 5 - protein, 7 aa, synthetic construct
SASFLYS

SEQ ID NO: 6 - protein, 9 aa, synthetic construct
QQYLYHPAT

SEQ ID NO: 7 - protein, 118 aa, synthetic construct [sequence omitted]

SEQ ID NO: 8 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 9 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 108 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 12 - protein, 123 aa, synthetic construct [sequence omitted]

SEQ ID NO: 13 - protein, 10 aa, synthetic construct
GYTFTNYGMN

SEQ ID NO: 14 - protein, 17 aa, synthetic construct
WINTYTGEPTYAADFKR

SEQ ID NO: 15 - protein, 449 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 17 - protein, 450 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 215 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 14 aa, synthetic construct
YPHYYGSSHWYFDV

SEQ ID NO: 20 - protein, 11 aa, synthetic construct
SASQDISNYLN

SEQ ID NO: 21 - protein, 7 aa, synthetic construct
FTSSLHS

SEQ ID NO: 22 - protein, 9 aa, synthetic construct
QQYSTVPWT